SEQUENCE LISTING (184 sequences)

SEQ ID NO: 1 - RNA, 23 nt, unidentified
  source 1..23: mol_type = other RNA
actcggcgtggcgtcggtcgtgg

SEQ ID NO: 2 - RNA, 22 nt, unidentified
  source 1..22: mol_type = other RNA
tttgttcgttcggctcgcgtga

SEQ ID NO: 3 - RNA, 21 nt, unidentified
  source 1..21: mol_type = other RNA
ttcacagtggctaagttctgc

SEQ ID NO: 4 - RNA, 21 nt, unidentified
  source 1..21: mol_type = other RNA
tcagtgcatgacagaacttgg

SEQ ID NO: 5 - RNA, 20 nt, unidentified
  source 1..20: mol_type = other RNA
tggctcagttcagcaggaac

SEQ ID NO: 6 - RNA, 22 nt, unidentified
  source 1..22: mol_type = other RNA
actggacttggagtcagaaggc

SEQ ID NO: 7 - RNA, 21 nt, unidentified
  source 1..21: mol_type = other RNA
ttaagacttgcagtgatgttt

SEQ ID NO: 8 - RNA, 22 nt, unidentified
  source 1..22: mol_type = other RNA
tgaggtagtaggttgtatggtt

SEQ ID NO: 9 - RNA, 22 nt, unidentified
  source 1..22: mol_type = other RNA
ataagacgaacaaaaggtttgt

SEQ ID NO: 10 - RNA, 21 nt, unidentified
  source 1..21: mol_type = other RNA
ttatggtttgcctgggactga

SEQ ID NO: 11 - RNA, 22 nt, unidentified
  source 1..22: mol_type = other RNA
tggaatgtaaagaagtatgtat

SEQ ID NO: 12 - RNA, 22 nt, unidentified
  source 1..22: mol_type = other RNA
tccctgagaccctaacttgtga

SEQ ID NO: 13 - RNA, 21 nt, unidentified
  source 1..21: mol_type = other RNA
ctttcagtcggatgtttgcag

SEQ ID NO: 14 - RNA, 21 nt, unidentified
  source 1..21: mol_type = other RNA
attcctggaaatactgttctt

SEQ ID NO: 15 - RNA, 22 nt, unidentified
  source 1..22: mol_type = other RNA
aaaagctgggttgagagggcga

SEQ ID NO: 16 - RNA, 22 nt, unidentified
  source 1..22: mol_type = other RNA
ttggtccccttcaaccagctgt

SEQ ID NO: 17 - RNA, 21 nt, unidentified
  source 1..21: mol_type = other RNA
cattattacttttggtacgcg

SEQ ID NO: 18 - RNA, 23 nt, unidentified
  source 1..23: mol_type = other RNA
tgaggggcagagagcgagacttt

SEQ ID NO: 19 - RNA, 20 nt, unidentified
  source 1..20: mol_type = other RNA
actggacttggagtcagaag

SEQ ID NO: 20 - RNA, 21 nt, unidentified
  source 1..21: mol_type = other RNA
tgcggggctagggctaacagc

SEQ ID NO: 21 - RNA, 21 nt, unidentified
  source 1..21: mol_type = other RNA
tgagtgtgtgtgtgtgagtgt

SEQ ID NO: 22 - RNA, 22 nt, unidentified
  source 1..22: mol_type = other RNA
gtagaggagatggcgcagggga

SEQ ID NO: 23 - RNA, 20 nt, unidentified
  source 1..20: mol_type = other RNA
cggcggggacggcgattggt

SEQ ID NO: 24 - RNA, 23 nt, unidentified
  source 1..23: mol_type = other RNA
caacctggaggactccatgctgt

SEQ ID NO: 25 - RNA, 21 nt, unidentified
  source 1..21: mol_type = other RNA
tgagtgtgtgtgtgtgagtgt

SEQ ID NO: 26 - RNA, 22 nt, unidentified
  source 1..22: mol_type = other RNA
gtagaggagatggcgcagggga

SEQ ID NO: 27 - RNA, 23 nt, unidentified
  source 1..23: mol_type = other RNA
cacccggctgtgtgcacatgtgc

SEQ ID NO: 28 - RNA, 22 nt, unidentified
  source 1..22: mol_type = other RNA
ttttttgctggaacatttctgg

SEQ ID NO: 29 - RNA, 22 nt, unidentified
  source 1..22: mol_type = other RNA
tttgttcgttcggctcgcgtga

SEQ ID NO: 30 - RNA, 23 nt, unidentified
  source 1..23: mol_type = other RNA
tctcacacagaaatcgcacccgt

SEQ ID NO: 31 - RNA, 22 nt, unidentified
  source 1..22: mol_type = other RNA
tctcccaacccttgtaccagtg

SEQ ID NO: 32 - RNA, 21 nt, unidentified
  source 1..21: mol_type = other RNA
tgtaacagcaactccatgtgg

SEQ ID NO: 33 - RNA, 22 nt, unidentified
  source 1..22: mol_type = other RNA
tacccattgcatatcggagttg

SEQ ID NO: 34 - RNA, 21 nt, unidentified
  source 1..21: mol_type = other RNA
aacattcaacgctgtcggtga

SEQ ID NO: 35 - RNA, 24 nt, unidentified
  source 1..24: mol_type = other RNA
tgtaaacatcctacactctcagct

SEQ ID NO: 36 - RNA, 20 nt, unidentified
  source 1..20: mol_type = other RNA
atgacctatgaattgacaga

SEQ ID NO: 37 - RNA, 22 nt, unidentified
  source 1..22: mol_type = other RNA
taatactgcctggtaatgatga

SEQ ID NO: 38 - RNA, 292 nt, unidentified
  source 1..292: mol_type = other RNA
aggttctttttctccgccaatggtgatagatttggcaccaaaaaaagaagagaagaaacatgacagtcattgtttccacagtgcaaagcccagagtcttccctggagaagagtgctgatgacgaggattcatgtctcacaacccagcacacaggaatgatgtcacacagtgttcaaattctatcttcagcatagtgagcccaaacaaaaccaattcctttcatgccctctactggatatttgaagttaaacgtcaataacactgcaaagtaaccaaaataatgatacacata

SEQ ID NO: 39 - RNA, 2356 nt, unidentified
  source 1..2356: mol_type = other RNA
ggaagcacactgcggcgattctggagaggttgtccttttctggcatcagcgttgcggctcgaggcaaagctggtcctgtgcctgctgtccattgccgctaaggcctgctgctgccattcaggcgtgtctctctctctctctctctttcatgccgctctctccaccccatcttgggacggccagctctaagaattcctgctacgacactgaattcaaccaggaggtgatgccacactcaatcagaaactatgctactgctgaagaggtatattgagccctgcctggttaacctccagtgaggttattagagaattgggagaggctcaaatcagcatcctgcacctagttatcaaaccattgtgaagtcttgattcctggatggccagaaaacttgtcattggtcaagagcaacattaagatctgcctgttactcatactgatgcttcctgcccagcaagttcatcagagggcgaggatgaattacggttgccatgaatgtccttgtgaggcagttgggaagccttggctggattgtgaaggatatttgaaaagtattttagagtccaccagatcatccatgtgagtgaccatgcagcaaaagaaggagggtcctctcagtgattaaggattatgaagtattaattcttccactgatatctcaaaaggtgatctggacaaaggaacaggtatctcaggattcctccttggtcacttttcagcagcagcctgctcttcttttgcaatctcttcaggatctctgtagaagtagagatcaggcatgaactcccacgggtgttcacgggaaatggtgccacgcatgcgcagaacttcccgagccagcatccaccacatcaaacccactgagtgagctcccttgttgttgcatgggatggcaatgtccacgtatcacagaggagaatctgtgttacacagagcaatgtagagacggggtttcaccgtgttagccaggatggtctcgatctcctgacctcatgatccacccgccttggcctcccaaagtgctgggattacaggcgtgaaccaccgcgcccggccaggagagtgtattttctacacataagttcaggtcatctccacacagacccagtttgcctccctcttttccgatctgaatgcctttaatttccttctcttgcctaactgtactggctagaacatcccgaactaggttgaatgaggtgctgagagtgggcataattggcttctttacctccgaagaaaatatttcatgttttcttcattgattatgatgttagttgtgggtatttcatttactgcctatatggtataaagatacattccttctatgcctaattggttgacagcttttattatatcaaattctataaaactgttttatttttaaatctattgagatgatcaaatggcggtgtccctcctcctttataagactatttaaaacatccttgcctcagagggacaaatcccatttgaacatggcaagtaatcctcttaatataaagtggaattaggtttgacagaaattaccagtgaagcactatgggcatatatttgataggagatttttattatggatacaatttacttgttattggtctcttcagatattctttctcatgactggatttgcgtacgttgtatgagtacaaattttttcatttattctacgctgctctttattatatgcttccttctgtaaaatttggactgtttgttcttcccattatattaatagttgcttcaggtgtaacataaggttattttttaagtacttttctggtatcacaaatattggtgttttgtgtttcatctttcatttgtcaaaatattttttaacttccatttggatttaatgattcacccattcattattcaaaagcatattgtttaatttctatgtgtttgtgaacattctaatgttcattctactaatagttcctaggttcatgcaatgtgcttaaaaatgtggtataatttctgtattattaaatgtaaaactagtcttgattcgtaacataaggcctacgcaaagatccatcttctgcagagaagaatgtagattgtgccgctgttagacgaagtattctgtatacgtcagtttgctttatttggcataaagtgtagtgtaagccagatgtagtgtaagtcagatccagatgatctgcccattgctgaaacttcagtacttaagtcccctattattattgtatggcaatctatctgtccactcagaagttattatatttgttttatatgattgagcacttctgttttgggtatatatgtatttacaattactataacctcttgctgaattaaccccttttaccattatgtaataaacttatttccc

SEQ ID NO: 40 - RNA, 555 nt, unidentified
  source 1..555: mol_type = other RNA
gatgggcgtgtctccatggcatgggttctgccactgcctcttctctcacctcacaacgcagggctgccacgtgttcccaacagcaagaggagtttggggatccataaagacgtttcccagcatgaagacacagagctgagcttgagatgagaactttggagctggaagtggcctcaggaaaggccctagaaaacaagatgcggatgccccttgtctgaagacaggatcaggatgctcccaggcaggatattcctgggaagaagtgtagggtccccgttttatgtttcagaaccacagcattcccaaagatcagatgggaagaacctggtgtaaactcaaagacagattctggaagacctccttcttctctcctgttatgtgcgtgagataaagtcaggtatatctccaaatgctatccctcccccctccgctgactggtgtctttataagaggaaaatgtcacatgaagacagaaacacacacggaaggtggccacgtggaagatggagggagagactgaattatggtgccacaagcaaaggaacacttggggct

SEQ ID NO: 41 - RNA, 3709 nt, unidentified
  source 1..3709: mol_type = other RNA
tttatttccctgcagctctcatcccctgctcatccaagcctccctccttccagatgaataggaacaggttacagctgacccagtttcactcccagcttcagaagatgaatcacgatatttgagtgcctactaggtgcagtgtcactctggaagctgggaacacagtgggaccggcaagccacccccagtcctcgtgaggttaactagcaagacagctagtttgcaggggctggagagacttcttcatgaaggaatacagggacagtcacaggagacaggagcaccaagggattcggacatgtggctgacacagtcttctggagagccccggatggcatcagggggaaacctggcctgagacagtgccctgatggacaaggacaggaatacggtcatacctcctgcccacttgaccaacgaccaggatgagaagccaacagatgatgaatatgaattaaagttggacaactagcaagtttaaaacttggactgggacctagatcttccgattcctacagctagcctccctttccatcttctctgtggtcatcactaatgcagtttttgaccgtcttgtccagaagctgcaaaacagacaacagaagccaggactggccctgccttagatggctttcagtgcaggaaagtatcaagacagtcttgttcgtagcgtatatctctctccaccttttctgctaagcacctcatgtatgcagctgccaatgtaacacctccatttgcgtgtttaatcaccatctcaagctaatgtaaccaagcagacttcttggccttcatccctgcctcctctagtcttctcatctctctctctctctctctctttttgagacagaatttggctctgttgcccaggctggagtgcagtggcaccatcttggctcactgcaacctctgcctcctgggctcaagccatcctcccaactcagcctcctgagtagctgggtcttcccatctctttaaaaaacaattcaattgttcagaacaaaaccctggagtcatatttaattcctcccttcctcttgcacttctaaaccaatcattcatctctactttgaaaatgtttctttattcatccagtgcaccaagccaccatcacctcgaggcaggaatattcaacggcctcataagtggtctccctatttgtgttatattcaatgcatgtcttttcttactcaaaatcctataaaggcttctcttcaccatccacgactgtttcctgccttcctcactatgatcccatcactgacccctcaagactatgttccagcctactttctgctccttgaccaacacccttactctgattcccacttttgtgacttcacatgagtcccctgtctgcctggacactctgcccccaaccttgaagtgctcacccttccctgtcactcagttgaaagtcaaattccatttcctcatgagggcttccctgaatacccacttttaattaactctctgcctcctcccatcacttctgttttaccccctcttttagttccttcctatcttagtttcataagagccagggccgtttccgacttctttccccgtcaagcctccttagataggacaggtcctctgcctcaccgactttaatcagtatttgctaagtaaacagagcacaggaggatcctgttgtcatcaaagagcagctctgcctccaatggcgaaaggattgttggaacctcatccttccttcaagttctggttcaaaaaattccatgctatgcctcacatggccttgcccaggtgcctgtttgatttgcctgggttgtctgttctttcttccccactgtcctcatttgccatgtggctcggtggtggctggagacctccatgctggcccctgggagcagctgctaaggctcggcgcctcaggacaccttggcacctgtggatatcgaccaccaggcttctcttctgttcatgtctctgcaagaatgagaacctgcccccataccatttgtgctgtgtaggcctcaggaggcatggactcacgcatccagcttcctgcaagccctctggtacatcgcagtgttcacccccagctttctattacagccaccacccagggactctggtgcaggctccttagagagaggccaggaaactgccgaggtgataagtgacagcaggtctcaggggaagagaggacttctctccttagtaaagaacctgtcgaggtcggaaagacactccaggctcaaagccaaatttgaaagttaggctgacagcccaactagggcagattggaggagccaggtggagagaggatgtgggggagtcaggaaagaggtggaaggagaagcctggaagagtggctccgctcctggagtatggccagcggcacttcgtcaggacctcgaccttcacccactagtgggagggtgttcctcgcatgacttgtaggatcacctgggaaacacacaaatggaagaatttaaaaatatctgctaggacaccccgtgtcccccacaactcatcacagagcaaacccatcacagagcaaactccttggctacttcttgtcttgggccgtcttttcatctggcctgtttgcctgactggactttggggtctttgttcccagcatcaaaatttcctaccagcgactttccatcacagctcccttccagtcttctggaaattgtcagatgtgcctggatttgcagggcctgcacaagatatgctgtcgatctgggatttttggggatttttgcagttcacactccaatgtgcaacagcaaccaaacggagatggaggacgtgaacatgaactactggagatgctgatgaaacgtgtgtaaggaccgcaatggtatctgtgtcctgctcgtgaaattgaccagatacagcggcttccttttgcttccgaaggcccggattgacctgacaattgatatgctttgctctatactccaactcccattgaccatggggtgaaagcaccctttctcggtcagcctcactgcttccccgcagatggagggattttctgcaggagcatgggccctgggccttcctttcccactgtctcttgaaagcctcctccagatgtgctgcctggctcagctctttgggtgacttttgttctgtctcatgtgacccatcaagattcacacagggctgtgtcccgggcagggctggagtcgacattcagggaggagatcactgttctggaacagagcagtgattggtttaattgaaatctgatagatcatcatgtaaaaagctggcatgtaagcatgctcccggagcctgcagggccctgcgcggcctcttgcctggccatcacgtagaggcggtcctcttgcatggttgggggcctcacctgcaccacgggcccccaagccaaacagtgagattgtacggagaccatcctggtcaggtcttgcagtttgcaccaggtagccccaggctatcctgccgtgcaagagctgccttctcagcacagtggagttctacggctgcctcggctgcagaggtcagctctggagccttgctgagcacctccatggatgcattttgcaaagggccgagtaagaggcattaacgtggcctgtgcttagttcctgacagcaagcgggattaaacccggattaaaggatcagccctaaaccaaagcagaccagtaataggtctcatgataaaagcagccaaataataatagtaaaaatctattcttggactt

SEQ ID NO: 42 - RNA, 558 nt, unidentified
  source 1..558: mol_type = other RNA
aaacaggctgaaatcttttcttcttgcattgctatcttcagcatcttatatcattcagtcagctaaattccatctttgtgataaatcccccacaaatattttgtgatctctctgatctaagcagtgttaaaccttttgattccttgtatccttcctctcccctttcttaaaatagtctgtagcgctcatgtcaaggacctgctcagcttccctttctctaaaattgcataatcctacaagaatattttcccttcttttccttcaggagcaaacgttctcaaaacattcttaaatgcaaacccgcaggcacagcatgtgtctgtcaactccaagagcatttggatgttcgaagggagaggatgtttgacatctaacccgtcgtggaaagaggagcctctacagtgacatgaaggcaatggattctcagcattacaaggcaagacagaacttgaaggaagaaggctgggaggaaaccaatgttgaatgaacactgactctgtgctcagccctgagcaagcagtggaatgcccctgggctgtgagccacgaattcaaatcc

SEQ ID NO: 43 - RNA, 703 nt, unidentified
  source 1..703: mol_type = other RNA
gccaacaggagctggtaatcaggatggcagtgaggcaggcacttgcttgcagaagaacaggcctagttttttaagaagccacaaaagtgtctttggatgattaggatctaaggggccaacagctttgcaataacagcataaatattgtgggctggatggacaggcaccaaatggggacattccatcctgctgtgtgatgagaaagctgccacttttgaggccttccaaaacaaatcaacactccatatgccagaaggaaaagcccaggaaagacctattccaactggagatggtgaatgtcatttcttcaagaagctgagatccagctggtactgccaggtggagagtgagttgggaatacgaagggagaatgaaacaaaactgctccgcttagggaacttggaaaggaaattgaaaaggcaaggcacaggctggaagaaaatatttgaaaaagatttatgtgaacttctttctggattatacaaaatatttttagatattcttacaactctaaaataagataactccttaaaaatctaaattctaccaaaggagagataaagatgacgaatacattcaaagatgctgaaaatcacttatttattattaggaagttacaagctaaagcataatgagataccactacaacctactaaactgactaaaattaatcagactaaccaggtgttggcaagaaacaggg

SEQ ID NO: 44 - RNA, 1448 nt, unidentified
  source 1..1448: mol_type = other RNA
aggcacatgcgcagaatcatcgtggtgcacggctctccctttgcttcttcggttgcagtcctcttgcttcttgcgcgtgcgtgtagcgcttttgcaaagccgcggaggtgaagtgaacttagaggttgtggggccgaggggtcgtcttatagctaccagcccacaggcatttagtctacgttggaggtaaacaaatacgggtcctgcttaggagaaaagaaaaacgtcttacagccagtgtctaaactccaaacaacggaatgtatcaatgagaccttgtatatggatacacgtgcatttaaaaccgccctgccggcttgtagagcttttgccgttctccagcgctttacaggggttatcgcacttaagcctcggaacaactttaccagatgaggacacctgaggttcagattaagaaatctgccccaaagtcttagaactggtattctccactggttgaatgcaaatggaagccagaggaaaagggaacccttgacacagttttcatatagattggcctccagggcagacagcagttgctctggctatatcaaataaaagtgtcaagagtgagcatccttgccttgtgctgaatcacaaaggaatacctttcagtttttctccattgattatgatagcagtgggcttttcacagtgggctttactgtgttgaggtaccctaatgacaagttcaccccagtgttttctgaggagtctacctggaaatgagaaccatctgtccttcctatagtagttcttctcagaatgccgactctatgctaatggaggaggtctgatggccgggaggacaggaagaaggaacccacaatgaagcaaaagttgcgtttaactctgtcctcaccattcctctcctggacctgtcctaaactggaagctgggggaggatggagtgctggaatgctgcagaagtctcatgggagggcgcctggtgttcaccacatgtgtacactgttgcacgaccagatggaaatttgaatgctggggctcagtggaacacctgaaggaagctggtgaggcagatgtcccgctatgccttccaagtgtcatgcaaccagttcatgcctgtcagcatctctgccggtggactctgtgagtcagcaaatctacagagcagagagccacaagtggggacagatgtggtcaaatgccatttaaatcagtgattctcaacccaggagttgtcatggcactggccttgagtgagctggaccaagggaagtggtagaattccccatgaaggaccaagtgagcagatagaagtttctccattttaactttttctgagtcaatttaaaaaacacctagtgcctttttctgtacatagtgcctgtgggtttgaatttaagcctacccaaatgtgcttttatcccacctattttgaaccttgcatgtctccgggataagaattaacataagctataaatatt

SEQ ID NO: 45 - RNA, 602 nt, unidentified
  source 1..602: mol_type = other RNA
aaaggctgtttcatcccacatgggcacgaaacataccacattttacggaggatgtagccccagagaagagggaggtggagaagcataaatgaaaggcaccttttccttcctctagaaggtaaataattataaaatatgcacagagaatttctgaccttgtgacgtttgggagtgaggagatcccatacagaggcatccaggtatttccagagatcctgtggcaggtgaggtctgccctcgctggagccaactcgtctataatatcttcctaacagcagataccatagaggagaggtcgaggtccttccactgaagcaccaagtggggtatataaagccacactgcccatgagggctgtggtgtgagtctgctagccgtgagagacagatgcattacaaagggatggatttagtgcactctttcctctcttactcacagcctgaacagccagtgaacaatgaaacaaaacttcttcccaagcctcaatgaggattgtgccatagtttgaaggtaggagaatttcaagccattaatcgtctatgaatactgaataccagtgagaatggctccttcaggtctatcagcttattgctgcttctgtg

SEQ ID NO: 46 - RNA, 563 nt, unidentified
  source 1..563: mol_type = other RNA
ttcaaagaaaggaacgatgtggacctgacctcaaagaaatccattggagaatatgacagatttagatttaatgatcaactttacttttcctatacagatgggaaaacagcctgcaaaaatgttaagtagcttcctcaggatcaagtactattggagccagcattggtggtcaatcctgtatagaaatgacttcagttgtagatctgtgaccttccttacttaccttcctctaccaaagtgggtcaaccaaaaccgcatggcgtactactctctgaagcctctactaccctgctcctccgtgttgacatgtggtcaggcaagccaggacttactcacatcagctacatcagttactgggatggagaaaattgaagcctagaaagatcaagaaactttctccaggccataaatagaggaatcaggattcaaatcagatagaccccagggcttgttctcttcaacaccacattaccctacattattattcaattattaaataaaaccttgcattagtggcatttccaaatgcataacaaaaaaataaaaaaaagtaacactggtca

SEQ ID NO: 47 - RNA, 670 nt, unidentified
  source 1..670: mol_type = other RNA
ctgcgagaatcgaggcactcgctggcgtacccatgtatcgaaatgagttcacggcctggtaccggcggatgtcggtggtctacgggatcggcacctggtctgtgttgggctcactgctttactatagccggacaatggcgaagtcgtcaggtctcaccctgtcgcccaggcttgagtgcagtggcatgatctcggctcaccacaacctgcgtctccgggctcaagtgattctcctgcctcagcctcccgagtagctgggattgcaggcgcaggccactgctgcccggcgaattgttttattttttttttgagacagagtctagctctgtcgcccaggctggagtgcactggcgctatcttggctcactgtaacctccatctcccaactcaagcagtcctcctgcctcatcctccccagtagctggggttataggcccatgccaccacgcccagctaatttttgtatttttagtagagatggggtttcactacgttggcgaggctggtctcaaacacctgacctcaggtggtccacctgcctcggcccccacaaagtgagattgggattacaggcgtgagccaccgcacccagcttttcttttcccttccttttcattccttccttcattattggggttggagagagtggatgtaattcttaccttagtta

SEQ ID NO: 48 - RNA, 1330 nt, unidentified
  source 1..1330: mol_type = other RNA
aagtccttctggctgcagttaaaagggctcaccagcctctccctgaagtcttggcccgggagcctgtgtcctccaggtagctgtgctgtggggagctggagaggactgaagagctgaggaggcgaaatgcagaaatgataatgcctcacaagctggttgtacagattgaataacaatgtgaacagagcaccgagtataatccctgaaacagcaaaccagaggtcccgtctggctggcatgaatggaagggcaaacgctccgaccaaattgctctgggaatgtcattctaattattggatcctgacccagcagcatgctgataagctctgggttggagacgccctatctcagaggagctccaaacctgctcaaggccttttcttgccctctgcaactttcctgccaagcaattagcctccagggaagaagaggaaattggaacacggacacgcacagaggaaagaccaagtgaagatacagtgagaagatggccaacgacaagccgaggaacagggtctcagaggaccccaaccctgctgacaccttgatctcgggctttcagcttccagcactgtggcaaaattcatttctactgtttaggcctcccggtctgtgacatgttgtcacggtggccactgaagactcagacagcttccttctagctctggttcttctacgggtgtgctttcaggtgctccggagggcccggtggcagggaaagccaggtctccaggctcctggcccagggcgtgtttaggaggcagggccttctctgacacaagtatgaggagcagagaggtttctcttcttcagtctccactaggtcctggcctccatggcttctataggcagggacctggccagggcattcaccagagactttgacagggagtccaggcccagaccccataggcagagccaggagccctgtgccagcttcccagggagcaccggggcccaacgtagagtcaagagctggcatcagcacaggtgcaaggtggggttgggaagggcatggcagcttgggggtctgcagtgcacaggtggcctggcatgaggtaggtgggccggcttgaaggtctagaagatggatagacaccagagacttgtagtctatgtctccttcttgctgtcccctggatgtcacctcccctctgcccccagtgctgaactaacccccttcatcttttacttgggactccatcagtgactcatcattgcttttggtgtgacttaccagcggccgctccgtctgcctcttccttcccctccagggtcattttgaacactcttccttcctgacaactaaacttttcactgtttcccatca

SEQ ID NO: 49 - RNA, 2741 nt, unidentified
  source 1..2741: mol_type = other RNA
atcgctcgctgtcgtcagtgctaggaacctgcgcccggctgagctgagcgaggcgagaggagaaagcgaggcccggggacgggactgagagctctgaaaggagggaggcggctcccgagtactccgcgcggggcaggccccggcctcgcctcttcgcccatttttaaggctcgagttagaggccacctcctccgagaagccttcggtgacaccacctctcccagacggttcccctttctgattcccacagcattttccaaccattcacggtcagaaccactcaccaggacgtgttgtcaccattttaccgctttgcctcgtctcttaactagacgaaatgggttccttttggctctttggaaagggaggctgtcttctatttatttccgtggtaatcccacctcccacaacaagttccattctacagcaatgtcttttctcttgctgagaaccattcaagtctaagttttctgccgtcttccttcctcattgtggtttggaccttctcgagaaagaatctggcctcagtctccctcttccacactcagggaccgccaccatcacaggcactcggtagatgtctgctgaatgaggataacttctacccaacaccgtgaagcctggcccagggtctggcatacagatgccgtgtttgtaagtcctggttgctcatctcgcccacttgcacagcccacctgtgtggccgactcaggattgacccacctgctctcttcaggccatattcccacgcagaagtgggtcccactcctcaggcctctgtgctggccccgtctgtggggttagcaagcagccctgctctccatttccagctcagaagcaactggatcgttacctcattggtgacaacttgactgtgctgcccagagctgataagaaacatcacccggggaagaccagctgcctcttccttgcttcctaatcagggtggaaatactgctaaatatatcacaactctttgcaagcatcgttgggtaatgatcgggacatctttgtgggttccctatcattttctcacaaagttgagcctttcaaggtcagttctaggccagccagtctgggagtcattctttagaatcagaagagaatttaagaagaggagaagataagattgtctctaaaacctgctaatgagaactacaggaaggtggggatgctagctacttttaatatttctggtttgaaccccttcctccaccccagaatgaactgagagtaggtacagaaatagataatgcaaagacggttagggttgagggtaagcagaaaactacccatctagaccgaaggaaggcttgctaacatatcagtaagcaaacagctttctaatctgcaggtaacaagttttacatccctgaggacactttttgagataggactttgtttgtttgtttgtttgttgttttttgtttttttgagacagagtctcgctctgtctcccaggctggagtgcagtggcatggtctcggctcactgcaaccttcacctcccaggatcaaacgattctcctgcctcagcctcctgaatagctgggattacaggtgcatgccaccatgtccagctatttttgtgtgtgtgtgtgtgtgtgtgtgtgtgtgtgtatttttagtagagatggggtttcatcatgttggtcaggctggtctgaaactcctgacctcgtgatctgccctcctcagcctcccaaagtgctgggattataggcgtgagccaccgggcccagcctggggctttttgagaccctattgaaggctgaggaagaatctggaagctaaagagtaacccattccaccacctgctctgaagatagaagaagcaaaaaaagattaaattgtacaaaaagcacttaaattagacaacagaagaatttcctgatacaaagtgggtgtctaagatgtgttaaacaagatgcctgctccagccaggactcagacactcctggatggagagcatcttcacagtttgggataaaagtgactatacagagtggtggagtgaaggctgcaggcccctccccaaggtggcatctaaccccggaggtatcaggacctcagaggctacccacatgatttccagctgatgagcagaaatagctgtggcaggtgcaggcttcacttgctgccaccaacatgggagctcaccaaacacccagtggccaaaagaaccagggtctctctaagccctgggctcaccgccaggcaagcttgggtgcttgggagggccgtcgttcttcacagtgcagcccctggatttcttctcaatttactcaactctctctcttgaatcccatctgtgcagcttagaagtcacaaattagaaccccccaaggcaaagctgaccatagatacaaatggttttgtctgtatagtgtttaagcacttaaaaaattcattgtgaacatttaataattagaaagtgtcaaaatgaaaatatctatagtttctctctaaaaaccagaagatctgttaacactgatcccacacttctatttggcaatgactggcccctaagacagggggtatgtcctctagctcaccacagtctccacctttacctactgcatccctgctgagacaaagtggtacatgccatttattaaggatgaatgctgttttttttaattatacttaatcctcatttgcattacctgccttcattctggaggtatttgagtttgcatctcctgataaagaataaatctcaaaattgtcatctctaacttc

SEQ ID NO: 50 - RNA, 551 nt, unidentified
  source 1..551: mol_type = other RNA
agcccctggtatatccaggacatcagcacatagaaccagtgaaagggtcctccctcatagtgacgcatgtcatagactcagatttatacaaacaaatgctgagcaaaccatgaagagtctctggtattttcaacaatatcatcacagaggaactgtattgatttccttccttcagtggtccaagaacctgctgctcccgggtcctctcatttgaagacacccaccgcctaaggatgaagagaagctccttcttgcagatctgaggctgatttcccaactcccaggaacaccctacccacagcctgttccccagacccaggaattcttgaccaagactacaaatgtagcccatttgacatcgaaaacattgggacaggtgtgagagtgaggaaaatggggagaaaaaagaactttgaagacaaggatttgaagttagcatgaagcatggaaaggaggaagggcaagctttctgcagtgaggacagtgcctgccagagaaaaccagatcctggggaggggtttctccagaaagagaaagtcaaagctgcaggt

SEQ ID NO: 51 - RNA, 3202 nt, unidentified
  source 1..3202: mol_type = other RNA
ctcaccaatgtgggtgggcctcattcaatccattgcagacttgaatagaactaaaaggaagaggaagggcaaatttgttggctgcttgagctgggatattcatctttctcctgatcttggacatcagaactcctgattctcaagcctttgggtttggactggaggcaccagctttcctgggcctccagcttgcagatggcatatcatggaacttctcagcctccaaattcataactctaatacaccagtacaatggaaagattcctaaattcaaaagccagaaggctgggttcctgttcccaccctgccttttaccttctgtgtgttcctgatgaagacacttcatgctccactatttacttacctctgaaacgaagggctgacccagatcagttgttctctgacctgcttggagggactcagaggctgtggagcaacagatttgggaatgaggaatcgttccctggaagagtcagagcgctcgtagacacattctgctggattgcacgagctcctcccctgggaaatccactaagactagaggaaagaattgcttggaggattcagaggctgaagagtggacagacggctttaattgagaaaaagaagcagaaaattgaggaagatgtgaggcatcaatgccagcagacgacatgtgacaggtgctagccacatggatcacactaaatttctgtcatttatatagataaaaaagaatttccaaagagctcctccataattcagcattaaccagctttaaatcatcacctttctttaccttttcaaagcacttttatctagctagctttcactttcagagtggccaattcaggtgaggattctcaatcctgttctgcagctgaggaactcatgctcaaaagggctaagagtttattctcttacatccaaacaccttgcttttccaaatcttgtgctcactttacctacacattttgatttttccaggctgatgacaagttagacatgcagactcctcaggctcattcataatctgtatagacattacaggtcatgcaaatttaagaaaaggctaaaaggtgcccgtttgcacccaacatgttggataatgccccggtaaagtggaaaatcagtttccacagcctcgctctgtaggtttccttggaattgtaaaagcataggactctgcctcatatttatttctatacggcaatgaggatataaaggagcagaccgaaaagagaaaggaattttaaaagccatggaaagctgccctaaatgtggttggatatagagaggagagtgctgaaggcaaacagctgtgccctctttgcctttttgctgaggaaggggatttaagccaaaagaacacacgtaaacgtggatttcttaaagttctcactgtaattatgtagccttgggagatgaggaattgtaaagtagatgctggcttaaacaaatgataaatccttgaggtgatgggcaccccattttccctgatgtgattattacacatagcattcctgtatcaaaatatcccatgtaacccataaatatatatacctaccagtatatttctgtggctaatctgaaagccacctgcacccttgtgaagacacccacagagacttctgcctcccctcctaccctctggcctcaagagctccctgggctctcctgttgcaggagggagagggagagcttctttaggttcctggtaaggatctctgaagacaattccttaggcttggataattcccagacatttgctaaaaagcagcatcctccaaagctttaatttggatcacaggaaaatgatgctcccgcctcagattcaaacacagttttatctcacactgcaagctccctggcatgggcaaagtcctcgatgcctttcctggtggtttccaacatcaaggacatacttgttcctcctcctagctctctccctcagccaagtccttccttacaatgaattgtcctgcagaactccctcttaagcttggcttctgggacagaaaacctactttctgaaagggtatggggactgtcagagcatgaactggactcaagctcccaattacagagcagacacaaggctctgcctgcaccaggccatccctggatttctgcagctccaggactctcttccagcatctagatgcagctactctcattccagctttgtgtcctgtttgatgcaaaatctttattccttcctgtttcaatccctgttgcatattctgtgtgacaaagcaagaagctggaaatcgagttctgagctttatacccagctctgccacaaatgtgccatgaccttgaataagtcacatgacttatctggacctggattctctcatctgtaaaagaggaagctgagctatactacttttaaaatgtctccagctttaattttattagaatatcagctgcttcctcctctggtttctcatggcaggttgtgcattcttctctggagacaccaaatttcattggagtatctctccttacaaatgtatttccccactagattggcccctcaagaacaaggattgtattttagttacctctgttatctctgtacctggggaggagtgagaaaaggagagcaggaaaggcaatggtatagtaccaacatttgttgagcttctattatgaggaattgtctatgctacacattgtacataaagtatgtatgattcacagtataaacattataatctggaatcaacctaaatgttcattagcagatgaatggataaagaaaatgtggtatatagacgcaacacaatactatacagccataaaaaaaaaaaagaatgaaatcttgtcattcatggcagcatggatgagcctggaggacattacgttaagtgaaaaaagtcaggaacggaaacacaaatgccacatgtcctctttcatatatgggaaaatttaaaaagttactctcatagaagtagagagtaaaatagaggctactagaggctgggaagggtagaggggcaagggtcatagggaaaagagagttaacggatacaaattacagctagaaagatgggaagagtgaattccagtgttctaaagcagggtaggtgactacagttaatgattatttattgtctacttattgtatattattgtatattttcaaatattgtatattttcaaagaggattctgaatgttcccaacacaacaaaataataaatatttgaggtgatgga

SEQ ID NO: 52 - RNA, 614 nt, unidentified
  source 1..614: mol_type = other RNA
gagacttgagatattgcagatcgggttgaggaaggtcaaaataattagtcattggagatgatatgaggaaaggaaggagggggaggttgggggagtgggtgcaacagaaacagttctagtgttggagccgaggaagccaactctgaaacatttggacctttgttgtctgacattgcttttgccactccttcttccttcctcccacctttgccttccccaccacctcctccaaactcccctgccttctatctctggccccactgatgacccctccatgtgagtcgtaggcttttactacccatgatttcttttctccaagcaaaagagagaagagtttatttcattctcagcagcatccctggccagcctcgtcaaaggctgtcaaataaacacattgctaggaggatcacctcaacctgtaggaacttccttcctgccaagaagctggttcattcatcaccaagcatcatcatcatggaattctcaccctgctggaggttcgagccaagacatagaaagaggacattacctggggcaggaatcaattgcctatcactggggatgcctgcagatcattgtccagagtggctgctgttcactgagtgtttcctctg

SEQ ID NO: 53 - RNA, 250 nt, unidentified
  source 1..250: mol_type = other RNA
cctgtcatcgtcggtgaagagggacaccaccagataggggccccgcaccagcgcacccctcggcgcttcctccaccggagtcacaaactcatactcctgggcgctcgggccatagctgcccaccatgtagacggtcttgtcccgcgcaggccccggcggtaggtgtggtgcagacacttgaggccgctgacaatctccctgtggacttgaaggagatcttcactctgtaatcaacaccttccttcaggac

SEQ ID NO: 54 - RNA, 1660 nt, unidentified
  source 1..1660: mol_type = other RNA
gagagggcggcctcaggaacagcatgagtgggtgcagtccccactcagagcctgcatgtcccagcagcccagagaccatttaagatctctcctgtgccggtgcgaagggtaatcttctggaaccagagaggggctggcaggtggtcagctgcccttagagctagaggtgagaaattcccatggtggaaaaagggccagaaacatccaccaggcaagctgcccagacacaggattgggggaatgtcccctccctgggtaaaagcaagtcttttctggtctttaatgcacgacgtaatacttttacagtggtgggtgaggtgaggacagtaggagtttggaagagggaggtaatattagtgcatgtttgttaaaaatccctcccaggtgaatgacttttttcttcatctccaaaatacaagagaattatttttaggtctccaacttaaaaataatatctggagttagcaggtaggtggatcagggccagccctggtcccttagccccccacccctgtcttagcctagggactgttgcccagggacccgtgggcacagaagcaccccccaccccacgccctcgggacaactgccatttccacattatccccatctttagcctgagcgtggattaagacagaggagatcacagacacacaaggttaaagatgtttcttccatttcagatttccattgtaaacagaagtcctgttaatccctgcatgcctttaataaaaaaaaggaattagttgataaaaattggtgagtcacattcatacattagatttttttctttaactttcaggatgttggtatattcgtaagaaaaatagcctctcaggatctagccaaaaaggctgaaagtcaatcagtttagtaattcaccaaaaacaggagctaaagaaaggaaaaggcaactggaggctaggattttttgttgctgttgttttttttttaaataaacatcatcatctatgtgtaatcaaattcccatattttcttcctataaagaatttgctttagtttttcaataaggcatttttttgtcatccaaacatctcttccttttaaaattttcttagagttaaaaccataaataagaggatttaaaccactaaaatgacacgtgccaacatcttcattcagccagacctggtaaattctatcaaaactagacagttaaataagaaccacgttataaaaatattagccaaaaaaagactattagataattctgcaaactcaaatatgaaactgtactaaacaaaatatgtgcaaaggtacacaagcatagagccacgttgggggttatgctcagattagttttaaagctcgctctagtggatttaattcaagagttgtccacggtggtggtgtttactttgaactcacacgagtcaaagaaaaataaaatatgcacaaccacttccccaaaaggtgtcttatggagccggtgcttcatcgtgtccacacccaggactgcctggggtgtccctagggctggggggtggtgagagcaggccaggctgaggggcctgccttggaaggcgagggtctgagggttggggctgtaaagacagccctttcatcaggaagctggagctgaaaagccctaatacccctaatccccaaactcttggatcccgtcccacagacgagagtc

SEQ ID NO: 55 - RNA, 1531 nt, unidentified
  source 1..1531: mol_type = other RNA
acccaacatgctactcagcccaattcccaaccctttgcaccaggtttggcttctgtttttatttttgcaaagtctgttcctcttggctccaacttccgtctttggagttgttaatggagaagttagggtgctatgtagattcaccaggcgagttcagtactactaccctggggatgaatgttggtgagaagccattccagatagacccccaacaactctgcttcgttgaccctcagaacagtaccctgtgggactcctgagccgtaaggagtctccctggtagcgcgcagcggggccaggctctgtgctccggcccccgtgcggtgcccagctctgcaggttcccgaaagtgcgccccggggtggggttgcaatccgccccgtgacggcataataaataattagagggtttaagcgtgttagccacagctcatgctaatgagccagataagggctcacgctaacccgaagtgacagtttgcatataggagggaagaagaaaaaatcctgcttccccccccacgcacaaacttgattgctccttctcaaattcatgtccccaaatcccttcccccattcctcggacttccctcttctcacatgcccccacttcagccacaacacacacactatccgtttttgacccttcctttgcctttcaccgggtcagacggcattgtgacgtcacaaaacccctgggtgcctatatctgacctgcgcggaaagttcattctgagtgcgtcctggcgagtctcagcccccaagcgggtggaacgcaccaagggtctcatccgcgaggaagggctttaacgctcagggtcaggaacctctgagagctcttggggacttgcgcgcccttggatgctcttgcgttcaacagggctaggagactaccggctggggcgaggaatgcaggcgcctagagccttggagctcctgaaacgttcctgccgcctccggaaagcaagggcagtttaccgaagtgactgaggccaagtatttgcagcacgggcagggaccagtaactgattttcaggccttttgagaggaagaggaagagagaggaagagaaggatttctaatagtagatattcactctcatggactctttaggaaacagcaacaaagacaagattaagaacaccaaattgtgtgactatccacacaaaaaagcatcttcataagaaccaaaaatcagagggcaaggaaaaaggctgaatgcaaaaccagaatgtcaacagggtgctaacggagaccaatggaaggggcaactgactctccccagggaaggtaggcaaggagattgagggaagtagcaatgaaaggagagcttgcaaaatgaagtcatcagagctttgcataagaaacaattcatggaccttagatcaagtagtgaggattaaaactatgtctatgtgaggctttggctagtttttctacctaagacatccttacctctcctttccacttgggaatcatcttcctttcctaaccatcatcatcaaccctcaaggcagtcaggggcatctgtg

SEQ ID NO: 56 - RNA, 823 nt, unidentified
  source 1..823: mol_type = other RNA
gcgacccccgggcgaccccggcatcgcctggctgcgccgtcccggcgggccggccctctccacgccctctcctggggtttgcggtgggcacagctccctccagactgggtctctccactagcctcctccccaactgcaacgtgggaaccagagctatccgagtcacgagccccaagtgcacacaaagcccccaacggggccacggcgcctccacgcctcgctacctccagctccaggagcgcagaacgcagcgccctgggtcaccgcgtctgccctgcggagcagccccactgaggggaagcctcgggcaggagagttacacctgccatctagcaaaacagacctcacccaaggtcaatgatggctgctaaccttggggttgatgggggaaggtggatgcagcagaccccagggaaaacagagcaccgtgaggaggagccagaggaggccggaggcccttgtctcagacatccagctctgagagtgaaatgttgcttcctaatggactttaaattaccaacatcgctaaactctctcttaatagacttgtaaagatttgcttgctacctttttcctccttcatgtagaatttgtaatcaagagcccaattttgtcatttggaaatactaattatctaatcacggcatgctgtataacgtacttgtctgcccaattagctaggacaattattaaaattcagaggcctaattatgtctcaatttaatgctggcaaacttgcatacagagcagtaattaaaatcctctctttccctctgcctttagggcaggggatgattaggccctgtcagggggtgtactgaac

SEQ ID NO: 57 - RNA, 511 nt, unidentified
  source 1..511: mol_type = other RNA
cttattcacatgttttttaaagtatgagctgctttgtaaaaataagcctggtctcttaagtgaaatcctcagtttgtgcctaatgaatttatgctgctacttttcattagctcttctagtttgtgatttatagaaattgctgcctatcaggcaccatgaggttcctgaaagtttgagttgaactgtgaactgttctgcagcagatccatgtagtatctgatgagtactgaatactattaactcttctcttcactctgtacagggaacaagtgtcctttctccttttctccatattggatcagtgattacattagctgcaatgatctacaagaaatctgcagttcagctttttgaaaagcatccctgtctttatatactgacatttggttttgtgtctgctaaaatcactaataagcttgtggttgcacacatgacgaaaagtgaaatgcatttgcatgacacagcattcataggtccggcacttttgtttctggaccagtattttaacagc

SEQ ID NO: 58 - RNA, 448 nt, unidentified
  source 1..448: mol_type = other RNA
cttggaaagggtttggaaccaagtattctctggactcagggacaccagacaactcttgggcccggactgggggacatctcagaaatgaggtttcagtgaaagcctgcacaccaacaataagaatcccagacccttctccagttggctcccaggatactggtagagaagtttatattttgcaggcagaagatggaaggttttctctccaagggaaatgatgagcccaagaagaaaaagacactggagggaagctgcaacatgaaaaactgtcaaggaatagcagcacactcacaacaaacacccttcgaagaaaacccaggaagacccatgcaatagattcctgggactcaacttagaaggcatggtggtactcttcactatttgtctgcagtgatttgtggaataaatctctcacttttcctaaccctactttcttttgttattaaaa

SEQ ID NO: 59 - RNA, 1268 nt, unidentified
  source 1..1268: mol_type = other RNA
ctctccggtgcccgggagaggggccgggcgggacggtacagcaaccggaggaaccttgattccctgccccgcaagcccagcaccggttttgccgccttgtctcgaagggtcaaccaggccatctcctgcctcgggacggagagcgccctggaaaaggcggaggggccgaccttagtcacacaagagcgatggcaagattttcacccaagccatctgacttggaacccatggattaaccaactgccactggaggcaaatccagagaccaagggagcagtttatacagaaacagatgccggcattaacccacagggagttattgttacagactgaagaatagaaaaagaaaaagccaaatagagccttattccacccagcttagcttccatagaacattgtgaatgagagagtagattcttgagaaaatcctggggttacccagttttcatgactcgtttcgttagaccatcatttcccaaagtgtgttctttataaaaagtagttcttcagaaggctggaaagtattgtttggaagggggttccgtgatgaagtcagtttggaatgcaggctttacccagttttgagaagattctttgcaggacttctcagggcctttgtgaatctccaaagaaagaatgtagtgtgaagtatgaaaactttctcctggctgatggaacacactttgcaaagcacttccacagaccaatatgatttccagtaagagctaaataaattttgttagttgttaaggcataaaacattaatataggatattacagtcctaggcttacattacttaatttggattttttgcaaaaattaactttaatgtgaaaaattaggagacaatatagagaaaattcaagagcctctccagataaattcttagaactaatgagaaagttcactaaagtttctggatataaaatgcccagggcaccggagggggaaatactttcttgtatattaatagcaaacaattagaataatttttaaaataccatttataatagtaccaaaatctatgaagtatctaagaataagtctattaaaatatggccaaaacattcatggagaaaacttatagcctttatttaaagacataaaataattcttaaacaaatggagacattattaaatatgtttatggataagagagactcaatgtagtaaagatgtaatcttctccatctttcacttcctttgcttcctaattaagacttatatcgctcttggctgcgcacagtggctcacacctgtaatctca

SEQ ID NO: 60 - RNA, 2083 nt, unidentified
  source 1..2083: mol_type = other RNA
ttggacacacagacacgcagacacagagacaccggggcccagggccctcctatggaccctgcccgctcccctcccattgtccacggctgtccgcccacccccattctccaagcttcagccccctccttagttcggcatctgcacagcactgaagaacctgggaatcagaccctgagaccctgagcaatcccaggtccagcgccagccctatcatgaccaaggagtatcaagaccttcagcatctggacaatgaggagagtgaccaccatcagctcagaaaaggtgagggccaccttgccctgcctctgcaaggcgagaatttggcggttctccaccccccagccacagctcctactcttgcccgtgagcctggctctctctctgggtctgtctccctcccccaacactgggaaaggtgtcggaactgcctctctcaggagaggggcggagtgtggggttggagtccctttattggtgacaggtgcccaaagctttcctgtgcctcctggccctcggaggtggacccgggggtgtgggaacagctggaagctggagagatgaggtcactgtcggcttcctatgacgaagtcacgccccctcttcctttccccttccaacaccacccagggaccccggtcgtgcgagcgtgtgcgtgtgtgtgtcagtgatcagtttggtgaagggggaaaaggtttctgtgaagggtctgaggattctgtgaggggggcgatgaggggtctctgacctgagggagaacgagactcttttgcttcaaaaacaaattccccttgacccatttctttgtcctccgagcagggaattgtttaggctgagcaaggatgaagttcgtgggggatggggtgcagcgcgctttgacggaaggagggtccgcagcggaggagacccggcagggaggcccccccaaccctccagctctcagggcacagggctaacgtgtctcttccccctgctgggtggaagacttgagggcctgaatggtagctattgcaccttctctccctgcacgcagccaaagacaagtggaattcatggacagagaaagaaaccttccttctttccccactttcaggggaagcagcgactccgaggcgcgggccactcaattgcgtttcaaggcgcgggaggagggggtggactgaggttcctggattggctgcagtgacgcagtcatgccattaggtgtcagcaaaagctcagggcctcggtgggatggggcggctcagcgcttagcccccttccccagccctcttttctccccgatttccagttgcctctggccctgcagggtcgcccaccgcccgcatttcttcatgtacatggttcctcctagactactagggccgccttagcttgctacccttttaggaccctggagctgtgccagggtcccctctgtccccgcgctcctgacaccccctcctcttgcagggccacctcctccccagcccctcctgcagcgtctctgctccggacctcgcctcctcctgctctccctgggcctcagcctcctgctgcttgtggttgtctgtgtgatcggatcccaaagtgggtgccccaggggtgggaagggggcaacattggggggtgttgacgggggaccgtggcaagggagtggtgggtgcagtggtggcggacacagcgatcccgttttcttctctctgcacgctgtcctggccagactcccagctgcaggaggagctgcggggcctgagagagacgttcagcaacttcacagcgagcacggaggcccaggtcaagggcttgagcacccagggaggcaatgtgggaagaaagatgaagtcgctagagtcccagctggagaaacagcagaaggacctgagtgaaggtcagagagggagtgtgtgtgtgtgtgtgtgtgtgtgaaagagagtgagaatgtgtggatgtgtgtgagaaagtgtgagtgtgtgtggatgtgtgtgagaatgagagggagtgtgtgtgtgtgtgagtctgtgtgtgagaatgagggggagtgtgttttgggtgtgtgtatgagagccttgtgtggatgtgagaatgagagggagtgtgtatgtct

SEQ ID NO: 61 - RNA, 461 nt, unidentified
  source 1..461: mol_type = other RNA
ttgaaacaggccaggcagaggccaggctgagtcctccaccctcattgcaggagtccagcgtctggcctggggacttccctcaccagatctgttgaccaacttctagaaacccctgctttggggttgtgaattagaagggctgttttgaccctgaatatcaggcttcccttcctcccaggcatttctttcccgaggaggaaggtttccagtccctctcccaacccccaccctcagcaagccacaaagcctccaccgtcttcagctggttacaggtcaggatccaggtcgggggggaggggcaggtctcctgaggctagacttgaggaatgtttaggccaactcaccaggcagaggaggaaggggccagtttagatcactaagccaattaaaaacaaccagccagaaagaacctttctccttcctgacagctactctgccagctacctcctgggttttgttgg

SEQ ID NO: 62 - RNA, 427 nt, unidentified
  source 1..427: mol_type = other RNA
ctagaggatccttctagccttaatagtgtttgattctctcttcatctcccagagaaactcccagttcctcctcaccccggctcccccatactgaccaatctgcatggcctagtcctggtagtgggatggtctgaagctcccccttcttttcccacagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggag

SEQ ID NO: 63 - RNA, 2470 nt, unidentified
  source 1..2470: mol_type = other RNA
tcccatttaaattatgaggaaaatcctttagaacattttatgttaaaggcctaggagaaccaaattttttctttcttttttttctgagactttgtctaaaaaaaaaaagaggaagtggcggagaaaaaactgaaagctcttcaccactctccatctcccagaaataatttcccagattattttctatgcattttcatatacttcttttatataagtggttcttccacttactatgtgactgagaatattgtttccttttctgtaaaatgtaaataaaaccgaccttatgggattgttttaaaaactaaataaaataattcatgtaaagcgtttagcacggtacactctgaagacatgattttcccaacctttagggcactgactttagtgtatacagttacggaaggctttgttaatttggaagggaaaaattacgttactatttggcagataaaatctctcacatcatgggaatccatggaaggagcaaaggacacatatacaacaaaagagggtcatgcctaaagaaagacttggaaccagtggccaagaggacagagagcagaatctgcctcagtttcttggagaggaggaaatattaacactctggggactaagtccccaggaaataagatgaacgaagttctcctttctcctacctccaggtaatattaagtgctaacatgacaggggattcttttccaattgaatagacttccttggcaaaggaaaggccacagatgattgggaataggtaggtagactgacaacaggatagtggacaagctcccagggtaagatcatggggatggattgcatgctctgttctccagcttatttggccctttctaccttgccctcccagtgccatcatagccatcttctgttactggattgggataccatcaggttagctcttcttaataactgcttaggagcataatttaacacttctcattttatagtgatgcagtatgtttctaacttttaccattttaattatgctttggtataaattatttgtacatttatctttgcatatatgtgaaatgttctgtaagacaaattttctgtcaaagggtatgtgatttttttgttttgtggttttttggtttttggtttttttttgagacagaatcttactctgttgcccaggctggagtgcagtggcacgatctcagctcactgcaacctccacttcccaggttcaagcaattctctgcctcagcctcccaagtagctgggattacaggcgcccacccccacgcctggctaatttttgtatttttagtagagacagggttgcaccatcttggccaggctgttcttgaactcctgaccttgtgatccatccacctcagcctcccaaagtgctgggattacaggcgtgagccaccatgcccgcatgggcatgtgcttttaaaactggtacttatgttaaattgtcttctcagaactgtttttattttttaattgaaatgggatctcgccatattccccagtcttgtctggaactcctgggttcaagcaatcctccggcctcagcctcccaaagtgctggaattacaagcatgagccaccatacccggcccagaattttaaaactactgcagtactttgaacaagcatgggctaattcattttgtaacctaactgtagctctcaactttatgacttttctattagagagagacctaacatataatggggttttacttcattaaattgagaaaacaaatgagagtagataatatttagatttaagtaatagtctaacatcctctctatatccctggttccagctcttaggctgatgaaatgtaatcttttctattattaattaattagcattctttttctttaaaaaaagtcaacgatctatatagttgttcagccatctgtaaatgttattttaacaggctatatttacttgagaattttgctttagtttattgttgacagacattatctgactacttaaacattctcagattaggattctatttgtacaactttggagtgtcagcatgctatgctgagttgccatggcaactatagatattatgtactgtagtctaaatacttattctacataagtccctgtcaaaacatgggaaaacatttctatttaaaaaggtcacatatcatgtcaaatgacggatgtaagagtgacattttaaacagccctaattccctttagctatcaaatttctaatggagctcacgcaatagctatagcttctataacttactcttcctcttctcaatgtttcctcccttattatcataaaaggtctcctaagcatcctcctccacccctccccctcagctctttcttcctaatgattatgaggatctgggtgcagaggcattcacctgtaatcccagctatgtgggaggctgaggttggaggattgcttgcgctcaggagttcaagaacagccggtgcaacatagtgagacccaatctc

SEQ ID NO: 64 - RNA, 358 nt, unidentified
  source 1..358: mol_type = other RNA
ctaaaacacagaataccaatgactcttagactcattcccgggcctccggctgaccacgggaaaggggatcttcaggagcgggtgtctcatggctgggaagaggacggcacaggagggctggggaacccgaattgcagcctgaatggccagtgtcctttctccagaatgcagttctcccttgctcagaagaaatgagtctgccagtgggtacaacatgagatgatacttccacccccattggtgacagccctcacctagctatcaagacagtgttcgacttccttcactccttccaggggccagcatgtggctggccactgtccaccctcaggggttgtgaactgaattaaagggttgc

SEQ ID NO: 65 - RNA, 286 nt, unidentified
  source 1..286: mol_type = other RNA
ggagcacctttctcaaggacaagaaatggtagcaaccaaaaggttggttgaaggaaacaatccttccttctggtggtgtgatcccggcttcctcacagagcccttttttccttctgcccagctccatcccaccagagtttctacccccgctgaggcatctcctgggcacagaccaccacactctaagccggctggcctgggagttagggtcctgcccacctcacctgggagttggggtcctgcccacctggagtttagcacgctggtcgctcactttgctttggtc

SEQ ID NO: 66 - RNA, 266 nt, unidentified
  source 1..266: mol_type = other RNA
catctgtgtgaatgcgcactcgttgttcacctgagaaggtggtataatgaagtgaaaggaagcagaagagagtgttatagttggaaaggtgggaaatcaccccctccatgctgaagggaagatttcaggttccaaatgacacgtttccctcagaatgacttttgctgtaggtatgtgaacctgccaccaccctttgaatttggtggattgatttaacgctgtatctgaacctgactgaggagattccttcctggttcatgaccccg

SEQ ID NO: 67 - RNA, 874 nt, unidentified
  source 1..874: mol_type = other RNA
acagtcagtcccatctacaaatgtaaagtgtccttcgggctcagtgccatctacaaatgtaaagtgtccttcgggctctgtccatcctatgaggcatttggaacatggaggcaggagtgtttttaggtttccttccttaccttcgagctgtgtgtgcagggcagggggctccaatgttcccagggctgaggctctgtccttcttcccccagcccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcgggtgagaccccacccctgtcccaggcaccaaaggcctcctggtgccagatctaatcctgcagaacttctctgtcctccttcccccggctctcagcatcgtcacggtggacccctccttgtccagcacgctgcctcccgcctgctgcgacctcactctctcctgctgtcctgggacctcgtgggcctcctcccgggtccccttcctgctcctcatcctctgtttggccgtctggttgttagagctctccccaggcctcaggaggatgaggaataaatgaaccaccccggtcccccaggctccccttcattcattcaaccagcaagtgttcccagggagctcactgtggatggggctccccatgggagctgcagacacagcagggaggaaagccgcccccgcctcctgagctcacctcgtggtgggagacaaaatgcaaataaatgtgccgcctccaggagtgcaacgtgctgtaaggaacataaaccagagaaagggc

SEQ ID NO: 68 - RNA, 576 nt, unidentified
  source 1..576: mol_type = other RNA
attaaaaccttatggtcttgggatctatggtcttgtgagaaatgtggcacactgagcggtgcaactgatcttaatttagagcttaactctggctcttcagctgtgacgtgcaccgcaggtaacgttcttcagtgtagttttaagttatcttccttcctgaattctttgggagtttgcaagcttggacgtgccgaaatcttgccatgttgtacgttgggctgtgaactcctttgaggactggattttcagcatttctccattgcccagggcttaacaccgcgcctgatacccattactgctcaaaacaatgttttatagaatacaatggaaagggattacgttttctatacatgatcatcatccttttgttttaaggttattacgacctattgccctgctaacaacgagccaatagcaagagtccgacaggccagtgtggcagactatgaagaaactgtaaagaaagcaagagaagcatggaaaatctgggcagatattcctgctccaaaacgaggagaaatagtaagacagattggcgatgccttgcgggagaagatccaagtactaggaagcttg

SEQ ID NO: 69 - RNA, 633 nt, unidentified
  source 1..633: mol_type = other RNA
ccagtgcccaggaaagaagacgggaggcagtagcagagcctgggagagatggcctgaggatgttgtgatggaagactgtatatgatcatatgcactgctgtccagaaatattccttcctgctggactcagagaaccccaggacctctacccaacgtgcctgctccaaagaaggaatgaggttctgcagagctgtggtccgagccgtgatcaggaaggatgcctcgggaagtatagttttccccagcaaactgcccagcagtgcatccgccggctgtggctggctccagtgcagacagagctcagaactcggctcctgcaagacccatcctggctcatctacaccatcgaggatgaagagcagccgctctggcctagacaggatctgtctaatgggagccggagtggctgtcccatcttttccgatgagctcagcgagactcaccaccttggactgggccagctctctgcagctgaagtagaagacatgctggaagcggtccccatacagctggcctctcccccaggcttccttcacctgcctggccagtgttgacttcccaattccagcagccccctgcagtatgactatgcgaggttcttgggtatccaggcctgggccaaataagtctctg

SEQ ID NO: 70 - RNA, 554 nt, unidentified
  source 1..554: mol_type = other RNA
tctgctcctttagcatgttcttcctcaagtatctgctgctgttttccttcaagagatctcgacatccttcctactgactacatttcttgttgttttgagacggagtcttactctgtcgcccaggctggagtgcagtggtctgacatggtgccattgcaccccagcctggacaacaagaacgaaactgtgtctcaaaaaaagaaaaaaaaaaaaaaagcaaacatttggaaatataatgtgactggaaaagtgtggtcagcattctgggcagcaccaatctgacatgacctcatgagggagtaagatgtatttgagaagcagccactggtttgttacaactggagtatgcgcaagaatagctggagattaggctggacaatagtagttataccaatgataatatttttcaagtatatcatgtgccaagaattttacatagattatctcatttaattcttgtgatacactatgacccaggtactgtttttatcctcattttatagatagtgaaactaagttacccactgagttaaatgatttgcccagagtcacag

SEQ ID NO: 71 - RNA, 638 nt, unidentified
  source 1..638: mol_type = other RNA
ggagggctcccagaccctgaggatactgtgctatgaaaagtgttacaacaagacgaagatccccaaggaggacggcgagagcacggacagactcatggggaagggccaggtccagacccctgcccttggggcctgccagggacctctgccctcatggttggatctggacactcccgggatgctgcggcttctctacctgctgcaactgcctcacctcacgggagggtctctctacgcaacttctgagtcgaggtgcataagcagtgtgggggctgtaggcccctgtagctggctgcagagagccaagcgcctcccacacggtacagcacagaaggctcctggtgtcactgggaaaagcccagattcaaaaaggctgggctccggagtcatgcacatcctctctcaggtatccctgtgcagaaaacagtgtccacgggggagaaagaggccaggctggtgtcgcagagcgccaatggcatagtcaccaccactcctccgacttacttcctcctgggatcccacaaacaagccccccagctgggagcctggacctccagggaccagggcagccgccacctacttggcgtgagtcatgaatgcccttcacacagcaatcagattccttcctgtctagccat

SEQ ID NO: 72 - RNA, 569 nt, unidentified
  source 1..569: mol_type = other RNA
tctccgagcctcagtttccccatcttccgaaaggggcctggcatatcctgggcaggcagatggtggatcagaggtagtgaggtgcctacaaggaaatgctcccaacagggtggcttggaggaatcctccttcctcagcactcatgggccgggcactgttctaggccgtgggggtggggatcgagcagtggccacactgacaaagcccctacccttgtggaagtgacattctactgaggccatggacacgtacagtacccaatctcccaccctccacctgccagaatgacagaggctcccccatggagattcctggcatgcccagccccacgtctgggttgagagtagtgactcagaagaaaatgaaggctactgctagcaaccaaatgaggcctttcatgtgtgtttccttcctggcttcctcctgtttttctcacacctctgaacctgctgtcaggtgaaaaatcaaggctactttcccactggtgccaggattcccccaacgccacactgagaacccaggagaagcatcacattatatgagagataagaaatgaagagagggagcag

SEQ ID NO: 73 - RNA, 419 nt, unidentified
  source 1..419: mol_type = other RNA
aaggaatgaaggaaggaaagaaaaaagaagagaaagaagaggaaggaaggaaagaaaaggcaggcaggcaagcacaatgtgtctgcaatggggtgaattgtgagggagggggagaaaagacggggaagaggtcagggtgggagtgacttcaggtagggtctttccggccgcggaacggatgagcgaacgtgggcgccgtgtccaccagagggcgcaacctttccctgcccatcgtctcagagaactacatctcccagcggccctcggagtgcgcggcccttctcaaacgagcgctgaggctcagaggccgctgggacttgtggttgggacggtgagccgctggagggggccgagtaactcattcccaggtcctgaaaaagtgtgcggctgagtcctttcctccttcctcggtttccatg

SEQ ID NO: 74 - RNA, 545 nt, unidentified
  source 1..545: mol_type = other RNA
tttccctcctctggggcttgcttgcaaggcggggttgggtcaaggttggggcagcctctctcttcctccctgcttggtgcctcagtgaccagccagctccctgccccacccagctggccccaggccaccggccgggcctgggactcaacttgccaacttacagcctgagggagggctgggctcttcaaaaacagcctcaaatcatccaggccgctgacagccacagggcaagcgcccagagcccattctttctgtctggcctccatctcatccggctatcccaggatcccaggagtggctgttgatggagcccttcccatggactgggcctttgcacacacattgcaccaaattctcacagcagccgagccaggggcaacacacggaacgtctttcatcctgtggacgggaatcacaacatccagcagtgtcggggctgttgcaagaaactgcccagcacaggacctgggccttcgcagtttaatcacctcgttcctcttcctgcctggagaacggcagggactgtgttgtgctgctggtcacca

SEQ ID NO: 75 - RNA, 621 nt, unidentified
  source 1..621: mol_type = other RNA
ttctacatttacaagtagcccacaggtgatgtccccggtgctggtcaccctaggcagccaggccacttgacggaccttctgtgcctctacactgttgacggcgcgggataattccttatggttgcatagggcactcctgtgcactgcgggaagtttaacagtatcactggtctggactccactagatgccagtgaccccatcactcctcgccagctgtgacagccaaaatgtctcctgacatcgccattgccagatgccctcagggctgcaaaaccgccaggtagagaaccgctaccatgcaggatgctaccatcaaagtgcgcccacagcagcaaccttggagcaggcaggaagagcccgatgcccttggtttgactctactaagagcccctcagttgctgaacctgcacacagtcatctcagtaagttaagatcttcctgaggacaatggcgcaagatcgtcttcagctgttcatagcgaagatgaagatccctttcctcctactgttctttctgtgggaagccgagagccacgcagcatcaaggccgaacatcatcctggtgatggctgacgacctcggcattggagatcctgggtgctatgggaacaaaactatcag

SEQ ID NO: 76 - RNA, 499 nt, unidentified
  source 1..499: mol_type = other RNA
agaaaatgtcctgagctgatgcccacattatcttctttccctcgcctctgctggctccctaagtctctgttggataatgctttgaagctctcctgtcatcacaaagatgaatttagtaaagaaaaaagaaaaggaccaaaagctggatgaagacagtcagtctaacagtaccccactgagcattctctggtcccaccttctgggacaaaaccattccccatgacagttagcattgtgttatcatgatcatagaccacattcctgagctcccatcagctgctcggctggcccttctatcctgatcctagcacctgggctccactcatcccagtgtcctgtctcagaactgcaagagagcgtcatcagtccagtgtactcttgtctgcaaaccaccaaacacagtgtcctgttgatcctgcaattattgaagttttagtggggtacgagccagagcagagaaatcttcctctctctagcatctgtgcccccaaaatgaatg

SEQ ID NO: 77 - RNA, 533 nt, unidentified
  source 1..533: mol_type = other RNA
cctttttctttaatttttgtgggtataacagattcgagcaatgcagaaaggaagtaaaacattatttcactctttcagaatgtgtttggttacggtaaaaacaagtggggtttccacctatttccgattatgaaaaagtttctcatttcattgctgatgtttaagaatggtcattggtattttagtagaaggaacacttgagagttttctgtggctactataatatactaaataacctctttcctccttctcaccccgcaagtcgcccaaaatgtgtggatgaagttgctttccaggaagaagtggttgcagtgctgaaaaaatctttagaaggagcagatcttcctaatctcttgttttacggaccacctggaactggaaaaacatccactattttggcagcagctagagaactctttgggcctgaacttttccgattaagagttcttgagttaaatgcatctgatgaacgtggaatacaagtagttcgagagaaagtgaaaaattttgctcaattaactgtgtcaggaagt

SEQ ID NO: 78 - RNA, 418 nt, unidentified
  source 1..418: mol_type = other RNA
acaaggacttatatttactcagttcatccaatatcaattattataataatcaacagtgtaaattggaaagtaactacgtggaaaccacttctccttctttcccatctaaggtctcagaacaggggaaatgactcacctaaggccttataatgggcctgccccgccatgttctattgcattgtttttctgcaaagcctcatcttgcggggttacactatacatatttttcctctcttttcgttttaccaaagagaaaaatatatacttttttactgttaagaaacattttctcaaataagtacgtatttcattccttactgaaatggttttttcttattgatccattgaaaccatattggctatgttcaacataactagagatatctttggtgttttcctcccttagcttcatatcttg

SEQ ID NO: 79 - RNA, 779 nt, unidentified
  source 1..779: mol_type = other RNA
agtgtgaatgccactgatgtgagcccaacaggaagtgtgacctcattctgcttctttgcctctggttacgcaggctgttttcaaaagaaaacaagagaagttgtaagactgactttcccaagaactatacactcatttataccaggaaacagacaagagaacaagaggctgccgaagacaaaacttctctgcaagcacctgtgcacagcaatgtgcgtgggcatctgtgagaagaagagaatctgtgaagtttgagcaagcggccttcccaagatgtaccgaatatctcaactgatgtcaacaccagtagcaagtaaatgtagaagaactgtgatacgcaattatcttttgaacgattagactccttttagcaatttccttgctttgattttgctgatactcagaccattaaccatctgtcagtagcttgtgcttcttaaatgccagtgttcactttggcaatgattttaacttcatttttttccttgaaagaaatgtggtatttttccttctagtagaaaaaaaaaaatcaccgtttttttttagatgcctgtgcataaatgtgaaaggagattgtttatcagctactattaagatgcaaaactgtttgtatataaaaaatgctgaatgattttgatagctacttgcagaaaataagagcgctgttaaaacggtagtggtttcaagtttcttccttcctcttgttattttctcccaaaaccccacttctaactctccctcacacacccacagttttctccctacaaatcaaccagccc

SEQ ID NO: 80 - RNA, 504 nt, unidentified
  source 1..504: mol_type = other RNA
gatgttaatgactttactttgagatatgatggaaaaatattacaggtacacatggaaaagacatgatcaccaagtgaaaacaatctaaccagaaagctttaacgtctgtcagttaagctgaagctgaaattctgggagcatgacatgctgcagggccaaaaggaatggataattagtattcctctccttcttcctcaccctctccttcaacagaatccacaccaacctcctcataatccttctcaagggcagccatatcttcacgggcctctgaaaactcgccttcctccatcccctcacccacgtaccagtgaacaaaggcacgcttggcatacatcaggtcaaacttgtggtccaggcgagcccaggcctcagcaatggctgtggtgttgctcagcatgcacacagctctctgtaccttggcggttaaggttagtgtaggttgggcgctcgatatcgaggtttctacgacagatgtcatagatggcctcattgtctaccatg

SEQ ID NO: 81 - RNA, 526 nt, unidentified
  source 1..526: mol_type = other RNA
ttcttccggccgctctgaccccggggctgcggctccgagatagctcagggcctggaagtgcatgctgggaccaactacataatccttggtaagaaaaggaggtggccctgacaagtgcaactcgagaaatgcaatgctgtagagtccaaaggtgtctgacttgaacgaaatgatcccactgggggacaatgcaacatatactccagggtcatgttttgtcatgatgcagcaaggagatctgttcctaacttggacttggttgtctaaccctcatctcttctctttattacaaggtgaactggaacttacaccttgcttctcctgttctcaggagacttggactggaactataccaccagctgtctcggatctccggcttgcagacggcacatcatgggacttctcagtctccatcactgcatgagccaattccttataataaatctctgtctctctcattatacatatcttggagaaccctcaggaaccttgactaatacacaagataaattcagttttaattgta

SEQ ID NO: 82 - RNA, 2206 nt, unidentified
  source 1..2206: mol_type = other RNA
gctgctgtagcccctacgtggtgagtagatcgtctccacagaaatcagcggaggcttccacagtccctggttggaatggaaccggcggtgaggatgagaggcgctgaagaccagaagacccagggcgtgaacggggcgtgtcatccctggagtttcagtagaatttcaggcctgtctgcttgagcagcagaggaggagtttctgtgtgcctgtgagagactgtgtgtgtgtgtgtgtgtgtgtgtgtgtgtgagatgaatgaacagggttgtgtacgtgtgtgtgcgttagacaaatgaacagtgttgtgtacctgtgtgaattgtcactgtgactccaggctctaggctctgcagattcctcagccctatcgcctcacagacacttgggatgtgcagcaggtaaaagagagaagccagattcatggaggcgtgaaggatcccagcaagaaaacacagacttgatcttgagacgagatttttgaaattggttgcggcctcaaggcaggctctgagaagactcgataaagagttctcaggaataggcatccctcctggatatttcccccttggaagcgccgaggagccagctctatgtttgtaaccatagcatttctaaagctccagccccaggaacctgctgtgaattagagacagattccaccctaggcatcactgatcttggcctctcttgtattgggaaggtcaacactgttccagtttctctcctactcccatctggttgtgctcggtgaagtaggtgagcagtgctacccttctgttaaaaaaaaaaaaaaaaaaaagatcctgaaagaggaaagaatttttgctacacatctgaaagctaaagtccagttgaactccagaggagaacacagcccaggtaacccctcgtttgcagcctgtgagaccagcctgaggacagcacccagctaaactccaggtccatggaaactgtgagattttatatatttatgttgttttaagctaaattcgtggtaattggttatggtagcaactgaaaattaatgcagtaagaaagtctaaggagatcggaagagactaaagagaaataacagctaaatgcaacgtagattaatgtgtgggatcctggaaaagaaaaaggacattaatgggaaaacatatttgaataagatgtgcaaatttttcgtattgtaccaatgttaatttcctggttttgataattatcctttggttatgtaagataaacgctagaatgaagggtatatgagaaccctctgtactactcttgcaacttttctctaagtctaaaataagtggaaattaaaagttaaaacaaaaaaaaaggaaaattttatctatcccaagaccacaaagttatttttctgtttcttcctccagaatctttattgtttgttttcacacttagacctatgatccatcttgaatttatatctgtgtatgtagtgggatgaagttcaaaatcatttttttcatacagatatccaagtgcactggcaccgtttgttaaaaacactggccttctcttaagctgcagtcagacttttactgtaaagcatgggcccatatatgtgcaggagtctttctacttttccgttggtcctctttgtcaatctttgtgccagcacctccattttaattcctgtagctttatattatatcttgataatctggtaatataagttgaagaacaactttgttcttcaaaatcactttgtctattccaggttctttgcattgctctataattttagaatgagttaattaattttctcaaaacaaacaaaacgccctgctgacattttgttggcagtacattgaatcagtagataaatttagagagaattatctcaaaattgagtcttacagttcatgaacatggaatacatttccatttattgaattcatctttaatttctttccttaaggttttgttgtttcctgtctaaaggtcttgcacatctgttaaatttattcctgttttacattttttatgtttttacaaactgttaaggttttaaaaattttatttttcagtagctttgctttaatataattgttttatacttttaccctttattcagcaataaattatttaatttaaagcttgtggaattaaaaaatatttttatgtacataatcatgtaatctacactatctgcaaataaagatcattttacttccttccaaaaaaaaaaaaaaaa

SEQ ID NO: 83 - RNA, 765 nt, unidentified
  source 1..765: mol_type = other RNA
cgctaggggagggaggtttacaagtgaagagtagttagggaatatggtgactctgaagaatcgagatcagcagtgtccaggaattagaaaaaataaactgctatttccgtgagttccccctctgtgagccatttaggtagtgctgtcctcagtctcttggggcatacttctttcccgatctggcacaaatgacccctcagggcttaaagacttgagaatgacagatgatcaagctctggacccatgttgaaagcagacttctgtgaggagaaagaacagctgtttaccatcttcccagagtcaaattgaacacactggaacagagagattgcattcttacccaaatactcttcgtgattgcctgctttgcggctttgggccgatgcaagctgcaaaatggcgagtcttctaaggtgcctttccttccctgggatcctcatcttagaatgagcagaggcacatcgataagaactggaccttgatttcacaacctcatacaaagccaggcaattcttgagccaaacaaagggggtgtaagctatttcatttattttgataatcctcctcttgcacgggagcattttgctgtctttgtcaaagtgaatgacaacaatttggccaactctctctgagctgcactcaacagtgacaggcaaattatcagaggccctgcacttcttattatcagaggagaagccatcagggggacgcatggaggatacctctcccttgtcactatttgtcagtgggacaagggccactgct

SEQ ID NO: 84 - RNA, 312 nt, unidentified
  source 1..312: mol_type = other RNA
ctgggcgctcggacaccgggcgccggctcttgggtcgtgctctcctgcagcgcacacgctcactttctcacgctcgctccctcccggcgccgctttctgacgggcgaacaagtttatatttaccatgatgtttatttgatgacaccgtgaacaaaatgtgcaaaatgaggccctaccatgttttaaaaaatgttttcatcaaatggatctcagagggacatgcccaaaactcctgctaccagagtgatcctctcttcatctcttttctctagccaataaatcaaagtttcatttctcaaaaaaaaaaaaaaa

SEQ ID NO: 85 - RNA, 384 nt, unidentified
  source 1..384: mol_type = other RNA
agtgaaataaaccagaggtttgaatcatggcttgatacgaaattattggaaagaatatggcgatgacatccacaatgccggtaagaagacgaaattacaagcaggatgaaaaatttgttcaatgaaacacagctattaagtggctgacctaataagtgttatgtataagtgactcatccattgtagcttctgggactcaactttaggatcatcgtcatatgcagcctggagctagccatatttgaagagctaggaaggaggcacagtgattgacaatctcctttctcctcaactagaatgtcatttccttttttaaaactaatttaaatcccatctacattcaattgcttgtctaaaataaaacccaactcctctcttaaagac

SEQ ID NO: 86 - RNA, 2261 nt, unidentified
  source 1..2261: mol_type = other RNA
cgctgggaagagactgtgcccctgcagctcccctgtcaccggctccaaggagcgtcgggctccccccgcccagccctgcagcacccatccggcaacgccagactcggcgcaacgggggcagctgcgactttaaatctctcagatccgcggcctgagggctgccgccaccgagaaatggaggcacagagctgtgaacaagagaccacggctcgccgaaatggcggtgccaggagcctgaaagggaatgcagccggcggggttgtcaaggacaacatttgttttggcgcaaccagcggtgccgtcaccaagaaaccgtcgactctgagaaaaaaagagaagttcggctaccgagaaactccgtgcagcaagtgctgtgacagcaaaaccgccggcttcgcgccgctggcgaaagagccaacggaaacgccgggtgctgcgaccgcgaagccggcactagagggcctcggatggagaaagccccgcaccgagacgaggaaactgtgcacagcacgactagcagttgacacaaacagaaaagtgtctcgtctgctctctgggagaaagcgtggatcaaaaccagcaactcagtggagagaaccccgcagccactgaagaagttgcccacgtggcggtggggccagagaaacaccgcgatttcgacgacagccgtagggataccacagagaaacatgcgcggccacagaagtacatccagctccgcgcaatcagtggttctgcgatccagaaaccgccagctgggctagactagaaacttctaaaaaactgtcgtcccatagcagctttcctaaccacgacagacagtcgcctttagagagcacctaacggtgctgagaccggcctgggccagcaaaagcgcagagcggtgccagtgccaagaaacgcccgacttggtgaaaccaaccttgtgaccaccgattccactggcttcgcaggacgcaacgggcgaaggcgcggcggagaaaccgcaggctcccttcaccgattatgctgcgggctggggatggtggggggcacccgcgaaattgtgaaaccagcggcgctgggacccagcgatcagctgcccgtagcaaatgtctgtgcagttgcaaaagataatttttggccgtgagaaggttgccgccagagagcgtccgataacgctgacaaaggcgtggcgttgccagtgagaaacagccggcgctgggaaaggaacggtgcagcgaccgaccaactgcgcgctcggtgccaccaaagtgctactggctctacccactctgtcccccacgggcttagggaagtctgatgtctttttcttttcttttttcctctgtcgcctaggctggtgcagtggtgtcatcttggctcagtgcagcctcgacttcctgggatccagcgatcctcccgcttcagcctctagagtagccgagaccacaggtcacccctcgctgccaggctctcttcccggtccagagcccacaggatcctacaggaggggccaacaactgcttgcctttgaaacttgaaactctcggtctaaggttccttaggagcgtaaaaggcacagcgttttctgatcgcagcttcaggtctcccgcccctgtcccggtacctctcctgcaggacggaactctgtgggaacgctcgttgattctgatggttaactgtcagatatccttgatattggacataggatttggaagagggcaggagagaaaaatgaactgcaagactccagcacaagaggtgggattgctggcagatgtctgctccctctccaaacctaatgaacatctcaaagtgccatccactttcctatacccttgatctgtgaaatggatcactgacgctttctctgtctttccggaaatgcaaaacaggtgaattttcaagcgcttgaattgtctctcacacttttcagtcaagagtggggctataaactccttcctcataggaaataaggaactgccactgcttggaagtaaaacgtatttttcccataagctttcacatttcccaaaaaaaattatacatccagatgtaatccccctaagaggcttacagaccctaccaggagcgttcccacgcgaacgcatcatctcccaatcggatcctgaaaacaccatgcaaccaattccatccttttctggatcaacctgggcagaggacaggtgcagaggagcccagagaagggccttgacaagtcaggagacccaatttggggtcgcaattgtcactcactccccaggcgtttgctttgatca

SEQ ID NO: 87 - RNA, 1012 nt, unidentified
  source 1..1012: mol_type = other RNA
ggcatcggttctcctcactctccagcctgttggctcatcctgtggattttggacttgccagttccaacatggttcaagccaattccttatcacaaatctctgtctgtatatcagcacatcctgtgggttcagcttctcttgagaacactgaccaatccaatcaccgttactcccgatgacttgtaggtgaagccccttaaggaaaatttctttcacaaatgagagctagggaaagatttactcttcctgagcagctaaagtattttctccaatggccacgagaaccttaaacctttccttctttccacgctcccattggccgccaggaacttgcagcttggctacagcaatcatgaaggacctgactgggctcagcacatgtgcccaggccacccaggtagaggtggagggcatgggattcaagcccaggccatcgttggctctgaacatatacctgaaacaacctccctaccaggaaggaggctgccctgcctggagtgaagagtgcatggagcagtctcagccgacccaggtgggatgcgtaacatggccgagaaatccacccatgctgctgagagctactgcgccatggggtcatgtgtcacctaactgacttagcccagcctgactgatcccccgtgtgtgaccagacatcagcacattcagaggacctcatactgagaattggtggacctttcagaatggacatgaccactcaaagtagggacattactcgctatttgatggcccatgtgggatcaaaggccactggggttccctcaaggcacagcacacttagaatcccataagtcctcagttctaaggcatgtatttttcatactttgataattctgaaatcaagtatagcttctagtagatattaaaactcattttcagatcctgcagaaaggaggagacagaaataaagttgccattccctgcttttgtatgaacccagctgagcataaaccgtaatctattcaacctcaaaaaaaaaaaaaaaaaaaaaaaa

SEQ ID NO: 88 - RNA, 686 nt, unidentified
  source 1..686: mol_type = other RNA
ttcactgctgtacacccagcccccagcacagcgcctgtccaggacaagtgcccagtaaacacttgggaagcaatgcaagcgtgcgtgcatggataagtatttctttggcagatgagggggctaaggttcagagaaggccctgggggtctcagactcatagcccagtgctctttctgctgacacgccctggtctctggggcagtttgttgcctgttcagcaacaaagagggtgtgcctcgttaggggtcctgcgtgcgaatcgcagtccctgcgtgtcttggctggaggtcaccaccctctctgctccagggcctgtaattaccacttaccctggtcaatgggtccgagagattacccttgtaggcagggctgtggccagggtgctcacctggcccccagcaggtcccatgggcactgtctggccgggcttcatggctgacattccaggtcctcccagcagctcctgcaaacagacccccgacccaagcccttccttctgcctccactgccaccactgctgctcatctctgctggcacagaagtctcttccctggtcttccagaaatcccctctccacactcagccagagggagctattaaaactgtgggccagcccacatcagtccacagcaaagtcctctctaagggatctctgttgcttggagaataaaccctcggattccttc

SEQ ID NO: 89 - RNA, 677 nt, unidentified
  source 1..677: mol_type = other RNA
gcatcgactcacgcgttcacctccttccggtctaaatgttgtctcgcttctcagaaaggaaacactggttctgtaaacctgtttgagaagaaaagctcgaggtttcttggcctctctgcttctggagccaggggaaaaaatagccccaggctgaagacaggagggaccagattgctgcttggtgatgcgatctcactgcagaggcagccaggagaccctcctgagacgaatcggagagtccatgctgcttttacacacatcctagttcttttataactagccttttcctattaacggacatcattcaaaagccagttttaagcaacataaatattgaaaacaatactatactatttagaattaattataagttttgtttttaaagcctaaatcttttattgttgtttgaaccctgggcactgaagctaataatgtaacttttctcccttctttcttctttggcatagatttaaagaactgttagactcatttaaaatgctattaggcattttaaagagtaattgttaaatgtgttgtctcttgtgggactggaaatgttctgttcttatctggcgctggttacatcctgtttttcatttgggaaagtgcattgagctggaaatttatatgttcaatttctgtatgtaaattgtacttcagtaaaaattttcaaaaaa

SEQ ID NO: 90 - RNA, 567 nt, unidentified
  source 1..567: mol_type = other RNA
agcgagactccatctcaaaaaaaaaaaaaaaaaaaaagaaaagaaaagaaactggcggacaaaggaagagatcctccttcaggcagggtagggaagaagggcactcggtgaccagagaccttttccaggaccatgtctttgaagcaaatctcagctcttgtcttccggaagcgatggagacagaatccagaagcctgagactctctcctttcctggctttgatgaagcaagctgccacactggagaggcctacatgataaggacctgaaggtggcctccagctagctccagccaacagttggcaaggaattgaggacctgagtccaatagaccttgaggaactgaatcctgcaacagctacacaagcttttgtgatgccctataattacatgaactctactaaaaagagggatactggaaagaagaataatgttgagacacctttgcagaagaaaaaacaacttcctctcttcctctacttcactctggcaagttctccaacctgacaagccagtagggagtgccggaatgtcatggctgagaaaagagatgttaacaggctgtatg

SEQ ID NO: 91 - RNA, 551 nt, unidentified
  source 1..551: mol_type = other RNA
ccccaccaaaacctctaataaggcacacagctctgctgacatcccatttcagtgttttgagaccctaagcagcagacgacccagttcagcccacctggatttctggcccacagaaactcaagaatcaaaattcactttgccccatgatcttcaccagaacggcatacttgcctctcccctcgtcagattgaaaaggttcctatgatttagggtcttccagcacccctcaggaagcttgacaatgaagaggaattattgatgaatgtggaaaggccagggggtgcacattcatttttaatgactacacaccttcccttgactaaatggaatcaatccttcctgcctgaatttcatccattcatcatgctctcacagtggctgacacacagtaggaactcaatatttatttgttggataaatttttaaaaagatataacttacttgtatcactaaattttgtatatgaagtgaaatatggctgtctgagaaaataggacaattgagtttatggaagaaaaaagtgcagataaagattaaaggtcaaatacttc

SEQ ID NO: 92 - RNA, 384 nt, unidentified
  source 1..384: mol_type = other RNA
gaagctcaagttcaccatgagccacagcagtaagccatgctttagcctctgacctgtgctccaattttggtacaatgatttctgccatgttctccaacagggtgaggaaacagacagcactaagagaaaaagctggaactcaaaaccaaattaactccaaacttaagttttcccacttcgacaagaaaaggaactgcatgtgtttttctgcctcttgcagatgtatgtcataaaagttggatgccttcctttcaagacatttgaatggatcaccaaataattacctacttttctctcacaactcagataaataattaaaaggagatatacacagattggttgcatacttaagagtgagaatcaattaaaacaaaattgtatgca

SEQ ID NO: 93 - RNA, 620 nt, unidentified
  source 1..620: mol_type = other RNA
atccagtccccttccttcggtgtttgagaccacttcatctggaccgagctaaagtctaggaagaaataaagtttcaaacccagtagagttacctcaaagatacacttgagacccttttcagaagatggcaccgaaagtgaagaaggaagctcctggcccgcctaaagctgaagccaaagcaaaggctttaaaggccaagaaggtagtgttgaaaggtgtccacggccacaaaaaaaagaagatccgcatgtcacccaccttccagcggcccaagacactgagactctggaggccgcccagatatcctcggaagaccacccccaggagaaacaagcttgaccactatgctatcatcaagtttcctctgaccactgagtttgccatgaagaagataaaagacaacaacacccttgtgttcactgtggatgttaaagccaacaagcaccagatcaaacaggctgtgaagaagctctgtgacattgatggggccaaggtcaacaccctgatggagagatgaaggcatatgttccactggctcctgattatgatgctttggatgttgccaacaaaattgggatcatccaaactgagtccatctggctaattctaaatatacatat

SEQ ID NO: 94 - RNA, 1651 nt, unidentified
  source 1..1651: mol_type = other RNA
aggcaacgggggcgggtccctgggtctgcagagcctgggggccagaacacgtggcttcccggtggccctcaccttgcagagttggacaaagaggactaagttggtggcctaggtgtggaggctggtggggccaccaccacagaccacaggaggggatgtgtgcaggccagggtccagccttgcctgagggttgcacagtgttttaagtgacttctgcactggtgggcctctcctcccgaaaccagtctctccctcttaactccatacagtaagcaaggcccgggaagaagttgggtttccctgccatccttcctgccccagcccctcatcctcaactcaccatcaacccttccacgatcccaaatcgctcgtggatgactttggcgaggggagccaaacagttggtggtgcaggacgcgttgctgaaacagccaaggtgtgcacggacactgtgaggtggtgacctccccaactcagccctcaacctcctcagaggcgccaatgctgactgctgtcagccagcccagaccctttctccatatgctgcccgggacgtggactctgtgactgtgaggcctgcccggctgctttctgcttcccagctctgacaacctcgccatgaaccctcctagaccctcagctcctgcaataggaagctggctgccaaaggccaggagggtccctgaaaacagacactgcattaaagaaaaagctcctggaggactcctgaagcctgaggcagccttggggcagcagtggctcatggtttacattggaaaggtttatcatgagtaaccgggtgctttgcttctgtgattcttggccagtgtcggtctgtttcctttgccattgagcctgctcccctcaatggggggttcaatcaatgtttgtggggtttgtttgttttgagacagggtcttcctctgtcgcccaggctggagtgcagtggtgcaatcagagctcactgcagccttgatcttcctggctccggccatcctcctgccttggcctcccgagtagctgggactacaggcgcacaccaccatgccaagctaatttttaaatttttttgtagagatggggtctcacttttgttgcccagggtgctttagaacccctgggctcaagtgatccttgacctcccacagtgctgggattacaggtgtgagccaccgtgcctggcctcatgggttttctttgttttgttttgttttgtttttgagacagagtctcactctgtcactcaggctggagtgcagtggcacgattttggctcactgcaacctccaccttccgggttcaagcgattctcctgcctccgcctcccgagtagttggggttacaggcatgcgctaccacacctggctagttttttattgttgttgttttttgtatttttagtggtgacggggtttgctctgttggccaggctggtctcaaactcctgacctcaggtgatccccacttcagcctcccaaagtgctgggatgacaggcatgagcctctgcacctggcctcgtgttttttttttagttagccattttatacgtgtttactgaacacctgtcctgtgcccatattctaagtgccttacctgcattaaactcatgtaaccttcacaacccgaaaaaaaaaaaaaaaaaaaaaaaaa

SEQ ID NO: 95 - RNA, 1954 nt, unidentified
  source 1..1954: mol_type = other RNA
ggcttttattacattaagggatcatggcacaagttgcagtttccaccctgcccatggaagatgaggagtccatggaagatgaggagtctgttgaagatgattccgtggagagcaggatggtggtgacatttctcatatcagctctcgagttcacgatgttggaatttctgatgaatctgcagtcaggtaagatttcatagatttaaaaaattatgttaactaagaaaatatagatggaagaaactaatatctgttgagtgttgtattctgggctagacatcctaatatgttctatgcatttatcatctcataaagccatcataacatctgtgttcctataacctactgttaaataaacaactatggattagagctgattaattgcctcatgatcccatagttaacaaagtagctggcctacagtttgaccatcagcctgtctgccttccaaatcctttctcttgctcctcagcatagattgatagatatctgtgcagcccttggatcaaagtataggtctgaatcagatcaatcagattcattaatttgattaacttctaaattaatgagagtttaaataccttgaactctcatttaagtttattgttagaatgtggttagtcctaataaatttgacgatttcagtggtaaccagtatcttatttttaccttcaaaggctctagggcagatctgacttagctttggccataggactgtaagttttaccaaagcaagtttaggcaagtcttagagacaaattatttgacttcccagtttggttttccatttaggcaagtatttctgcttacttccataatacattttttagtcttgttgctttttccatgacttttatataatcttgtcttcattttttaaaactttcttctctgtttttcttggtgtttcttttgttctattattttttcaaactctgctgcccatgtattccaagtttttctatagacagaatcaagaggacatagaattacagaattttaaggaatcttggaatgaattaaaataccttctagtatttttctctgcgttgaacattctggtcaagtgattctctagatagagaatgtgaggctcaaagagattacgaagcttttttttagacataggaattggcagaaatgagatttgaactcatggtaaagcccagtactcttgcttctttttatgtcctattggcgtgtgttttcataatacaaacgggagtgagtctgtggatagaatgagaatggaattagctggtgaacccaatggaagtagataagaatggaatgagcaggggcagtccaagtttgaagataaacaacactggattggataggagtacggactcttctacaagagatcaaagtattggggtttatgacaagtttgataaagataaattataaaaatgaaggacacaagatgttgggaattatctacaaaggcacattaaaatagaaggttcaagggagctctaaaaattttgctgcttttttttttttaaatcaaggactgacaaacttgaagatttttactgaaagatgccaaaacatattgagacactgggaagaatgtctacagcagatagaaatgtggtgtcatctacttccatcctgacttacaaaggggtggcttagagcccctggagtactaaggggctggaaattgctgaactacatagatgtgtggcacagggcaggtgtctcctcacctctgcctcttttcacagttcactgatgtccttcccatgtccatgtgggctgggtcaggggcatgattagctggcaaatcagtcatggagttcagttgggtagttggtagtgtgtatagctggtggcaggtgatggagactccagttagcttgtttttcaggagcagggatatagagagctcctactcctggtcatttgaggccattctttcaggaatctgtgctttcatacactgaag

SEQ ID NO: 96 - RNA, 11406 nt, unidentified
  source 1..11406: mol_type = other RNA
gtcaaagtcctttaaatgctcaaactgcctgctttggattcttattgaacttatactccaccctgtggtacaggggctaatatgctgggcctttaccacttcagcccaggttcgattcctggtcagggaaccagtgccacttgtcttaatttgcatgactctttaccctttgagaggtgaacccagaaaatccgtgacaggtctcagttaatttagaaagtttattttgccaaggttgaggatgcacccgtgatggtctcaggaagtcctgacgacatgtgcccaaggtggttgggacacagcttgcttttttttttttttttttgagacggcgtctcacactatcacccgggctggagtgcagtggcgcgatctcagttcactgcaacctctgcctcccagactcaagcaattctcatgcctcagcctcctgaaaggctgggattacaggcacccaccaccatgcccagataatttttttttttttttgagacggcatctcgctctgtcgcccaggctggagtgcagtggtgcgatctcggctcactgcaagctccacctcccgggttcacaccattgtcctgcctcagcctcccaggtagctgggactacaggcgcccgccaccacgcctggctaattttttgtactttttagtagagatggagtttctatgtgttagccaggatggtctcgatctcctgacctcgtgatccaccagcctcggcctcccacagtgctgggattacaggcgtgagccactgcgcctggcccacagcttgcttttatacattttaaggagacatgagacatcaatcaatatatgtaagaagtacattagttctatccagaaaggtggggacagctcaaagcaagcccccaccccccgggggaggtagatggaggctgagagatcagaccttgcggggcacttggggacatcaggtgggacaggccccaggtgcaccctcagtgcactgggcaggtctcaggatcctggcctggtaatgtggtcactccctcgaggactgcttttggggcccagtcacccaccctgggccatacacgtcctttctcaggactggactttctcagctcctgcagagggcactgcctctagcccaggaggggcagccctgttgtgtagcccaagcccatgctctacagggccctagtggtccctgccccacccacacacccctcttctcccaggtcctgtcccttcacaggcgtcagcccacagggagaccctcttcctaccttccctgtgtgtctcctgggctaagaccaggggtggggtggggagagacacggctggtccttccctaggactgctagggaaggaggaggacagctcccggcccagcacaggccctcagctctgtctgggttgccttacagtgagacaaagctgccatcgatcagtgccctggaggcaaaggtaagacctgctgcctgagaggctggtccagggacaggaggacttggcaggtggtcagcagcccgtgggcaccagcgatggtgagtgggagagacaggctgtccctgggaggggtggcagttcctgctggacctgaccccaagactctgtccccattggttgtttgaccaaactccaaactgagaaccgcagtcacggctcgggggttgctgtctgcttgcaccaggaccccacctggaggctagtacttgaccaaagtccagtgtctgtggcctgaggatggcatgtcccaggtcgtgaggccaggtcacagacgcctgttcagtcagagggtctcagtccccggggcctgccctttcctggttctttttgtggttcagtcccgtcagggcctcggagcccaggatacagcatccaaaggctgcttcaggaagccttggtagctccgctaacttctggtcatagttgacagcaaagacgtcaggagatgagaggcacagagaaatggctccagatgcttcaagacgggcagggatataggagtggcaaggaggtgacatggggagggagggcccccgtgaccgctctctacagagtcagggggatgggctcccatggctgggtcctggcactgcctgcctctcagagggtgggtgacacactgtcctcaccctggacagcaggtctggtcaccagctttcctgtttgtccctgcaaacctcaacttgctggagacagtgtgatgccagggcatgagccaggactatctcagggatagggacaccccctggcaaaccgaaggaaggaataggtcaagatgagaattcataactctgaatcgtgggagggaggcctggtcctaggtaaactgcacatcccctgaaaaaccaacaagtcagagactggagacttccagaatcctcaggctccgatcctccctggggctgccccagaccctgtttcttcagagtgttctgtcccaagttgatagacagcgtgtataaaggcattctccatagatgtggggccaagtgtggtcacacttcctggatgtcgagaaggtgaaggccgagaactggtggaaatacgaggtatgacccttccgaagtcagccagaggaggacaaacaggccaggctgtgttgagagcccaagcctccagctggagggaacgcagagcctgggttgaaggtggagggggcatggtcaggcggacatcctgggcagagacggtgatgccagcaccaaaggtgagctgagctccaggactagttggattgtaaggaaccaagccagaggaggagctttctgcagaaggaaaccaaccttccttccttccttccttcctcctgtcctttcctttcctttttcctttcctttttcctttcctttcctttcctttttcctttcctttcctttcctttcctttcctttcctttcctttcctttcctttcctttcctttcctttcctttcctttcctttcctttcctttcctttccttctttccttccttccttccgggctgcctgtggacgactgccgtgtggggcagccagtcttctctctgttcagaggctgcttcctcttcctggccctgccctggaggtcatgaagatgaagggaaagaggtcccccctggaacttccaccagatcaacctggatgtcagccatacactaagaaaccacatgatgttttggccacaatgcagtgtcaattaaggcttgcagggctggagagccctggggagatggcgtgaaccagagggatgggagcttcaccagagcagaggcaggggtcacccaggaggggtgacagagctaccaaaagctttccaggcccagggagcagctagcacaatagactgagtgccttcctggttgcaagccctgggcctggctgggatatgtggggccggagcccaggtggctcccgaggagacagagagcaatgaacaaaatcatgagaaatggtggaaagttcagtaacctacgggagggcggcaggatgaggggctcgggagccttcccaagccacactgacgacaccaaatgccgagacgatgtgcggtcccggggacctttgtccctgccaggtgggaatgcgacagggcacagcccctttgggaacccgttgggcggctctgcacgaaactcggtggactcgtgccacatgtcccagaagattctcagagactgatcccactgatttgaaaactgatggccatgtaaaacctgcatgccacgtccacagcttgattcaccgtcactcacagttgaaggccaccaagacaatcttcaacccgggaatagtaaaatccactttcaccaaaacatttcataaaactcagtggccctgaagagctcactgcttattctgcgcatcatctggacctggaattggattccttgttggaacacgatagagagaacaaaacagcatccgtgtgaacccaaaccctcttttctctctttcctaggctgcagcatgtctttcttgtcctggtggcatattgcacctaatcccatgagtattccaaggcaacaatattcgcgggtcacattcccatattcataggcacgggtgtcttgggtagggggtgtcattgcttctttaaaattcagtatctgacacctaccaagacgcagccggtaggactcaagctcgctgttggcttgaacattcaagcgaggcagggagggagggtctcagaggggtcatgctgagatggagagggctcaggcacagactcctggtctgacctgagcctgacccccaatctcagaacaagaaatgacaccctcctcctgggcttgccccaaggtccaggagctggtcagtgtcccaaagtggtcctcccaggaggcatttttgacaagttgccaaagtacccgatggacgtcgctcttgtcacgaaatgagtgtggatcccgcggaagctgttccttcattggaaaactggcagggatccaatgtcccctccgcccgaaccccactggagcgcagcagacagggagggggttcctgaagtgcctgctcccactgtgcccccagtttccagatcactcctggagggacagccactgagctggctccatgggctgcacacacagggtctggacccagcctccctctcatacctggcaggcagcccctgggtcttcccaggaccctgtatgcggtgggcccccagcccgccctgttggccagcctagctcacagatgctgtcccaagcctggcacggtggcctccctggcctggcatctgagggggagggacatccacggtcctctcctatgtcctctcgggccatgtgggcttggacactcgctacaggaatctctctggactccttgtcaactgagacgtgcgtgacttgctcagtcctgacacccagtgtcaggagaggggtgaacaggctgcaccccaaatcccacagggcctgtggcaggcctcctcaatgacatcagtcctctcaggtgacctcagccctcccaggtgacatccttccatggtgactctgactcttgcaggagctgggctactgcagggccatgagctgcattgctgccactctcctctatctgcccagaaaagataccttctgagtggtggcccagctgctggctggtgagaggcactccttgcagggtaggtggacagctgcccatggggactcacacagccaggcctggggatggtcaccctggctaggtgatctcagcttccaggcaaggagccttccttgtgtccccagctcgtttggagcctctaggatgttgcctgctgtgggtcatgcaggagcctgggactggatagggacccccacaccctgagttagatgcctctcatccccatcagcagagggcatcacagcctccctgtggctaccttctgtatcccggaggcttggtcctccaaccccgactttgcgcaggcaacgctcacctccctgagggtcctcctgcctcctggtggctgactgtgctcctagccgtcctcgtcaccctcagacaggcagatgaaacccagacagtaatgcttcctcatcgcatgtcccctagcctgaccctatgaatgtccagaagacagtaatgcacccccagcacccaccccgctcaccctgctggcccctgcctgccacgttcctccctcttggcctccacggcaggcctctcctcctaatacctcagctcagggcccccactgggcagctcctcccgccaggatcccggggacattcctgcccctcctcccctgggccaaggctgcatgctggaggggccagatgggagggtgggagacctgggggtctgaggacccctgccagtgccagctcttccagctgacggctccacgtcttgggagcaggctttgattttgtgataggttgtgggctctcaggattctgcagttcaaattgtgccaggctccagagactctaagcccacccgggacacgtgttatggacatcatccccgaagcttatgaggacttggtgaggagacggtccccttggctcttccccagaggcttggggatcaggggtagagggaggttctggctcctcctcatagggggtgagaggttgagtcccagcaggggccagacctgggatgtggggttctccatgggattgtagttgggtttcctttttctgctctggagcaggcagaggctctgggacggggactgggctctggctgagctgggctaagggaagtgtgtccaccgaaggtccatgcggggggcaggtgtggcggggtgccctggggaccgcccagtgttctgtccctcagggaagggctgcagaggggcctgaaatagggatgttttgggggcagcccaaggggccctgagcacctctgctcctcccatcaggacaaggatgagctgcatgcccagaattccacgtttacactggctcctctggtgctttaaatgggggtaatgaggcactggaagctcccagtccagggaccctcctgccccgcactgcttggctcccccagcccaggggtccggcttccccaggaggacctggctcacccccacccagcaggaggcacaggcgggtccccacagggcacacaagccaggcaacacctgagagggggcatcccacagcagaggccagggcagaacgggccaggtctccacttggaagagcccggggaagtccaaggccctggggagcccctccctccaggagctacatccctgctgagatgagtcccctccatgaggagctgcaagacctcttctgacccagcctcatggtggcagctcatcccatggggacgggtccctggcactccaggggactgaaaccacagtgggccctgcttgggccaccagctcccagcctggaagggccaggtcctctcacacctgctgtccccacagatccccttcgggttcagcctgaagatgaagcatgcaataatggcaatggcgcccacggccttcgaggttcacagaagcaagtccccctgtgtctgaggtttcctggggaactcatgggacacagcctggctggcctgaggcctctgtccccatgaccttctgctctcatcagaagggatggccagggatggccaggcctggcctgtagggctgggcagggcatagtgggttctcccagaccctgagcccacccttgaaatgagtgatccatgtgaaaggtgggagtgtggtgggaacctccctgcccgggcctcccccggccatggcctcctgtgcacagctggaccccagggttggccacagaaggacccagtgctgctcagtgggaggtggcagtgttggaaacggggtgtgggcaccaaccccttccaggaacccctcccggcctgatgcccttcctgtcccttgagcgaccatgaacctgtcccagtccaccctgtgggagggagttctttcaggaccggtgctcccaggcctgggccctggaggatgccacagtgctcaggcactttgaggcctttgtgggggaactgatgagccagtgctgggatctgccaccccctcgtgggctccagcaccaggtcccctctcctaagtcatcctctggagcattcgatagtggagagggccccggccccaccagccctactacctggcccttcctcctgcacctctttttctgtcttttcctcctctacactaagcagcttctgggcaggtgctgagcgcatatgtactggacatgctgctcagacaggcagggactggagaaagaaccctcaacgccccctatgctttttacatcctctctcctctgtgcgagctccctaaccaccatcccaacatcgacagatgctgagaggagcaggggccgctcactccagaatccccctccaaggacgtcagggcaataagccttgagccaggggaaaaagagaatcagtgtcactgacccagaggacttggggagaggacacggataggagccctggccagagccagatccaagagttcagccaggtgcaggtgtgggaatggtcccgtccaggcatggagtggacggtcctggagggcaaagggggccccgtgcctgggaccatctcacccactgtggagatagtgtcttgtgtgaggtggcaagggggctaggtgacagccaaggcctctcccacctggttctgaattagggctgctacccaggccacacagccctgggatgaggaggtgaaggaggaagtctggagccagcctgaatcctaagccacagccccaggagtgacgtgaggtgccccaacagctccccgtgccctgagggatccacacacctcttgctctgtgatcagcagcctgcaggggtgtcctcagagtcagactcaagaagccacacagggatgctgaggacaccagagacccaggtctgtggggcccagccttgtgaggacttaatggacttggaggagaagctgaccgctcgcatctgctattttttcagccaaacccaatcgagggtccttggcacccaggcctctgtgagcataccctggcattgagcccctctgcaagggagatgggctggcccgcccctgcctaccagcttagctccaggggtccatgcagccagccccctaccccggaatgcaaccctggtcctctcccaccctgacctggctggcagtctccaagactgaggcccaggggcacgaaggagcttccagaaagctcagggcagcaggttgctctctggggatcctgagggttcagaatggacccatgcattttctgaactccctcccgaagctgcccagagacttggctgtcagaggcccgtggttcccattgtgcaattttgaacagggatgctgtgctccgtcccttccacatccaccctacacagccccaggaccatcagtggggcccagccctgcaaccccatcttggccacaacaccaggccctgctgggccccgtgagaactgagaggcaggtccagagataagtcccttgcatgggggcacttgcatcggacccctcttggtgggcaatgactagcatggcatggaaggagccagggcagctcccagccagttgctctgcagccccagatggctcctgggccttatgaagtccctctcaacagggaagctggtccctttcaggctgccagagggaggggtgaacgtggtgtcctcccagccctggccagccaggagcatgccctgtggcctctgatttgacttgttggacagagtcccctcccagggcacaagtcctaggtccagaggggcccggcctcaaccccagccctgcttggtaggggagggcaccggggtgccatgggcctttagctgcagcaccgatggccacgcaggggacatggcacacctcacccggttgttgtaccatttcaggtcagagaaccccggcaagaacttgctgcaggaacacgaggtgctgagcctgtgcgggagcaggacatcaagggctgctcaagagcgtccatggctacagtgactgtggcctcttcccagcctcatctgtgccgtgcaggtgatctatgactccaagctgggactggcaggtgatggtggccccagcatctcagccacgctgccaacatgctgtcaggaggcttcgaggccatgcccggtatgctgcccacctccttgagaccccttcctgacactttccagccggtgctacagccgcagcacgtgccaccactgaatacctttcagcagccggtgcaggcttcagacggcaggcgtgcccgacagccgtggcctcagcagctctttcgtggcaaccaggacagtcagggcagtgatgaagtgatgctacctgagaagtgggccacgagctgcttccaggaggacttcatctccaagcacaggtagagcacgatctgcgggatgagctacacggccggccgcccagtgctggcgcagtgccacgtcctccagcacttcctgatgtgcctcagcagcaccgagagaaggcctggaaacaaggcaagaggaaggccttctgtatggcacagcacctcttgaggaaagaaaataagaaaactgcctccgatccaagcagagcaggtcagcgagaaggtgggcatcacttgggggagtggatgtgggtatcgaagacgaacactggatacgtggcctggtcatgggaggtgggtggtgcgagaaaggtgacatggtgggtgccagctcgttcctgaccctgagcactgcaccaccactgccctcgacctgcaggaggtggagagcatgattgacagcttcatatgctttctcaagaaggtggatgacggcgctgctgctcagccacagggcatggagttcacggcaagcaggtgaggggctttgagaaggagcatcggcagggggccagccctcccgcggcctcagccaggctttggagtgggcccagcaggccttctgggtgggcctgaaccagcccatcaaccttcaccggaggtgcctacaataccatcgtcaagctcttcccagattggcccagacaggacctggattctgtctcggacctgttactgctgtccaagcagcaacgcatcaaattcccagacatcctccacgttcacagaggagctcttaccaaagtcacggggagcaggagactcgtgggaggaggaatgatggaggtgcgggggctgatggcgttcaggagcgtgggacacgagttctttggccactttggctgaaattcaccatctccatccaattcgagggagaggtttgagatcatagatgcagcactttttaccagagcaatacatatttttccaaaaatcgatgtagaatttgaaagcagctctttataaagatgagagtgttgagtgactggatgttggattggggactttttccagttcaaggataatttccacagcagaataaaaactgctatcaaagagccatggccaactatttgtggtagtacaaggatggttttgtgctcaactgaggccagtttagaatagagaattatgtgggaaataaacagagaacatggcgatggaaccacgtgttgtaacttcgtaggcatccatactatggaagttccttccttctctccttcctgctgtctgtgcctcctcccagcatcgggcatctccatgtcctggacctccagtgaccagtcctcactgtccaggacccacggatgaggcttccactgccaggcctccactgactgggcccaactgacaaggccccctctgtcaacgtctccactgactgggccttaaatgacgaggttctgactgaccaggtccttactgaacagacctttactgaccaggccccactaatgaggactccaaagaccaggcccccacagacaagaccacagttgattatgctcctggtggccacgtttcgctgactaggccccactgacaagtccctcactccctaggtccccattgaccaggccccaaatgatgaggcctcagcttactaggaccccactgaccaga

SEQ ID NO: 97 - RNA, 1054 nt, unidentified
  source 1..1054: mol_type = other RNA
atggagcccgcactctccagttcatcaccaccccagcatccctactcttgcatctaacagtttccgctattttgcaccacctgcctggcccttatgggcaactcaaggaagaaaggaaagaagagatagaggaaaaatggattcaacaaatgaaagtgttctttctgactactgctgtgtttacaaacattttaatcatcaaaacatgctttatttgatagaaagatcaaatctgcctttgtaaaacaagagactattttaatcattaagacaacacacatgtttgatttggaggcgtgttctcattcaaaaccttgcaaaatataatttcttcttgtctgggagatggtgtctttcttcggggagaaccttgagagaaatgtctatgaatgtgtctgtagattgaggataggccaacaaacatattttgaaatattttttctttcttttaaaaaaatagatacttttaaaagaaaatgcttatttaaattacatagctctttttttcactctcagtcatcatttgtggtttcagtcagtactacatttattccccagagctaggattgctgtgtttttagaaagggagcctgtttgaagggactctatactcttgcagtaattaaaatccttttaggagtctttgactttgacttactgctttgttttaaattattgaaattattcaacttccccattctaacccacggaaggctgttgttgttctcatttataatggcacaatataagtttaaagatagctgtctccacatgaatgcatctgcaaatccatctctcttctctcttctgccatccaggttaagaagctccactgcgctttgtccattgcaagttgagaagtgaggccccagctgtcacatacagctccatcacaagagggagccccattgctagtccccatcctcaaagcctctgcgtgatgttgtctactgttactcttcactaccctgtgggagccaaagccctggacataagaacagaaggcaagtaggtatacagctgcaattcagctctcttcactgtgtaatttttg

SEQ ID NO: 98 - RNA, 1428 nt, unidentified
  source 1..1428: mol_type = other RNA
caggcatgtgcctggctaattatttttattttttgtagaggtgagggtttagctttgttgctcaggctggtttcgaactcctggcttcaagcgatcctcccacctcagcctcccagtgttgggattacagatgtgagctactgtgcctggccaaaatatggtttaaaaatatgatttggtatggaatgattgcttattttttacataccccattggttctttctcctgtgcccaagctgaaaatcttccttctcttccactaacttcttacttagattttactttctgaataagaatcagtaaactttttctgtaaatggccagagggtaaatattttaggttctatgggctaaggggcaaaattgaagattttatgtagatacttgtagaaaaacagggaaaactaatttccacaacattttcactgatgaaattcaaaatataataatatcgagtacaaatttttgtaaatcaggtatgctaatcagaagaatgaaattctttttgtggggagatggagaataacatttttcttaattaggtttcaaagttcttattccctatcaaaatctattgcaaatgttcatctgtcaatcatgatctgtaatgaaattttacagattttatctttgaaaaatatattttcacacatgtaggcactgcccaatatgatgtcaatccaagggcatatgatttaaattaagtatattatgctcttggaaaacaattacagaaacatattattctcttcatatctgccttttagcatgtcagatgaatcatttctaactgatggttaggtggaagctcttcagttgcacagttaaatggattttgaaatatggaaatttcctttgtgtttgcagagatccgtaaaacaccactaaaactgtagtttgagcttgaaaaatataaccactgcaaacttgtgtgaggatagagagcttgctttttgttttaacttgtgactcaaataatgttagttgtcaaaatgacttcatcacagtaagttttgcatgtcagtactgttttgctttgtaatttatgttgactttattaggatgcatcatcaaatgatcaaaattagcaaactagcaaaaatgaatttcaaagtcattcagtcagtgatgtaaaataaataaaatcctgagccctctgccaactgcatgaacgtaccctcatggccaaggtgaccccagaaaaacctttaaaacagagttcctgatcataacaggacaggagggcagacatgcctcattaaactccctcccttttgaagtttagacacaacaaccgaccagcattgatgttaaagaagagatatcaagactgacagaaaggactttttgtggcaaaaaggtatcaaaagataaacaagacccaaggccatgccaggttatagataaggaagcaggaccggaagagtc

SEQ ID NO: 99 - RNA, 560 nt, unidentified
  source 1..560: mol_type = other RNA
ggtccacatatagcctcagttacattagctgcttatgaatgcaattcagttaattttcccgaaccaccctatcctgatcagattatttgtccagatgaagagggcactgaaggaaccatttctttgtggagtatcatctcaaaagttaggattgaagcctgcatgtggggtatcggtacagcaatcggagagctgcctccatatttcatggccagagcagctcgcctctcaggtgctgaaccagatgatgaagagtatcaggaatttgaagagatgctggaacatgcagagtctgcacaagactttgcctcccgggccaaactggcagttcaaaaactagtacagaaagttggattttttggaattttggcctgtgcttcaattccaaatcctttatttgatctggctggaataacgtgtggacactttctggtacctttttggaccttctttggtgcaaccctaattggaaaagcaataataaaaatgcatatccagaaaatttttgttataataacattcagcaagcacatagtggagcaaatggtggctttcattgg

SEQ ID NO: 100 - RNA, 619 nt, unidentified
  source 1..619: mol_type = other RNA
atgtattcggaaggaagtgacattgtacctcagagtaatgagacagctctccactactttaagaaagctgctgacatgggcaacccagttggacagagtgggcttggaatggcctacctctatgggagaggagttcaagttaattatgatctagcccttaagtatttccagaaagctgctgaacaaggctgggtggatgggcagctacagcttggttccatgtactataatggcattggagtcaagagagattataaacaggccttgaagtattttaatttagcttctcagggaggccatatcttggctttctataacctagctcagatgcatgccagtggcaccggcgtgatgcgatcatgtcacactgcagtggagttgtttaagaatgtatgtgaacgaggccgttggtctgaaaggcttatgactgcctataacagctataaagatggcgattacaatgctgcagtgatccagtacctcctcctggctgaacagggctatgaagtggcacaaagcaatgcagcctttattcttgatcagagagaagcaagcattgtaggtgagaatgaaacttatcccagagctttgctacattggaacagggccgcctctcaag

SEQ ID NO: 101 - RNA, 744 nt, unidentified
  source 1..744: mol_type = other RNA
gacttgattattcttagaaatgcacttgacctgcttttgccaaagcttgccagagtgatctctcggcttgccgactttgctaaggaacgagccagtctacccacattaggtttcacacatttccagcctgcacagctgaccacagttgggaaacgttgctgtctttggattcaggatctttgcatggatctccagaacttgaagcgtgtccgagatgacctgcgcttccggggagtaaagggtaccactggcactcaggccagtttcctgcagctctttgagggagatgaccataaggtagagcagcttgacaagatggtgacagaaaaggcaggatttaagagagctttcatcatcacagggcagacatatacacgaaaagtggatattgaagtactgtctgtgctggctagcttgggggcatcagtgcacaagatttgcaccgacatacgcctcctggcaaacctcaaggagatggaggaaccctttgaaaaacagcagattggctcaagtgcgatgccatataagcggaatcccatgcgttcagaacgttgctgcagtcttgcccgccacctgatgacccttgtcatggacccgctacagacagcatctgtccagtggtttgaacgcacactggatgatagtgccaaccgacggatctgtttggccgaggcatttcttaccgcagatactatattgaatacgctgcagaacatttctgaaggattggtcgtgtaccccaaa

SEQ ID NO: 102 - RNA, 439 nt, unidentified
  source 1..439: mol_type = other RNA
attatgtttgagaccttcaacaccccggccatgtacgtggccatccaggccgtgctgtccctctacgcctctgggcgcaccactggcattgtcatggactctggagacggggtcacccacacggtgcccatctacgagggctacgccctcccccacgccatcctgcgtctggacctggctggccgggacctgaccgactacctcatgaagatcctcactgagcgaggctacagcttcaccaccacggccgagcgggaaatcgtgcgcgacatcaaggagaagctgtgctacgtcgccctggacttcgagcaggagatggccaccgccgcatcctcctcttctctggagaagagctacgagctgcccgatggccaggtcatcaccattggcaatgagcggttccggtgtccggaggcgctgttccagccttccttcctgg

SEQ ID NO: 103 - RNA, 2992 nt, unidentified
  source 1..2992: mol_type = other RNA
agtttgaacaactgactcttgatggacacaaccttccttctctcgtctgtgtgataacaggcaaagggcctctgagggagtattatagccgcctcatccaccagaagcacttccagcacatccaggtctgcaccccctggctggaggccgaggactaccccctgcttctagggtcggcggacctgggtgtctgtctgcacacgtcctccagtggcctggacctgcccatgaaggtggtggacatgttcgggtgctgtttgcctgtgtgtgctgtgaacttcaagtgtttacatgagctggtgaaacatgaagaaaatggcctggtctttgaggactcagaggaactggcagctcagctgcagatgcttttctcaaactttcctgatcctgcgggcaagctaaaccagttccggaagaacctgcgggagtcgcagcagctccgatgggatgagagctgggtgcagactgtgctccctttggttatggacacataactcctgggccagaggctaaaaccccaggacccctgctgtccttcccgcagcttcttcttggagtctcagggcaaaccctttcgagcagcacctcccagtggccagaagctgaaatgacagcagtggtactgcctggtaaaagaattggttctgtgacccgggaagctttggttggccttgatttcttctctggaggcttggaaacgcttcctctcttcttctgttcttcacgccccatgcccctgctagcgtattactgttctgtgacttccctgtgacctctgcagaactcctcatcctgcgtttggtctccaggtgtcccctttctgccgtgttcctaacattttgattcctgtcttgaaaaaagcacctgctgcaccgtaagcccagggatgtggcagctgcagtgggcttggctttgtgaggaactgagtgtgtccacgttgggggaacatcatacttgatacacacgtttttatttgcacaaagaaaatgctatttttggagccagaattttcatgtctgatttatggtgattttcttaagaaccagaactgctggcagaaagggggcacccacacgcttagatagccgatgtcttattagagggcagtttgtggttcctgatttggaaattaacattctccaaacattccagtccaatgaaagttttatccgctttcccatataaaaattcttcccatgagagtgacttgattctcacaatcccgttggagtcgtgtgtgagtcctacagggtgagattcagcattgccatctccaagtgctcttcgtagggaaacagtttctgctcatgacgaggttccacttcccatctgatcccggcccggcctggaaacagagcacatgtgtttgaggatggcggtgtttggggacaggacatgagggtattgtgtggggctgctaggacaggcctggcggggtaggggggtgtccaagtcagtttacttggttcacaggttcccaggcccacccaggtgcctagaattggcctccaggatgggaccagaaagctggttttgcatagaaatggctagcagcaggcaccgtgccgctgtccactctctgcctgtgtctgccccagcacttggcacagcgggacagaagcagagatctgaatagtcaacccactcttcacaaagcttagaaagcggccgggcacagtggctcatgcctgtaatcccaacactttgggaggccaaggcgggcggatcacttgaggtcaggagttcgagaccagcctggccaacatggtgaaatcccatctctacaaaaatacagaaattagccaggcatgatggcgagtgcctgtaatcccagctacttgggaggctgaggtgggagaattgcttgaacccaggaggtggaggttgcagtgagctgagattgtgccactgcactccagcctgagtgacagggtgagactaagtctcaaaaaaaaaaaaaaaaaaccacacgcaccacacacacacccacacacacacacagcttagaaggggctgctgttctcataagcacagatgtctgaagagccgttagccagaatgattctttttttttgttttgagatatgatcttgttctgtcacccaggctggagtgcagtggcacagtcattgctcactacagcctcgactcctgggctctagcaatcctcccacttcctgagtagctgggatgacaggtgcgtgccaccatgccagtaatttttttattttgtagagatggggtcctgaacgcatggcctcaagtgatgctcctgcctcagcctcttttattatttgtttttagacgaagttttactctgttccccaggctggagtgcagtggcacaatctcagctcactgcaacgcctcccaggttcaagtgattctcctgcctcagtctcccgagtagctttgactataggcgtgcaccaccacgcctggctaatttttgtgtttttagtagagatggggtttcaccatgtgggccaggctggtcttgaactcgacctcccgtgatctgctcacctcagcctcccaaagcctcagcctcttacagtgttgggattacaggcgtgagacactgtgacccgggacgattttcaatcacagttttttgttacgagtggaaaatgcgtatttataagaatgaagtagtacagacatgaatgtgtagaaatctctataatcctgccatccaaggatggcacctgttaatgtgtatatcagggatgtccaatcttttggcctccctgtgccacactggaagaagaagaatcgccttgggccacgcataaaatacactaacactagcaatacctgatgagctaaaagaaaaaaaatcacaaaaaaacctcgtactgtttgaagaaagtttacagatttgcgttgggccgcaagttggacaagactgctatatgtatattctaggttttcccctataggtatacttatgtgaaaatgattattgtgataaattttgttttgagatgaa

SEQ ID NO: 104 - RNA, 171 nt, unidentified
  source 1..171: mol_type = other RNA
agtttgaacaactgactcttgatggacacaaccttccttctctcgtctgtgtgataacaggcaaagggcctctgagggagtattatagccgcctcatccaccagaagcacttccagcacatccaggtctgcaccccctggctggaggccgaggactaccccctgcttctag

SEQ ID NO: 105 - RNA, 2156 nt, unidentified
  source 1..2156: mol_type = other RNA
gcagttcctctttgtggttgccttcactaccttcctggtcagctgcgtggactatgacatcctatttgccaacaagatggtgaaccacagtcttcaccctactgaacccgtcaaggtcactctgccagacgcctttttgcctgctcaagtctgtagtgccaggattcaggaaaatggctcccttatcaccatcctggtcattgctggtgtcttctggatccaccggcttatcaagttcatctataacatttgctgctactgggagatccactccttctacctgcacgctctgcgcatccctatgtctgcccttccgtattgcacgtggcaagaagtgcaggcccggatcgtgcagacgcagaaggagcaccagatctgcatccacaaacgtgagctgacagaactggacatctaccaccgcatcctccgtttccagaactacatggtggcactggttaacaaatccctcctgcctctgcgcttccgcctgcctggcctcggggaagctgtcttcttcacccgtggtctcaagtacaactttgagctgatcctcttctggggacctggctctctgtttctcaatgaatggagcctcaaggccgagtacaaacgtggggggcaacggctagagctggcccagcgcctcagcaaccgcatcctgtggattggcatcgctaacttcctgctgtgccccctcatcctcatatggcaaatcctctatgccttcttcagctatgctgaggtgctgaagcgggagccgggggccctgggagcacgctgctggtcactctatggccgctgctacctccgccacttcaacgagctggagcacgagctgcagtcccgcctcaaccgtggctacaagcccgcctccaagtacatgaattgcttcttgtcacctcttttgacactgctggccaagaatggagccttcttcgctggctccatcctggctgtgcttattgccctcaccatttatgacgaagatgtgttggctgtggaacatgtgctgaccaccgtcacactcctgggggtcaccgtgaccgtgtgcaggtcctttatcccggaccagcacatggtgttctgccctgagcagctgctccgcgtgatcctcgctcacatccactacatgcctgaccactggcagggtaatgcccaccgctcgcagacccgggacgagtttgcccagctcttccagtacaaggcagtgttcattttggaagagttgctgagccccattgtcacacccctcatcctcatcttctgcctgcgcccacgggccctggagattatagacttcttccgaaacttcaccgtggaggtcgttggtgtgggagatacctgctcctttgctcagatggatgttcgccagcatggtcatccccagtggctatctgctgggcagacagaggcctcagtgtaccagcaagctgaggatggaaagacagagttgtcactcatgcactttgccatcaccaaccctggctggcagccaccacgtgagagcacagccttcctaggcttcctcaaggagcaggttcagcgggatggagcagctgctagcctcgcccaagggggtctgctccctgaaaatgccctctttacgtctatccagtccttacaatctgagtctgagcccctgagccttatcgcaaatgtggtagctggctcatcctgccggggccctccactgcccagagacctgcagggctccaggcacagggctgaagtcgcctctgccctgcgctccttctccccgctgcaacccgggcaggcgcccacaggccgggctcacagcaccatgacaggctctggggtggatgccaggacagccagctccgggagcagcgtgtgggaaggacagctgcagagcctggtgctgtcagaatatgcatccacagagatgagcctgcatgccctctatatgcaccagctccacaagcagcaggcccaggctgaacctgagcggcatgtatggcaccgccgggagagtgatgagagtggagaaagcgcccctgatgaagggggagagggcgcccgggccccccagtctatccctcgctctgctagctatccctgtgcagcaccccggcctggagctcctgagaccactgccctgcatgggggcttccagaggcgctacggtggcatcacag

SEQ ID NO: 106 - RNA, 138 nt, unidentified
  source 1..138: mol_type = other RNA
gatgctgatcctcccgctggcttccagacagacctgggacttggcagtcatgccgggtgatggtgttcctgcggagaccctcagttgtcctattccttcctagcttccctgcaataaaatcaagctgcttttgttgga

SEQ ID NO: 107 - RNA, 1354 nt, unidentified
  source 1..1354: mol_type = other RNA
gtggcccgtgatgtagccgtggtggccggagggctaggtcgccagctgctacaaaaacagccagtatcacctgtgatccatcctcctgtgagttacaatgacaccgctccccggatcctgttctgggcccaaaacttctctgtggcgtacaaggaccagtgggaggacctgactcccctcacctttggggtgcaggaactcaacctgactggctccttctggaatgactcctttgccaggctctcactgacctatgaacgactctttggtaccacagtgacattcaagttcattctggccaaccgcctctacccagtgtctgcccggcactggtttaccatggagcgcctcgaagtccacagcaatggctccgtcgcctacttcaatgcttcccaggtcacagggcccagcatctactccttccactgcgagtatgtcagcagcctgagcaagaagggtagtctcctcgtggcccgcacgcagccctctccctggcagatgatgcttcaggacttccagatccaggctttcaacgtaatgggggagcagttctcctacgccagcgactgtgccagcttcttctcccccggcatctggatggggctgctcacctccctgttcatgctcttcatcttcacctatggcctgcacatgatcctcagcctcaagaccatggatcgctttgatgaccacaagggccccactatttctttgacccagattgtgtgaccctgtgccagtgggggggttgagggtgggacggtgtccgtgttgttgctttcccaccctgcagcgcactggactgaagagcttccctcttcctactgcagcatgaactgcaagctcccctcagcccatcttgctccctcttcagcccgctgaggagctttcttgggctgcccccatctctcccaacaaggtgtacatattctgcgtagatgctagaccaaccagcttcccagggttcgtcgctgtgaggcgtaagggacatgaattctagggtctcctttctccttatttattcttgtggctacatcatccctggctgtggatagtgcttttgtgtagcaaatgctccctccttaaggttatagggctccctgagtttgggagtgtggaagtactacttaactgtctgtcctgcttggctgtcgttatcgttttctggtgatgttgtgctaacaataagaagtacacgggtttatttctgtggcctgagaaggaagggacctccacgacaggtgggctgggtgcgatcgccggctgtttggcatgttcccaccgggagtgccgggcaggagcatggggtgcttggttgtttccttcctaataaaataaacgcgggtcgccatgc

SEQ ID NO: 108 - RNA, 1248 nt, unidentified
  source 1..1248: mol_type = other RNA
gaggaaagtgctcagagacaaaaaacagctggcaaaatcttcagagccgaggcgtccactgggcaggatgccccgaggcaggcccaggcgaggagctgggcatcacagggaaaggccgcctctgcagagggggatgagagcgaggtctcggagcatctcagtgccagctcggcttctgccatccagcaggacagcacttccagcatgcagccaccatctgaagcccccatggtgaacacagtcagctcagcttattcggaggattttgaaaactctccaagtctgacagcatctgagccaaccgcccattccaaggagtctcttgacagaacactggacgctttgtctgaatcctcttcaagtgtgaagacagaccttccacaaacagccgagtctaggaaaaagtcgggcaggcacgtgacaagagtgcttgtgaaggacacagctgtgcagacgccagatcctgccttcacctacgagtggaccaagggtaagccttggggcccgtgggggccggggcagccgggacaggagctcagcgtgtggtttacagcatcctgggagacacgtgggagagctcaggttcctgagatttgggctcttgttattaatcacctggaggggttggccaagcaaggtggctcacgcctgtaatcccagcactttgggaggccgaggcgggaggatcacttgaggtcaggagttcaagaccagtctggccaacacagggaaccctcatctctactaaaaatacaaaaattagctggctgtggtggtgtgttcctgtaatcccagctactcaggaagctgaggcttgagaagaacccgggaggtggaggttgcagtgagctgagatgatgcccctgcactccagcctgggtgacagaatgagactctgtctcaaaaaaaataaaagtcacctagggaccttggctcaccatttcctcttccatggacgagggacagccgccctcgtgtgtcccgatgtcttcctagccctgaagcattcagaaggatcagaaccaaggactagggtagcacctcccacaggatgattcacggggggactccagcaaggtctggagacattattagttgtcacaactcagggtggggaatgctatcggcatctgctgggtggaggccaggggtgctattccacaccccgcagtgcacaggatgcctttgtcctggagaatgaacaccctgggtgactttccagatatttctctctctctctctcactct

SEQ ID NO: 109 - RNA, 3728 nt, unidentified
  source 1..3728: mol_type = other RNA
cttcctttccttacgcacgctgcgcgcggacgtcggcctctgacgtcgtcgcctcagcgccggctcccggccgggccgcggccgccgaccgttgagccgccggctgagccgcctgctgaagtccctccctcaggaacccctccgccaccctccacctccgaaccgctctcgcggcggcgacccatgtgggggttcaggctcctgcggtcgccgccgttgctgctcctgctgccgcagctcggaatcggaaacgcctcgtcctgctctcaggccagaaccatgaacccgggcggcagcggcggcgcgcgatgctccctctcggccgaggtgcgccgccgtcagtgcctgcagctttccaccgtgcctggagccgatccgcagcgcagcaacgaattgctcctgttggcggcggccggggagggactggagcggcaggacctccccggggacccagcgaaggaggagccgcagccgccgccccagcatcacgtcctctatttccctggggatgtgcagaattaccatgaaattatgactcgtcatcctgagaattatcaatgggaaaactggagtctagaaaatgttgctaccattttagcccaccggttccccaatagttatatttgggtgataaaatgttcccgaatgcatttgcacaaattcagctgctatgacaattttgtgaaaagtaacatgtttggtgccccagaacacaatactgactttggagcttttaagcacctttatatgttattagttaatgcttttaatttaagtcagaatagtttatcaaagaaaagtttgaatgtttggaataaggactccatagcatctaactgtagatccagtccttctcatactacgaatggttgccagggagaaaaagtgaggacctgtgaaaaatctgatgagtctgccatgagtttttatccaccatcactaaatgacgcatcttttactttgattggattcagtaaaggttgtgttgttttgaatcagttgctttttgaattgaaagaagccaagaaagacaagaacatagatgcttttatcaaaagcataagaacaatgtattggctggatggtggtcattctggaggaagcaatacttgggttacttatccagaagtcttgaaagaatttgcacaaacaggaattatcgttcacactcatgtaacaccttaccaagtacgtgatccaatgagatcttggattggaaaggagcacaagaaatttgttcagatacttggggatcttggtatgcaggtgactagccaaattcattttacaaaggaagctccttccatagagaatcacttcagggttcatgaagtattttgagattacaggtatattaatgaacttgttcagtggaagaacataagcacttttgagtgttataaattcagataatgggatgtaattcatagctgcattgtcagttttggggtatggggggaagcacacattcctaaaatgtgagtgtaatgtgcaatagtattttttgcttgtgaatgtgagcagttattaatttggattgagttagaattagttaatttgaaatctaacaaggtggtttgtaataatgctgaggagatataagacccttaaaatgaaagttacaacattgttcttataaaaggtaactaaaattgttactgttggaaataactgattttctgagtaatgttttaaactaatttggtgacattttaacagtaattagctattttgagtggaaatattttcatttctcttcaaacaaaagcaaaggtacgatgctgttttctatcattttggaataactgcaccctgccttttgtgtttttgtaaactccttgactcattctttcatgtgtcaccaagtacttttctcatgagagtcaacatatatttgtttccaaatgtccacaagtgtacaatagtgtaaaggtggtttttaaaaacatagccaggtgtggtggcacgtgcctttagttccagctactcaggaggctaaggcaggaggattgcttgagcccaggctgtgtggttcaccataattgtgtttgtgactagctactgcactccaacctgggcaacatagtgggacttcatctctaaaacaaaacaaaacaaaattacacttaagcactattgtttaatttttaattgtcagtttatcattattttgggtaagacattctggggtttcttgaatcttgtccaaaaaccagttgttttggaaaattgctttaaattgagcatatttatgtatattggataaaaatgtactacagagcaaatttcaaatttttcattatatcagtctttttgaaaggatcaacttggataaaataaatatataatgctctatttgttagagctctattaaaaaggaaacagattccatagatctaagtcaatgtttctccagaagcatgattttgtctgccaaaagaaaatagctctctttggccaaaatgcaaaattacattgctataagaaaagttacaagggaaagtttgaagacacaaatgatttaattttggctcaaaaactgaatttgcttaacactgctacataatttgggtgaagtttccttctgcccgtttttcttgacctagataaatacactttgagaaatccagatctaataaatgtcaaccaacattgacattgtaattgggtgattacaataaaaggtgagcagtttgttgtttattaataattagcttttgcaggtaatgaaatagcagggaagtaacatgctgctttaggactaaaaaaaaaaaaaaaaaaaagacactgaagcttaataccttaatgacccagaagacctagatttataaagccatatagaataaaaatgttaatttcttggtttcttcccagaattatattttaatgactccataaatacattccaaatattcagagattcatgagaaaggattcttaacaaatgtttaaaaataacatttctttttggtgttttaacacttaattttataaactgcagaacagcatctctaaatggctttttttttttttttaaagacaaagtagtattatgttgcttcagtttctttaaatacctcgtttcttttgagaccaaaaattcccatcaaggaaataaatcagcctgattttaacattttacatattacattgcctttaagcactatgattggatggctttttttctatttgtttaataatccttggctaaattcacatgtatctaaaggaaaatagctgagttactgaattctataaggggaagaaaaagcatttattttcacatgattaactgaaatggaaaagtaagacatttagcagaataattcatttaaaaataattttaaaaaatcagacatatttaaaaatctaggttgtctatccagtatgtgaatgcttaattataattgttacatgttgaatgggtattaagagtaagtcaccaggtacacaaaactgccatcatagcaaaatgagacagtgttgaaaagacaaaatattttctggaatgaaaatatattcaagttgatccatattcagatgttttctgtttaatacttcagattggtcctttgtccacatttgtttaaaacttattgtaatgttaatttacacttttggattgtgcctttgtcatgagttgagaaaagtagtcctacaaataatgtgaaagttgttaccaccacagcatgaatgtataattgtattaaaattgatcatccacagaa

SEQ ID NO: 110 - RNA, 394 nt, unidentified
  source 1..394: mol_type = other RNA
gacctcagcaatggagaatatccccatcatcacaactgtcatcactctgccgcacgtgattgtggagaatatccctctccatgtgaatgcagatctgccgtcatttgggcgtgtcagagagtcgttacctgtcaagtatcacctacagaataagaccgacttagttcaagatgtagaaatttctgtggagcccagtgatgccttcatgttctcaggtctcaaacagattcgattacgtatcctccctggcacggagcaggaaatgctatataatttctatcctctgatggctggataccagcagctgccatctctcaacatcaacttgcttagatttcctaacttcacaaatcagctgctcaggcgttttatacctaccagtatttttgtcaag

SEQ ID NO: 111 - RNA, 4539 nt, unidentified
  source 1..4539: mol_type = other RNA
attcgaatgaaaggatcccttccttcactgagtccttttcctcagtcattacttaattactgtaaaggaggtatcgcattttttagacctggagcagctggagaccacaagcttgatgaagtttccattagagcaataggttgcttcagagaactttgtgctctgtgttggatgctgcatgtccgtgataagttatcctatagttgcaggcaatatcagaaagcaagagaaaatgtaaaaggagaaaaggaccttgaagtggagtttgattcttgtatgattgagcactgtcttagtgcagtggaatgggcttatagaatgctgcctttctctcggttttttaatatggaagaacttattcaggatataattttgagccttattggagaactgccaccaatcagaaaggtagcagaaattttcgtgaaagcatttccctatcctgaggacgtgagggttcctttaagagacaaatatcactctcttcaccagagactcagacactgtgttgtgaaaggaccccagactgaggaaatgatgtctgttgtcatgcattctatccagaaagtgagggtgaaagctctaaaacgtgtgcagagaaatataggctcttttgaagtgaatatatgggaaccaattgaagaagagaaaccagatgaggctccaggtgttgacagatattccctggggactagtttgagcagaagtacactcacagaactaggagattctgtggttcacagtgatgcagatacgttctctgaagctttgtcggttgaagaaaaaagtaggataaatatctatcaaagaaatgccccaaatcacatggaattaacatcaattcataagccaactgataaaaggaaaatgtgtaatcagaaagaaaatcctacaaagaaagaagatcatgaaaagttatcacaaaatacacttcctgtaataggtgtttgggaatttgaacgtgatgatgatgaatatattaaattccttgatctgtttttgagttacattcttgaaagagacctaccttattccagggatgctgacattccatttctaactagtttttctggaaagcttagagaacatgaacttaattctttactttttgatgtacatacaacattaaaacgacatcagagcaaaactaaaagccagaatgtgtttagagctggttcttgctttgttgttgctcctgagtcctatgaatcagaaaaatcatcctctttaaatgatgaatatggcatgcatttagaaaaccagaaactttcatcatcagtactggttaatcaagggatcaaaccttttttacaatatccttcgaatgaagtcaataagaatgaaggaatgagtggattatttggtttaaaacaaaggtcaatttacaaaatacaagatgacactagagagaaatgtctaatccagagatcatcaaaccacattttttggactcccaagtccattaaaactagaagatgtattttcaaagctattcagtgcaatgatattaaccctcaagaagatcttcctttagcactaaacacttttggcagtataggaagactgctggaatggatgataaggtggtctaatagaaggctactctgtgattctggtataactgagtcatcctctgagtacagtccagtaattcgtgtaaagacctctacagctgccattcttacatcattatggcttttggaacaaccctattttgctacatataaggcaaaaaatgccattattaagatggtagagaatcgtgacactgggtgtcagattggacccaatattgagagggagagcaaatcagatgctggcggttcagttgcagtagcaactccaggtggaactgaggaaagaaatggtcagaataaatcttgtcaaaatatcttgaatagaatgccaactgaagcaaaaaatcctgatataaaagaaatcaatgatgatattatttccatcactcataatactaaaaaagaatttatagatattgatgagaatcttttagaagtagaagcatttacagaagaggaaatggatatgcacatatcagactatgaagaagacattgaagaatctgttggaggtttcagaagtcccagtcttgccatttgcatgatgactttaccacagcagttagaagaagagttcacagaagaggttcagtgtcaaagggaagaaccactggagacaattatggaggaaaaatcgactgaacaaaaaggtatgatcgaagccttttcacatcctgggcataccactcctcaatcaatgcaagtagatacgagttcagaaatttctagtgcacagatttctacatataaagaaaaatcttcctcagttccacttctgatatcaaatggagtcaatgttgcttcacaaccacctgctccaacacctcagaagacccagagaaatgaattcacggctcagttaccagattgttcggagtccgttaggcagatgctgcaagatgaaatgtttaaattagttcagctgcaacagatcaacttcatgagcctaatgcaaatagtaggatcatcctttgctaatctcccagatacacaacaacttgtacagcagtctcagtctgtgcatttaggggaaagccaagaatcaaacctaagaggatgtggtgatgttgaagacagcaacaaaaatcttaaggagagattttttattaaaccacagtcaatgggagagaacgccagagagcctcgcaagaacagcccacactgccatgaaggaactatcccatctggtcaaaatagtactggaaacgtacagaatgttccacatgggagtattcctttatgtcaattaaatggccagccccggaaaaaaggaccaattccatcatctcaaaacttaccatccacttcgttttatccagctcctgctggaaatactcacctctaccttttgtccacaccttctgttgttcagaaggcacctagacttatcccacatgcaaaaacatttagtcctggtgatggctttcctttgcttcaatttaagtctaaacaagaattccagccccttttcttacatacaggaagtattccacaagttcccttcaggcctttgccacaaccaagagaggcttggggattatctgactccttccaacctgctctgccacagagagcagcacaaactactccagcatcccatttgaatgtaagccagtataacactgaagccagaaaaaaagaagttgagcagaagacgtgggcagaaactgtaattacagaaattcctaatcatgtgaacttggatcaatatgttggacaagaaaatttgacacctcaacaggactcttcagtgtttataaaaccagaaaaactatttgatgttaagccagggacccttgagatatctcctcaccattcctttggacttccgttactatacctgccacttaaacctcctaatatgtttccatcaacctcaagagcatctattacagttccctcaacacctatccaacctatagcagaagaaagaaaatacccaagattgtcattacttcattcacatttgtccccagaaaataggtgcaaaaaaacacaacttatcccacttgaaaacctcattgcgtttaaacaaagccaacagaaactaacacataatttatttgaacaaggtgatgctggacaccttcaacttctaaaggtcaaaatagaaccacctgaagtaagacaaggaaaggacagtaaaaaaaggcaaagaagaagagctgagaaagagctgcaagaaaaaagatgtgagaaactgaggagaaaaccaaatgtgacttttcgaccagagaattccataattaataatgatgattcagaaatcattaagaaacccaaggaacaacaagaacattgtggttcccatcctttggatgacttcgacgttccttttgaaatgctacaagatgataatacttcagctggattgcatttcatggcctctgtaaaaaagaaagctataggaagtcaagatgcaagtacaaatacagacccagaacatgagcctttgactgctcctcagctcttggtcccagatgtctatctaaatctgaagctttccagtgaaatgtcagagaaaccttggtcaccctcaatacctcatacagtaacaaacttggaattacctgtgagagaagagccttcaaatgataatgttatcaaacagcaaagcgatcatctagcagttccatcgtctgcagagttacattatatggcagcttcagttactaatgctgttcccccacataattttaagagtcaaggtctgccaaaaccagagttccgattcaaaggacagagcacaaagtcagactctgcagaagattatctattgtggaaacggctgcaaggtgtctctgcagcttgccctgcaccaagctctgcagctcaccaactagagcatctcagtgctaagcttcagaaaattgacgagcagttgctagcaatacagaacattgctgaaaacatagaacaggatttccccaagcctgaaatgctagatctacattgtgataag

SEQ ID NO: 112 - RNA, 1176 nt, unidentified
  source 1..1176: mol_type = other RNA
agggaccacagagcaccagcctgcatggaacttccttcctcactcagcttcccacgttgccagctgggacaggggagatggagtaattttgctgtggaaagacttcacgtcttgccgaatgaaagtaagcaaagatactgctagagtcgggaatctagagtcgggaattcattaagccaagggagtaagaatggccacagagtagtgaaatactgtttcttctggaaaagtcagcctccagcgcctgcagcttccggaactaagatgtgactgggcttgcggaggcgctgactcctctcctccctccctggctgtgcaggtcccgcctgtctgtcacgctgatgcccgtgcagctgtctgagcacccggaatggaatgagtctatgcactccctccggatcagtgtggggggccttcctgtgctggcgtccatgaccaaggccgcggacccccgcttccgcccccgctggaaggtgatcctgacgttctttgtgggtgctgccatcctctggctgctctgctcccaccgcccggcccccggcaggccccccacccacaatgcacacaactggaggctcggccaggcgcccgccaactggtacaatgacacctaccccctgtctcccccacaaaggacaccggctgggattcggtatcgaatcgcagttatcgcagacctggacacagagtcaagggcccaagaggaaaacacctggttcagttacctgaaaaagggctacctgaccctgtcagacagtggggacaaggtggccgtggaatgggacaaagaccatggggtcctggagtcccacctggcggagaaggggagaggcatggagctatccgacctgattgttttcaatgggaaactctactccgtggatgaccggacgggggtcgtctaccagatcgaaggcagcaaagccgtgccctgggtgattctgtccgacggcgacggcaccgtggagaaaggcttcaaggccgaatggctggcagtgaaggacgagcgtctgtacgtgggcggcctgggcaaggagtggacgaccactacgggtgatgtggtgaacgagaacccggagtgggtgaaggtggtgggctacaagggcagcgtggaccacgagaactgggtgtccaactacaacgccctgcgggctgctgccggcatccagccgccag

SEQ ID NO: 113 - RNA, 420 nt, unidentified
  source 1..420: mol_type = other RNA
aaaggcaggcattctctttccttttccttctctcctgagcgctcctgcagttcctggggcgtagtaggggatccacaagcgtttgtgaccagtgaagttctttacaagggtgagatctgcacgggaggacccgagcgagggtctcggcttgccaggaagccggggttccccgggaagcgtggagttcacccgcgcactcgaagtgcctttgcaaaattatatctgggtgttggcacccagccactattctgccaatgaagtacatcctggtcacgggtggggtcatctcaggcattggtaaagggatcattgccagcagcattggaacgattctaaaatcatgtggactccgagttactgccataaaaatcgacccctatattaacatcgatgctggcactttttcaccttatgaacacg

SEQ ID NO: 114 - RNA, 633 nt, unidentified
  source 1..633: mol_type = other RNA
ggattttgcttccgggtcgtaggcgctagctctgggcgcagaggtttctgggagccaagagtggtaatggcgtctgtatgatcttcggagcctgctgcatcggacctcggccagtcataaaagatgacaacagcagccaggccaacctttgaacctgccagaggtggaaggggaaaaggagaaggtgatttgagccaactttcaaagcagtattcaagcagagacctaccctctcatacaaagataaaatacagacagactactcaggatgcccctgaagaggttcgtaaccgtgacttcaggagagagttggaagaaagagagagagctgctgcaagagagaaaaatagggatcgtccaacccgagaacatacaacctcctcttcagtgtcaaaaaagccacggttagaccagattcctgccgccaaccttgatgcagatgaccctctaacagatgaggaagatgaagattttgaagaagaaagtgatgatgatgatactgcagctcttcttgcagaactggaaaaaattaaaaaagaaagagctgaagagcaggccaggaaggaacaagaacaaaaagctgaagaagagaggattcgtatggaaaacattctgagcggaaaccctctcctt

SEQ ID NO: 115 - RNA, 584 nt, unidentified
  source 1..584: mol_type = other RNA
atttcctccttcttcacccctgaaaccatatccaaagaagcccccaaaccaaaaggatgttattaaatttccagaatggaatgatctcccaccaggcacttcacaagagaacatcccagtgaggccagttgtgatgaattctgagatgtggtacaagcgtcacagtattgcaattggagaggtgccagcttgccgtcttgtccaccgcagacagctgacagaggccaatgtagaagagatatggaagtctatgacattatcatacttacagaaaattcttggcctggattccttagaagaagttttagacgtcaaacttgtcaattcgaagttcatcatccataatgtatatagtgttagcaagcagggagttgttattcttgatgacaagtcaaaagaacttcctcattgggtgctgtcagctatgaagtgtttggcaaattggcccaactgttctgatttgaagcagcctatgtacttgggatttgaaaaagatgtctttaaaaccatagctgattactatggtcacttgaaagagcctctacttacatttcatctttttgatgcttttgtcagtgtactgg

SEQ ID NO: 116 - RNA, 2633 nt, unidentified
  source 1..2633: mol_type = other RNA
cttctctctcggcgtttccgctgtcagggccctgcggtgtgactcgcgggctcagctggttggtattttttgggcctcgtgcttcgtggtgggagacccaggtcgaggtccggccgtagcacctccgcgccgtcgccatgtcgcggtttttcaccaccggttcggacagcgagtccgagtcgtccttgtccggggaggagctcgtcaccaaacctgtcggaggcaactatggcaaacagccattgttgctgagcgaggatgaagaagataccaagagagttgtccgcagtgccaaggacaagaggtttgaggagctgaccaaccttatccggaccatccgtaatgccatgaagattcgtgatgtcaccaagtgcctggaagagtttgagctcctgggaaaagcatatgggaaggccaaaagcattgtggacaaagaaggtgtcccccggttctatatccgcatcctggctgacctagaggactatcttaatgagctttgggaagataaggaagggaagaagaagatgaacaagaacaatgccaaggctctgagcaccttgcgtcagaagatccgaaaatacaaccgtgatttcgagtcccatatcacaagctacaagcagaaccccgagcagtctgcggatgaagatgctgagaaaaatgaggaggattcagaaggctcttcagatgaggatgaggatgaggacggagtcagtgctgcaactttcttgaagaagaaatcagaagctccttctggggagagtcgcaagttcctcaaaaagatggatgatgaagatgaggactcagaagattccgaagatgatgaagactgggacacaggttccacatcttccgattccgactcagaggaggaagaagggaaacaaaccgcgctggcctcaagatttcttaaaaaggcacccaccacagatgaggacaagaaggcagccgagaagaaacgggaggacaaagctaagaagaagcacgacaggaaatccaagcgcctggatgaggaggaggaggacaatgaaggcggggagtgggaaagggtccggggcggagtgccgttggttaaggagaagccaaaaatgtttgccaagggaactgagatcacccatgctgttgttatcaagaaactgaatgagatcctacaggcacgaggcaagaagggaactgatcgtgctgcccagattgagctgctgcaactgctggttcagattgcagcggaaaacaacctgggagagggcgtcattgtcaagatcaagttcaatatcatcgcctctctctatgactacaaccccaacctggcaacctacatgaagccagagatgtgggggaagtgcctggactgcatcaatgagctgatggatatcctgtttgcaaatcccaacatttttgttggagagaatattctggaagagagtgagaacctgcacaacgctgaccagccactgcgtgtccgtggctgcatcctaactctggtggaacgaatggatgaagaatttaccaaaataatgcaaaatactgaccctcactcccaagagtacgtggagcacttgaaggatgaggcccaggtgtgtgccatcatcgagcgtgtgcagcgctacctggaggagaagggcactaccgaggaggtctgccgcatctacctgctgcgcatcctgcacacctactacaagtttgattacaaggcccatcagcgacagctgaccccgcctgagggctcctcaaagtctgagcaagaccaggcagaaaatgagggcgaggactcggctgtgttgatggagagactgtgcaagtacatctacgccaaggaccgcacagaccggatccgcacatgtgccatcctctgccacatctaccaccatgctctgcactcgcgctggtaccaggcccgcgacctcatgctcatgagccacttgcaggacaacattcagcatgcagacccgccagtgcagatcctttacaaccgcaccatggtgcagctgggcatctgtgccttccgccaaggcctgaccaaggacgcacacaacgccctgctggacatccagtcgagtggccgagccaaggagcttctgggccagggcctgctgctgcgcagcctgcaggagcgcaaccaggagcaggagaaggtggagcggcgccgtcaggtccccttccacctgcacatcaacctggagctgctggagtgtgtctacctggtgtctgccatgctcctggagatcccctacatggccgcccatgagagcgatgcccgccgacgcatgatcagcaagcagttccaccaccagctgcgcgtgggcgagcgacagcccctgctgggtccccctgagtccatgcgggaacatgtggtcgctgcctccaaggccatgaagatgggtgactggaagacctgtcacagttttatcatcaatgagaagatgaatgggaaagtgtgggaccttttccccgaggctgacaaagtccgcaccatgctggttaggaagatccaggaagagtcactgaggacctacctcttcacctacagcagtgtctatgactccatcagcatggagacgctgtcagacatgtttgagctggatctgcccactgtgcactccatcatcagcaaaatgatcattaatgaggagctgatg

SEQ ID NO: 117 - RNA, 1342 nt, unidentified
  source 1..1342: mol_type = other RNA
gattatttttctcctcttccttttttaatctatcctcttttcaccacattttagtgagaaagcaattttttttttacaaatggagcttatattgatttttatttcataaataattgaaagagggtagaaaaatctgcccttcatttaaaaattgaatgtttcactatgtatttaaaaagcgattcaggattaggaggatggataaccataccagctgtagctgatgttctaaaatattcttgcattgtttgctggtcttctagggaaaagaataatgtggaacaggaccttaaggagaaggaagatactattaaacagaggacaagtgaggttcaggatcttcaagatgaagttcaaagggagaatactaatctgcaaaaactacaggcccagaaacagcaggtacaggaactccttgatgaactggatgagcagaaagcccagctggaggagcaactcaaggaagtcagaaagaaatgtgctgaggaggcccaactgatctcttctctgaaagctgaattaactagtcaggaatcgcagatctccacttacgaagaagaattggcaaaagctagagaagagctgagccgtctacagcaagaaacagcagaattggaggagagtgtagagtcagggaaggctcagttggaacctcttcagcagcacctacaagattcacaacaggaaattagttcaatgcaaatgaaactgatggaaatgaaagatttggaaaatcataatagtcagttaaattggtgcagtagcccacacagcattcttgtaaacggagctacagattattgcagcctcagcaccagcagcagtgaaacagccaaccttaatgaacatgttgaaggccagagcaacctagagtctgagcccatacaccaggaatctccagcaagaagtagtcctgaactactgccttctggtgtgactgatgaaaatgaggtgactacagctgttactgaaaaagtttgttctgaactcgacaataatagacattcaaaagaggaagatccatttaatgtagactcaagttcgctgacaggtccagttgcagatacaaacttggattttttccagtctgatccttttgttggcagtgatcctttcaaggatgatccttttggaaaaatcgatccatttggtggtgatcctttcaaaggttcagatccatttgcatcagactgtttcttcaggcaatctactgatccttttgccacttcaagcactgaccctttcagtgcagccaacaatagcagtattacatcggtagaaacgttgaagcacaatgatccttttgctcctggtggaacagttgttgcagcaagcgattcag

SEQ ID NO: 118 - RNA, 7342 nt, unidentified
  source 1..7342: mol_type = other RNA
gtggcgacagacaaagttgcagaaaagctgagctctactctctcatgggtgaagaacacagtatcgcatacagtcagtcagatggccagtcaggtggcaagtccatctacttcattacataccacatcctcatctaccacactatcaacaccagccctttcaccatcttccccatcacagttgagtccagacgacttagaactcctggctaaactggaagaacagaatagattgttagaaacggatagtaagtctttaagatctgtaaatgggtcaagaagaaacagtggctcttctcttgtgtcgagttcatcagcctctagcaacctcagtcaccttgaagaagattcttggattctttggggaagaattgttaatgaatgggaagatgtacgcaaaaagaaggaaaagcaagttaaggaacttgttcataaagggataccccatcactttagagcaatagtttggcaacttttatgcagtgcacaaagtatgccaattaaggatcagtattcagaactcctgaaaatgacctcgccttgtgaaaaattgatccgaagggacattgctagaacttaccctgaacacaacttttttaaggaaaaagatagccttggacaggaggttttatttaatgtaatgaaggcttactctttagtagatcgtgaggttggttactgtcaaggaagtgcttttatagttggattgttgcttatgcagatgccagaagaagaagctttctgtgtatttgttaaattaatgcaagattatagacttcgtgaactttttaaaccaagtatggcagaattgggcctttgtatgtaccagtttgaatgtatgatacaggagcatcttccagagctctttgtacattttcaatctcagagttttcatacctcaatgtatgcatcatcctggtttctgactatctttcttacaacttttccactaccaattgcaacaaggatatttgatatctttatgtctgagggtttagaaatagtgtttcgtgtaggattagcacttcttcagatgaatcaggcagaactgatgcaacttgacatggaagggatgttacagcactttcaaaaggtcattccacatcagtttgatggtgtcccagacaagctaatccaagcagcttaccaagtcaaatacaattcaaaaaaaatgaaaaagcttgaaaaggaatacactacaataaaaacgaaagaaatggaagagcaagttgaaattaaaaggttacgcacagaaaatagacttttaaaacagcgcatcgagacattagaaaaacataaatgcagttccaactacaacgaagattttgtgctacagctagagaaggaattggtccaagcccgactgagtgaagctgagtctcagtgtgcattaaaagagatgcaggataaagtcttggatatagagaagaggaataactcccttcctgatgagaataatattgcaaggcttcaggaagaactcattgctgtgaaacttagagaagcagaagccattatgggtttgaaagaacttagacagcaagtcaaggatttagaggaacactggcagcgccacttagctcgtactactgggagatggaaagacccacccaagaaaaatgctatgaatgagttacaagatgaactgatgaccattcgacttagagaagctgaaacacaagcagaaataagagaaataaaacaaaggatgatggaaatggaaacacagaaccagatcaatagtaaccatcttcgaagagcagaacaagaggtgattagcctacaggagaaagtgcagtatctttctgcacagaacaaaggactccttactcaattaagtgaagcaaagcgtaaacaagcagagattgaatgcaagaataaggaagaagtgatggctgtgaggcttcgggaagcagatagcatagctgctgtggctgaactacgacaacacattgctgagcttgaaatccagaaagaagaaggaaagcttcaaggacagcttaacaagtctgattctaaccagtatattggggaactgaaagatcagatagcagagctgaatcatgagctcaggtgcctaaaaggccagaggggcttctcaggccaacctccttttgatggaatccacattgtcaaccatttaataggagatgatgaatcattccattcctccgatgaagattttatagataattccttacaggaaactggtgttggttttcctttgcacggaaaatctggttcgatgtctttggaccccgcagtggcagatggtagtgagagcgaaacagaagacagtgtgctggagaccagagagagcaaccaagtggttcaaaaggagcggcccccgagaagaagagagtcgtattcaaccactgtctgaccatcactgtgacctagactatggatttatttaagggatcagttatcatatgattagggctttttggaaataatttgtttcatatgtacatatatatatatatatatatatatatatatatatatatatatatatatatatatattttacagtcttatctgcctttttatctttgccaaatctttaccactgatttaccattgccataaagtagactggtatatggttaatgtgtaaaacaaggttctaacagtattactgaatattaatgtttcttgtttagaaaatgcaactatatctatatgtgggaaccattctgaagttcaaaactatggaaaattgaattttcttcagacaaaactgctcttgtgcaatattttatgctcagtgagcaaattgtgtatttatgtttatcaatttgttctcaaggtggctgtctacagacatttgaccaagacatacatctgatttaccttgtgtttttttttttattgttgtttttttttttaagacagagattcagtctgtcacccaggctggagtgctgtggtgtgatcttagctcactgcaacctccgcctcccaggttcaagcaattttcctgcctcagcctcccgagtacctgggactatatgtgcgcaccaccacgcctggctaattttttgtatttttagtagagatggggtttcaccatgttggctgggctggtctcgaactcctaacctcaagtgatccacccgcctcagcctcccaaagtgctgggattacaggtgtgagccactgcacccagcctaccttgtctgtttgtgtctgggaggttttttttttctatttttattttttcatgaaaattattggtgggccactgaaagtccccacacacaaagcctttattctatataattttataaacacaaattcatgattatctgttttgagagttttaagttttgttttaatgtttaacttttatgtgcatatgatgcttccatgtgttggttactagagtagtaggttaactacagacatactgttttgtttgtacatatttataaatctgtaccacctaacattgaacatcattttatatgaagaacatacatgttgcaaaatgactgctttcagcatctaacaggacttctgtaaataataggttaaaagttaaaaataaaacactaatgttttaagagctttagtattttgcttagcattcaatactagcagattcataactgatgcctgttcaaaaacactgttggtggaatcttttatttcatgtgtatgtaactactaaatttttcatgactgaagaggttatagcacatattagttagtgctaatattctactgaatataatttaagcaatgggctaaggtccaagaaacatagcaattatatctacaaataatctattaacatgatcttcagaatctctagtttttgttctatagataaagccataagaatcctttcctaccagtttttcctatatcatttagatattatttcctagaaaacaggatgcctgtcactgataagttagagaaaacacccaaattatacttgcactatggaaagacgacatgaatatgttgactgggtactttctactctcatttttgttagcctatgaaaaaaaaaacattttgttaacctatggaaaaagccttggaagagaaaagtagaattcagtatgtatggaggcacagttttgcacagtatgttgcatgcggcaaaaaaaaaaatatgaaatagagtagaagagataaaagtagttttctgtcaaaaactaggagttaagccttcaggtacgttgtggactaggctttccagcaaggtttttgtagctatttgcttgcctgagaaatgaaattgaaaacatacccctgcgtgtctgtccttaacaccaggctaaattctggtggttaaataacttatctgtggttacaagctagtaagtagcagagccaagattttttttaatatagagacaggtgtctcactgtgttgtccaggctggtcttgaactcctggcctcaagtggtcctcctgcctcagcctcccaaagtgtggggattacaggtgcgagctactgctcccagcaagagccaagattttaacctagatctccctaacaccaaagcttttgaaagggttcagctgaagaatttctcaattctctgttttgttttgtttttatttttgttttttgagatgaagtctcactgcactcaggctggagtgcagtggtacgatcttggctcactgcaacctccacctcccgggttcaagcgattctcctgcctcagctcccaagtagctgggactacaggtgcacgccaccacaactggctaatttttgtatttttagtagagatggggtttcgctatgttggccaggctgatctcaaacttctgacctcaggtgatccacccactttgacctcccaaagtgctgggattactagacgtaagccaccacacctggcaattccctgcttttttgacaacataatttaaatgaatttgatttaaatgtttcctgaatatatatacagatagatagatgagtatcaacacttacaagctctacacagtctgtgagtgtagatactacttactcgtaggattgtcttgtgctttggggatttttgttcggagttctatactatcagcccagaaagctatcaatgtccaagtgaagtgagatgatgcatggccacattattgctttgaccctcacatcaagaaatctgaatgatctggaccaggggtcagcaaagtgcagcctacaagctattgcagtcagctgccagtttttacttttttaaatagttgggaaaaaaattccaaagactgatattttgtgtcacatgaaaattatatgaaattcaaattgcagtgtccataagtaaagttttattggggcacacccatgctcacccttttaaatattgtgtatggccgctttgtgctacagtagcagagttgagtcattgcaagggagaccatatggcccaggtagcctaaaatatttatctgatcctttacagaaaaagtttgctaacccttgataacagatactctaaaatgcaggtttttcttcttcaatttagctcagtggtaggtcaaattgtttgcttttgctattaagcttatgatttggaaacgtaaaatttcatggtggatattataatcagaactattcaaaagggaaaacatgccagccattttatatgatgaagtctcttattccatagaaaactgtagaaagatatctgtatctttggagaaggagaaagaaaggaaagaactccagaatcccatcactgttctttaaagcatgcatcacagacctttaagagtatgctttattgagtaaaggttatagtttgactttaagataagaaattttctagaatcatgtttgaattctaaacgttgatattcttaagtaccatttctgattaggaattaggtgttaaaagaatcatggtaaggaaaccttaatccgttgaaactcttgtaatttggacaacaaaaaactagaaataacactcttttctttgaagatctgattcaaatacaccttagaagagtttaaaatctcccaggcctttaggatgcaataggatattatgccttggtttgactccttttcattcaaattaatgtaagtatgttgttcaagactttgaagcattgaacaatgttctcgatgaattttatttaattactgacattttgtgatatttgaatgttattaagttaaacacttagagttgagtatattcaggggagaaaagtgaatgaaatgactcataataagaaagcttgctcccttgggacagttaccttcctggtgtcattgccatcctgggttgacaagctgagagtagaggatacaagtgatgtgaataaaatggaaattgcattgagttaatgaaacagacacttttagatcaagaaaacacttcaacagcctttttagaaattggcacactcctatagtcccacctactcaagaggctgaggcaggaggatcactagagtacaggagtttggggctgtagtgtgtaatgatcacacctgtgaaagaataaccactgcactccaacctgggcaacatggcaagaccctgtctctttaaaaaaaaaaaaaatcctgggaaaagaagtcaaagatgtaattttacacaatttctgcccccaccgtcttttttgtttgtttgtttgtttgtttgttttttggctagatactaggtattcagtgtttgctgagatgacaggtttctactaatcagccagttatttgggaaggagaaaaactttttctcaattctgaacatgatgcagcttgttaactctccagatagtgcctttgattgaaaataaggtctacacctttttaagtatattatttctgacatcgtaagaaataatttagttccttattctcatttatcttttatataatgtactgtaatattcaggtttaaagtaaaataagtatatgaattatattttggtaaaactgtttcatgaccagcttgattaatgtatattaacataccagacattgacagcgaacatttcatgaaatcagttatttacttaccaaaacaatgagttttagcttgaaagttactgctgctctattattaacaaaactaagttttaaaaattaattttaattctactgaagtttaatttctggttaaaggtatagtggatgctatttatgatccaagtttttacatcacatccattgatctgcctatcccttctttttactccaaagaggagatgatagaataatacattcagtatactaccaagaagcaaagttataatgtgtttcattttgctattggaattttttattgtaacattgtatgataacctgaaatgtttacttgtgccttttctttaaacaattaaagttttccattttataagaa

SEQ ID NO: 119 - RNA, 1758 nt, unidentified
  source 1..1758: mol_type = other RNA
cttgcaccttctcttcctttacaagaagattttgtttatcactggaaggcaattacccattactacatagagacttcagatgataaagccccagtgaccgatacaaatattccatcgcatctggaacagatgttagatatactggttcaagaagaaaatgaacgggaatctggagagacagggccatgtatggaatatttgcttcatcacaagatcttggaaacattatataccttggggaaagctgattgtcctccgggaatgaaacagcaggttttggttttctatacgaaacttctgggaagaatccggcagccactacttccacacattaacgtgcacaggccagtgcagaaattaattagactctgtggtgaagtcctagcaacaccaacagaaaatgaagagattcagtttctttgcattgtgtgtgcgaagctgaaacaggacccctacctggttaactttttcctagagaataagatgaaatcattggcttccaaaggagtaccaaatgtaatttcagaagatacattaaaaggtcaggattccttgtcaacagatacaggacagtcccgtcaaccagaggaactatctggtgctactggaatggagcaaacagaattggaagatgagcctcctcatcagatggatcacctgtccacaagcttggataacctcagtgtcacctcactgccagaggcctcggttgtttgtccaaatcaggattacaatttagtgaattctttgttaaatcttactagaagtcctgatggcagaatagctgtgaaggcatgtgaaggcttgatgctgttagtaagtttgccagagcctgcggctgcaaagtgccttacacagagcacttgcttgtgtgaactactgacagacagacttgcctccctgtacaaggccctacctcagtcagtggatccgttagatattgaaaccgtggaagcaattaactggggcttggactcatatagtcataaagaagatgcttcagcatttccaggaaaacgagccttaatttcatttctttcctggtttgattattgtgatcagctcattaaggaagcccaaaagactgctgctgttgctcttgccaaagctgttcatgaaagatttttcattggtgttatggaacctcaattaatgcaaacttctgagatgggtattctcacatccactgctctgcttcatcgcatcgttcggcaagtgacctctgatgttttgcttcaagaaatggtgttttttatccttggagaacagagggaaccagaaactctggcagaaatcagcagacatcctttaaggcataggttaattgaacattgtgatcacatatctgatgagataagcataatgacattacgaatgtttgaacatcttttacaaaaacccaatgagcacattctttacaacttggtcttgagaaatcttgaagaaagaaattatacagaatataaacctttgtgcccagaagataaagatgtggtagaaaatggattgatagcaggagcagtagatctggaagaagatccattatttactgacatttcaccagaaaacactttgccaaaccaagagtggcttagttcttcacctcctgctactccagaccaccccaaaaatgatggaaaaactgaagttcataaaattgtaaatagttttctctgtctggtaccggatgacgcaaaatcctcctaccatgttgagggcacaggatatgacacttacctccgagacgctcataggcag

SEQ ID NO: 120 - RNA, 968 nt, unidentified
  source 1..968: mol_type = other RNA
cttgcaccttctcttcctttacaagaagattttgtttatcactggaaggcaattacccattactacatagagacttcagatgataaagccccagtgaccgatacaaatattccatcgcatctggaacagatgttagatatactggttcaagaagaaaatgaacgggaatctggagagacagggccatgtatggaatatttgcttcatcacaagatcttggaaacattatataccttggggaaagctgattgtcctccgggaatgaaacagcaggttttggttttctatacgaaacttctgggaagaatccggcagccactacttccacacattaacgtgcacaggccagtgcagaaattaattagactctgtggtgaagtcctagcaacaccaacagaaaatgaagagattcagtttctttgcattgtgtgtgcgaagctgaaacaggacccctacctggttaactttttcctagagaataagatgaaatcattggcttccaaaggagtaccaaatgtaatttcagaagatacattaaaaggtcaggattccttgtcaacagatacaggacagtcccgtcaaccagaggaactatctggtgctactggaatggagcaaacagaattggaagatgagcctcctcatcagatggatcacctgtccacaagcttggataacctcagtgtcacctcactgccagaggcctcggttgtttgtccaaatcaggattacaatttagtgaattctttgttaaatcttactagaagtcctgatggcagaatagctgtgaaggcatgtgaaggcttgatgctgttagtaagtttgccagagcctgcggctgcaaagtgccttacacagagcacttgcttgtgtgaactactgacagacagacttgcctccctgtacaaggccctacctcagtcagtggatccgttagatattgaaaccgtggaagcaattaactgggg

SEQ ID NO: 121 - RNA, 562 nt, unidentified
  source 1..562: mol_type = other RNA
attttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcag

SEQ ID NO: 122 - RNA, 477 nt, unidentified
  source 1..477: mol_type = other RNA
attttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaag

SEQ ID NO: 123 - RNA, 106 nt, unidentified
  source 1..106: mol_type = other RNA
gcaaagacaagaaaggaagctgcaatctctctcgtgtggacagcacaacctgccttttcccggtggaagaaaaagcagtggagtattactttgcttctgatgcaag

SEQ ID NO: 124 - RNA, 4083 nt, unidentified
  source 1..4083: mol_type = other RNA
gtggcaaactctcctctcagttgatgaccttgtggagaaactggtcaagaggctggagttcactggggagctcaacaacacttacatcttctatacctcagacaatggctatcacacaggacagttttccttgccaatagacaagagacagctgtatgagtttgatatcaaagttccactgttggttcgaggacctgggatcaaaccaaatcagacaagcaagatgctggttgccaacattgacttgggtcctactattttggacattgctggctacgacctaaataagacacagatggatgggatgtccttattgcccattttgagaggtgccagtaacttgacctggcgatcagatgtcctggtggaataccaaggagaaggccgtaacgtcactgacccaacatgcccttccctgagtcctggcgtatctcaatgcttcccagactgtgtatgtgaagatgcttataacaatacctatgcctgtgtgaggacaatgtcagcattgtggaatttgcagtattgcgagtttgatgaccaggaggtgtttgtagaagtctataatctgactgcagacccagaccagatcactaacattgctaaaaccatagacccagagcttttaggaaagatgaactatcggttaatgatgttacagtcctgttctgggccaacctgtcgcactccaggggtttttgaccccggatacaggtttgacccccgtctcatgttcagcaatcgcggcagtgtcaggactcgaagattttccaaacatcttctgtagcgacctcacacagcctctgcagatggatccctgcacgcctctttctgatgaagtgattgtagtaggtgtctgtagctagtcttcaagaccacacctggaagagtttctgggctggctttaagtcctgtttgaaaaagcaacccagtcagctgacttcctcgtgcaatgtgttaaactgtgaactctgcccatgtgtcaggagtggctgtctctggtctcttcctttagctgacaaggacactcctgaggtctttgttctcactgtattctttttatcctggggccacagttcttgattattcctcttgtggttaaagactgaatttgtaaacccattcagataaatggcagtactttaggacacacacaaacacacagacacaccttttgatatgtaagcttgacctaaagtcaaaggacctgtgtagcatttcagattgagcacttcactatcaaaaatactaacatcacatggcttgaagagtaaccatcagagctgaatcatccaagtaagaacaagtaccattgttgattgataagtagagatacattttttatgatgttcatcacagtgtggtaaggttgcaaattcaaaacatgtcacccaagctctgttcatgtttttgtgaattctaggctggtgctgcactgaaatagagcagtaagcttgtgataaaggccaattccaggtagctcttgaaggtgatagccatctactttccagtggctgccaaccacagggagtgccagttaacactggaaggattaaggcaaggtcccttctcttgagactcccctctgagatctgaaaaatgaagtggcttaggaacatcagcagtgaagaactgccaagagttggtgaaggttgtctcttccgagggccttctgaagacagggctcttgaacagacaagtggaagggctgtaccagggataaaggaaagaagtgcctgtccagcagggagcttgaatttaagttccatgtatgaagtcattggctctatctgcatttttctgtcattctcttcatttgttttaaggtggaaaattttcttacagttgatgcaaagtatcaactactttaccctaccttctccccttttagatgggttcttcctgagttttggagtcttgtatgattatcagtattcccctgtcaaaatcaaatctattcaggtttcttcactgttgagaacacctaaatgtttttatttttgagaagtggggacagagtctcactatgtcacccaggctggagtgcaatggcatgatctcagctcactgcaaccttcgcctcctgggttcaagcgattctcctgcctccgcctcctgagtagctgggattataggcacgcaccaccacgcccagctaattttttgtatttttagtagagacagagtttcaccatgttggccaggctggtcttgaactcctgaccttgtgatccacccacctcggcctcccagagtgctgggattacaggcatgagccaccacgcttggctaagaacacctaaatttttatgtttcttggctcaaaaaccagttccatttctaatgttgtcctcacaagaaggctaattggtggtgagacagcaggggaggaggaagagctgtggtttgtaacttgttcaactcaggcaataagcgattttagctttatttaaagtcttctgtccagctttaagcactttgtaagacatggctgaaagtagcttttctatcagaattgcagatagtcatgttgggctaacagtcaattggatatattcctttacctcacatgaccccagcaactgtggtggtatctagaggtgaaacaggcaagtgaaatggacacctctgctgtgaatgttttagagaaggaaattcaaaaaatgttgtaactgaaagcactgttgaatatgggtatcggctttctttttcactttgactcttaacattatcagtcaacttccacattaatgaaagttgaccatagttatttccaaataaaaagaaaccaactcttaccaggtcttggactgtgatgtcatattattcagttttatgcttgttcctgagcagaactcataagagtgacatagtcagctgctgacggcacctcagccacgccactcttactcagttcagtgggtgtgcttgcgtggtaggatgtggtgcagccctctctacgctcttctatttttggtatatttcctatctaaccttcaaatagcttccaattctttttttcttggactggcttcattctgaatttgtgctaaaataatctttcataaagagacctcagtttatagcgtaacagactacacaatgcactgatgttttcataatgtttaagggacccactgcaagaagcttgctgcctccttttaattgtattcatttagattttgattttccatgttaagaaggtgaggtccatgttggtgcccttcagagtagagaaccatgtaaacattaggaatgaacagaggccttaggaatgaatagagagtttgccttatacaatttcctgttacaaagctctccctctcatgcaaagtagggaacaccttttgagcatctttgaatttgacaaatggtgctgttgcaaacactttttttttgagatgaagtctcgcggttgtcacccgggctggagtgcagtggcgtgatctcggctcactgcaacttccacctcctgggttccagcagttctcctgcctcagcctcccaagtagctgagattacaggcgcctgccaccccacctggctgatttttgtaattttagtagagacggggtttcaccatgttggccaggctgattaactcctgacctcaggtgatccacctttctcggcctcccaaagtgctgggattacgggtgtgagccaccgtgcccggcctgcaaacacattttaattgacaacactagggctgttgtacaaaatagtaatgatagccatggaagttttaccttattctgtgagaagtgttcttaaacttattaagtgtctaaactaaggtttagtgcttttttaaaggaaagttgtcccaggattcatcctaaagaaagcaaaagttaattcaactgatccaccaatggaattagatgggtagagttgggttcttgagttttaccaccacttagttcccactgaattttgtaacttcctgtgtttgcatcctctgttcctattctgcccttgctctgtgtcatctcagtcatttgacttagaaagtgcccttcaaaaggaccctgttcactgctgcacttttcaatgaattaaaatttatttctgttctagtgggaa

SEQ ID NO: 125 - RNA, 705 nt, unidentified
  source 1..705: mol_type = other RNA
gtggcaaactctcctctcagttgatgaccttgtggagaaactggtcaagaggctggagttcactggggagctcaacaacacttacatcttctatacctcagacaatggctatcacacaggacagttttccttgccaatagacaagagacagctgtatgagtttgatatcaaagttccactgttggttcgaggacctgggatcaaaccaaatcagacaagcaagatgctggttgccaacattgacttgggtcctactattttggacattgctggctacgacctaaataagacacagatggatgggatgtccttattgcccattttgagaggtgccagtaacttgacctggcgatcagatgtcctggtggaataccaaggagaaggccgtaacgtcactgacccaacatgcccttccctgagtcctggcgtatctcaatgcttcccagactgtgtatgtgaagatgcttataacaatacctatgcctgtgtgaggacaatgtcagcattgtggaatttgcagtattgcgagtttgatgaccaggaggtgtttgtagaagtctataatctgactgcagacccagaccagatcactaacattgctaaaaccatagacccagagcttttaggaaagatgaactatcggttaatgatgttacagtcctgttctgggccaacctgtcgcactccaggggtttttgaccccgg

SEQ ID NO: 126 - RNA, 168 nt, unidentified
  source 1..168: mol_type = other RNA
tgtaatcgctcaaattcctcttctcctgttgacaaacttaatcagcagcctcgtctaaccaaactgacacgaatgcgcactgataagaagagtgaatttttgaaagcattgaaaagagacagagtagaagaggaacatgaagatgaaagccgtgctggctcagagaag

SEQ ID NO: 127 - RNA, 267 nt, unidentified
  source 1..267: mol_type = other RNA
ccttccttgaatccagaagctggcaaacagcatcagccatgcagacctattgggacaccttctggagtatgggaaaacccgcctagtgccaagcaaccctccaagatgctagttatcaaaaaagtttccaaagaggatcctgctgctgccttctctgctgcattcacctcaccaggatctcaccatgcaaatgggaacaaattgtcatccgtggttccaagtgtctataagaacctggttcctaagcctgtaccacctccttccaag

SEQ ID NO: 128 - RNA, 631 nt, unidentified
  source 1..631: mol_type = other RNA
ccttgtcttcaactcccttttcctcgccgaccttgaaccacgccttttcctgcaacaaccccagcacccttccccgtggatcccagcccatgagtcctcgaactacaatggggcggaggaggcagcgaagaagggaacacaagagctcgttgtcgctagcctccagcacggtggggccgggcgggcagattgtgcacacggagaccacggaggtcgtgctctgtggagaccccctcagcggctttggcctccagctccagggcggcatcttcgccaccgagaccctgtcctccccacccctcgtgtgcttcatcgagcctgacagtccggctgagaggtgtgggctgctgcaggtgggggaccgtgtcctgtccatcaatggcattgccaccgaggacgggactatggaggaagccaaccagctcctgcgggacgccgcactggcccacaaggtcgtgctggaggtggagttcgatgtggcggagtccgtcatcccaagcagtggcaccttccacgtgaagctgcccaagaagcgcagcgtggagctgggcatcaccatcagctcggccagcaggaaacgaggggagcccttgatcatctccgacatcaagaaaggcagcgtggcacacag

SEQ ID NO: 129 - RNA, 260 nt, unidentified
  source 1..260: mol_type = other RNA
gtatttctgggaaaagccagcttctgtttgcactggtcttcacaactcgttacctggatctttttacttcatttatttcattgtataacacatctatgaaggttatctaccttgcctgctcctatgccacagtgtacctgatctacctgaaatttaaggcaacctacgatggaaatcatgataccttccgagtggagtttctggtggtccctgtgggaggcctctcatttttagttaatcacgatttctctcctcttgag

SEQ ID NO: 130 - RNA, 3541 nt, unidentified
  source 1..3541: mol_type = other RNA
agctctggtatcgagactttagtggaggagctctgctccagactgaaagaccttcagagtaagcaagaagagaagattcacaaaaagttagaggggtctccctctccagaggcagaattatcccctccagcaaaggatcaagtggaaatgtactatgaagcatttccaccactttctgagaaaccagtttgcctgcaagaaatcatgactgtgtggaacaagtctaaagtctgttcttactctagctcttcttcatcatccacagccccaccagctagcacagatacttcctctcctaaggactgcaacagtgaaagtgaagtcaccaaggaaagaagcagtgaagtacccaccactgtgcatgagaaaacccagagcaaaagcaaaaacgagaaggaaaacaaatttagtaatggcacaattgaagaaaagcctgctttgtacaaaaagcaaatccgacataaacctgaaggaaagattcgccctcgctcgtggtcttctggctccagtgaagcaggctcaagttccagtgggaatcagggagaattaaaagcatccatgaagtatgttaaagtaagacacaaggcacgagagattcgaaacaaaaaagggcggaatgggcaaagcaggctttctttgaagcacggtgaaaaggctgaaaggaacattcatactggaagtagtagcagtagcagcagtggttctgtcaaacagctgtgcaagcggggtaagagacctttaaaagaaatagggagaaaagatcctgggagcactgaaggaaaagacctgtacatggagaatagaaaggacacagagtataaagaggagcccttgtggtacaccgagccaattgctgaatattttgttcctctgagcagaaaaagtaaactagagaccacataccgaaacagacaggatacaagtgatctgacatcagaggcagtggaagaattgtctgaatcagtgcatggtctttgtatcagcaacaataatcttcataaaacatacctcgcagcaggtactttcattgatggtcattttgtagaaatgcctgcagttataaatgaggatattgacctcactgggacctcattatgttctctaccagaggacaataaatacctggatgatattcatctatcagaattaacgcacttctatgaagtggatattgatcaatccatgttggatcctggtgcctcagaaacaatgcaaggagaaagtcggattttgaatatgattcgacagaaaagcaaagagaacacagattttgaggcagaatgttgcatagtgttagatggtatggagttgcaaggggaacgtgcaatatggacagattctaccagctccgtaggtgctgagggcttattcctgcaggaccttggcaatctggctcagttttgggagtgctgttcatccagctccggtgatgctgatggggagagttttggaggagactctccagttagactctctcccatcttagacagcacagtgctcaattcacacctgcttgctggcaatcaagagctcttttcagatattaatgaaggatctggtataaactcttgtttttcagtgtttgaagtgcaatgcagtaattctgttttaccattttcttttgaaacactcaacttgggaaatgaaaatacagattctagtgctaatatgcttgggaaaacacagtctagattgctaatatggaccaaaaatagtgcctttgaagaaaatgaacactgttctaatctttcaacaagaacttgtagtccatggtcccattcagaagaaacacgttcagacaatgaaacattaaatattcagtttgaagaatccacacagtttaatgccgaagatattaattatgtagttcctagagtctcgtcaaattatgtagatgaagaacttctagattttttgcaagatgaaacttgccagcaaaacagtagaactttaggtgagattcctacattagttttcaaaaaaacatctaaactagaatccgtctgtggtattcagctagaacaaaaaacagaaaacaaaaattttgaaactacacaagtatgtaatgaaagtccacatggagatggctacagctcaggggttattaaagacatttggacaaagatggcagacacaaattctgtggctacagtagaaatagaaagaactgatgctgagttgttttcggcagatgtaaataactactgctgctgtctagatgctgaagctgaactggagacccttcaggagcctgataaggctgtgcggaggtcagagtaccatctgtgggagggacagaaagagagcctggagaaaagagcatttgcttctagtgagctatcaaacgtggatggtggtgattatacaacaccctctaaaccctgggatgtagcccaagataaagaaaacacattcattcttggaggagtttatggagaactcaaaaccttcaatagtgatggggagtgggcagtcgtaccacctagtcacacaaaaggaagtctgttacagtgtgcagcttctgatgttgtgacgatagctggtacagatgtctttatgaccccaggaaacagttttgctcctgggcacaggcagttatggaaacccttcgtgtcatttgaacagaatgatcagccgaagagtggggaaaatgggttaaataagggattttcttttatcttccatgaagacttactaggagcttgtggcaactttcaagtcgaagatcctggacttgaatactcattttcttcctttgacttaagcaatccattttcacaagttcttcatgtagaatgctcatttgaacctgaagggattgcatctttcagccccagttttaaaccgaaatcaatcctctgttctgattcagacagtgaagtgtttcaccccaggatatgtggtgttgacagaacacaatacagggctattcggatctctcctcggactcactttcgcccaatttctgcatccgaactgtccccaggaggaggaagcgagtcagaatttgaatctgagaaagatgaagcaaatattcccattccttctcaagttgatatatttgaagatccgcaggcagatctcaaacctttggaagaagatgcagagaaagaaggccattactatggaaaatcagagcttgagtctggaaaattccttcccaggttaaaaaaatctgggatggaaaagagtgctcagacatcactggattcccaggaggaatcaactgggattctttcagtaggaaagcaaaatcagtgtttggaatgtagcatgaatgaatccctggaaatagatttagaaagctcagaagcaaattgtaaaataatggcacaatgcgaggaagaaattaataatttttgtggttgcaaagcaggttgtcagtttcctgcttatgaagataatccagtttcttcgggacagctggaagagttccctgtattgaacactgatatacaaggaatgaatagaagtcaagaaaaacagacctggtgggaaaaagccttgtactctcctctttttcctgcatcagagtgtgaag

SEQ ID NO: 131 - RNA, 2645 nt, unidentified
  source 1..2645: mol_type = other RNA
atttgccttgatgttttctctcctttcctcctatcccttctttctctttgttttacactctttgttgcggtgatgagttccttccagaggaaaaacaagaaatccagaaaaggtagagtacttcaatgtcacatcagtttttcttctccagtttgacacttaccagttcccatgctcatttgaattaaggttctgttttgctagctgcctcctggattgtaattctggtccccatctaaggccaggatcatcagacacctgaaaaataatcacgttcaccctgagagcaagggaagagagacttttctcaggataaaaggaaatccagacaaccaacactgcagagaacagagcctgatcccctaagcaacctgccgagaaccagagggaccaaatgaggaaccttactgcagtttatcatttgaaatgctttgtaggaagagggaggtagggaaaattattttgttcatgctgaaaaaagtgtttctttcctgtctctgggaatgagagtagatgagagatactgaatttactccttgatctggcggtttttattaggtggtagttcttttatgcattaaatatagatggtatataacctttcagatctaaaatgaaaaaagtcggagaaatggatctttccccatctctgcattcccttgtactccttaaaaagtacaagtacattctccgacagtgccctcttctggctaagacattctacatctttttttaattcattactctgtaatcttggaacttgtattttaacacatctgaagatgaaagccttcagaaatctctaatttgcttctatgtaaactcccagattccacccttgtgcaaactaataattaggctgaaatattgcaaaatgattcctccggaagtgataacctcatgattttatgaggatagtcttagctgtcctaactctgagaaataaccaactgtttgttatctaagggccatctctagcttttttatgtgcacatttaaggacaccatggtgttctccctccactgtgactagtggggacctttgaccacctcagaagcaagggggcaaatatatcgagagagaccttaaagccaggagacttgatgatctaccatgctttcaaataattagacatgctctgggaagcttggaggttactccttcctctgccttcttggagagcagacagatgtaagctatccctgatactcttacctgcttactagaacttgaataatgcacctttggaatagggcttttgtcatttctcttccagaaggagagtcatgctatgtggtgccaagaggaaatttgatggctcagctcactaataaatcttgtggacaaacttaatttcttcattccatagataaagaactgtgaatcctattgaagggtctttttatgttctttctggcttcccaagtcagtaaggtacctcaaaccagggagataaaagtgcctgctttgagcattgctacccaggtgctgaaggtgaacatgggtggaatctaggattttggaagcagacaatggcttttagctaagcgcattctcaagctcagtgacatctaggtctcagatagacttgagcccttgtcatgagactggtctggtttatggcatggtctgtgagcctacttttttgcttctttgagtccaggcccagtatctttgtgctgtcctttgcaatgctgagatgtggcattcccaaactttcttatagaagtttataaaaatagctagaaataaatatattgccttctatttccaaatattagacagataatccttgaaaagttgtaaatttgaattacattggcttagtttccccttttctgcctttcttgtcccctctaaaagcttaatgtgataaactgcagtaagccttcatcctcctgtgtcttttcccatcttcattgatctctctgcctttcctggaaagttgctcagatgactcaggcactgttctctgcagagtttgctatctgggtttatttttctgatgtgagcctttcttttcacttgctctccaggtgagcctggcatttgctaggaaccagagtggaaccggcaaagtggccagcaacagagtcctgatccaaatggagaaagggaaccaggcgtacctcaacctggagtgggaaacttgataggagtttgaatcagcttgggtactgacaaatattgcttcaggaaattctcttcagacccgaattgtgattcaggcaggagctgtgcccatcttcatagagttgctcagctcagagtttgaagatgtccaggaacaggcagtctgggctcttggcaacattgctggagatagtaccatgtgcagggactatgtcttagactgcaatatccttccccctcttttgcagttattttcaaagcaaaaccgcctgaccatgacccggaatgcagtatgggctttgtctaatctctgtagagggaaaagtccacctccagaatttgcaaaggtttctccatgtctgaatgtgctttcctggttgctgtttgtcagtgacactgatgtactggctgatgcctgctgggccctctcatatctatcagatggacccaatgataaaattcaagcggtcatcgatgcgggagtatgtaggagacttgtggaactgctgat

SEQ ID NO: 132 - RNA, 3183 nt, unidentified
  source 1..3183: mol_type = other RNA
ccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacctgaccatcgggggccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaggagaagcacggtgtcatccaccgcgacgtcaagccctccaacatcctgctggacgagcggggccagatcaagctctgcgacttcggcatcagcggccgcctggtggactccaaagccaagacgcggagcgccggctgtgccgcctacatggcacccgagcgcattgaccccccagaccccaccaagccggactatgacatccgggccgacgtatggagcctgggcatctcgttggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtagctgcttggcggcggccagccccacagggggccaggggcatggccacaggcccccctccccacttggccacccagctgcctgccaggggagacctgggacctggacggccacctaggactgaggacagagagtggggggtgcccacccaccccccccgccccgggcctaccaagcccccgcccttcccaccccggggtcagccggccgtgtgcgtcccccgacagacactgtgaacggaagacagcaggccgcgatcagagtcgctgttcattcagccgcagcctctgggccggggcggcccccaggggccaggagagagccctggagtcccgcagccaccatgcacgctcccagcgtgctgtgtccttcgccactcccacgcgcccgttcctcttccgtcgccctctgtcccctgctctacctctctgtccttgtctggctctcccgtcaccctccctgcctctgtctctcttctggcctgagcctgggcccagccacctcctgacgggtcccctgggtctgcataggtctcccatggcgcaatgagtcagtggcccccagccaggcagtgtgggcattgccactgcggctggacggggctgcgcgctcgcgctctctctctctctctctctctctctttgatctcagggggtcctttttggagtttattgtattttattgtacttggtggggtgtttggggtgggggcggaggagagcttgttctcgtggggttgtcggtaccttcagaaacttttaccaaagtcacgattagctgcttgtggtggggccccaaccgccctcgggcactggggagctgggctggggctgctgctctggggtctccgggggccacagcttggggtgagttgaagacctcaggggatgtggaggggtctgcggggccctggccgcacaggatggccttcagggaaggtggtcttggggcatggtgcagagcaggtgaccggagggaatcggtgacggagcggggccaagggaggggtccggagggagtcagggatggagggcagagggagtggatgtgggggtttgaggacgtgtgacaagctccagcaggggtgggggccgggctgagggtgggggtgcgaggtggtcactcccatcgtgcccctggccgtccctccactcacccacacctggcccagtccacgttgaggtccaggactgggaaggaccgggtgagtgcaccggggacccaggccaggtgccccccggagcctgctggggtggccagagcaggagggggtgtgtttcctttttgtgggtgttgcatgcaaatcaagtggacaagaaaaaataacaaaacaaaaaacaagaaaaaaaaaacacaaaaccccgtaaaatcacaaagaaaatccaacaccaaaggcgcagaagccggctggccgtggtgggggcagcgtaggcgtagcatccctctcctctcacttagcctgttgactcttgttattatcatgatattcacaaaacgccgcatgtttaaaaagtcatagatgtcatcttctctctgcccccagggaggaaagccaccttctcttgccccttggcccctttgtcaggggccaggggtctgccgggtgggggtgccaacaggcctggccctttcctcccctgcatccagccatgggggcctctgcgattgccggaaggttgcatggctggtcccagggccagcacaggcccgaggccgggctgcctggttttatttttatttaactttattttctgttttatgagtgtgtgtccgcccacccccacccccttcagtgttaagtggggagccctgggggagtctctcctgcctcccagcctctcccaagacctcccccctcgtcaccagccatccctctggaccaggcagagggcggaccgggtgggcaggggcctgagggtggctcgggccagcccaccagccaatggacccctcctcaggccgccagtgtcgccctgcccctttttaaaacaaaatgccctcgtttgtaaacccttagacgcttgagaataaaccccttccttttcttcca

SEQ ID NO: 133 - RNA, 2844 nt, unidentified
  source 1..2844: mol_type = other RNA
gagatctccttcttcttcctggagccctaccgctcgtcgtcctgcacctcctattcttcctgcctgggctgcttggcagaccagggctgtggctggtgcctgaccagtgccacctgccacctgcgccagggcggagcccattgcggggatgacggggctggtgggtccctgctggtgctggtgcctaccctctgcccactctgcgaggagcatcgggactgccacgcctgcacccaggaccccttctgtgagtggcatcagagcaccagccgcaaaggggacgcggcatgcagccggcggggccggggtcggggtgccctgaagagtccagaggagtgtcccccgctctgcagccagcgactgacctgtgaggactgcctggccaactctagccagtgcgcctggtgccagtccacccacacctgcttcctgtttgctgcctacttggcccggtacccacacgggggctgtcgaggctgggacgacagtgtacactcggagccacggtgccggagctgcgatggcttcctgacctgccatgagtgtctgcagagccacgagtgtggctggtgtggcaatgaggacaaccccacactgggacggtgcctacagggggacttctcagggcccctcggtgggggtaactgctccctgtgggtgggggagggcctggggcttcccgtggccctccctgcccgctgggcatacgcccgctgtcctgacgtggatgagtgtcgcctgggcctggcccggtgccacccgcgggcgacctgcctgaacacgcccctcagctacgagtgtcactgccagcggggctaccagggtgatggcatctcacactgcaaccgcacgtgcttggaggactgtggccatggtgtgtgcagtggccccccggactttacctgcgtgtgtgacctaggctggacatcagacctgccccctcccacacccgccccgggtccgccagccccccgctgctcccgggactgtggctgcagcttccacagccactgccgcaagcggggccctggcttctgcgacgagtgccaggactggacatggggggagcactgcgaacgatgccggcccggcagcttcggcaacgccacaggctctaggggctgccggccctgccagtgcaacgggcacggggacccacgccgtggccactgcgacaacctcagtgggctctgcttctgccaggaccacaccgagggtgcccactgccagctctgctccccaggctattatggggatccccgggccggtggttcctgctttcgggagtgtgggggtcgcgccctcctcaccaacgtgtcctcagtggcactgggctcacgccgggtcggggggctgctgcctccaggtggcggggctgcaagagccgggcctggcctgtcctactgtgtgtgggttgtctcggccactgaggagctacagccctgtgctcccgggaccctctgtcccccactcaccctcaccttctcccccgacagcagcaccccctgcacgctgagctacgtcctggcgtttgatggattcccacgcttcctggacactggtgttgtccagtcggaccgcagcctcatagctgccttctgcggccagcgacgggacaggcccctcactgttcaggccctgtctgggctgctcgtgctgcactgggaggccaatggctcctcatcctggggcttcaatgcttcggtgggctctgcccgctgtgggtcagggggccccgggagctgtcccgtcccccaggaatgcgtgccccaggacggtgctgcaggtgcggggctctgccgatgtcctcagggctgggctggcccacactgccgcatggctctgtgtcctgagaactgcaatgcccacactggggcaggaacttgtaaccagagcctgggtgtgtgcatctgtgccgagggcttcgggggccccgactgcgccaccaagctggatggcgggcagctggtctgggagaccctcatggacagccgcctctcagccgacactgccagccgcttcctgcaccgcctgggccacaccatggtggatggacccgatgccaccttgtggatgtttgggggcctgggcctgccccaggggctgctgggaaacctgtacaggtactcagtgagtgagcggcggtggacacagatgctggcgggagccgaggacgggggcccaggcccatcgccccgctccttccatgcagccgcatatgtgcccgctggccgtggtgccatgtatctgctggggggacttaccgctggaggcgtcacccgtgatttctgggtcctcaacctcaccaccctgcaatggcggcaggagaaggccccccagaccgtggagctgccagccgttgctggtcacacccttactgcccgccgaggcctgtctctgctcctggtgggcggttactccccggaaaatggcttcaaccagcagctgctggagtaccagctggcaaccggcacctgggtgtcaggagcccagagtgggacaccccccacaggtctctatggtcactctgctgtctaccacgaggccaccgactccctctacgtgtttggggggttccgattccatgtggagctggcggccccatcccccgagctctactccctgcactgtcctgaccgcacctggagtctgctggccccttctcagggggcaaagcgagatcgtatgaggaatgtgcgtggctcatctcggggtctgggccaagttcctggggagcagcctgggtcatgggggttccgggaagtcaggaagaagatggctctgtgggctgctcttgctggtacaggaggtttcctggaggaaatctcacctcacctgaaggag

SEQ ID NO: 134 - RNA, 8033 nt, unidentified
  source 1..8033: mol_type = other RNA
gagatctccttcttcttcctggagccctaccgctcgtcgtcctgcacctcctattcttcctgcctgggctgcttggcagaccagggctgtggctggtgcctgaccagtgccacctgccacctgcgccagggcggagcccattgcggggatgacggggctggtgggtccctgctggtgctggtgcctaccctctgcccactctgcgaggagcatcgggactgccacgcctgcacccaggaccccttctgtgagtggcatcagagcaccagccgcaaaggggacgcggcatgcagccggcggggccggggtcggggtgccctgaagagtccagaggagtgtcccccgctctgcagccagcgactgacctgtgaggactgcctggccaactctagccagtgcgcctggtgccagtccacccacacctgcttcctgtttgctgcctacttggcccggtacccacacgggggctgtcgaggctgggacgacagtgtacactcggagccacggtgccggagctgcgatggcttcctgacctgccatgagtgtctgcagagccacgagtgtggctggtgtggcaatgaggacaaccccacactgggacggtgcctacagggggacttctcagggcccctcggtgggggtaactgctccctgtgggtgggggagggcctggggcttcccgtggccctccctgcccgctgggcatacgcccgctgtcctgacgtggatgagtgtcgcctgggcctggcccggtgccacccgcgggcgacctgcctgaacacgcccctcagctacgagtgtcactgccagcggggctaccagggtgatggcatctcacactgcaaccgcacgtgcttggaggactgtggccatggtgtgtgcagtggccccccggactttacctgcgtgtgtgacctaggctggacatcagacctgccccctcccacacccgccccgggtccgccagccccccgctgctcccgggactgtggctgcagcttccacagccactgccgcaagcggggccctggcttctgcgacgagtgccaggactggacatggggggagcactgcgaacgatgccggcccggcagcttcggcaacgccacaggctctaggggctgccggccctgccagtgcaacgggcacggggacccacgccgtggccactgcgacaacctcagtgggctctgcttctgccaggaccacaccgagggtgcccactgccagctctgctccccaggctattatggggatccccgggccggtggttcctgctttcgggagtgtgggggtcgcgccctcctcaccaacgtgtcctcagtggcactgggctcacgccgggtcggggggctgctgcctccaggtggcggggctgcaagagccgggcctggcctgtcctactgtgtgtgggttgtctcggccactgaggagctacagccctgtgctcccgggaccctctgtcccccactcaccctcaccttctcccccgacagcagcaccccctgcacgctgagctacgtcctggcgtttgatggattcccacgcttcctggacactggtgttgtccagtcggaccgcagcctcatagctgccttctgcggccagcgacgggacaggcccctcactgttcaggccctgtctgggctgctcgtgctgcactgggaggccaatggctcctcatcctggggcttcaatgcttcggtgggctctgcccgctgtgggtcagggggccccgggagctgtcccgtcccccaggaatgcgtgccccaggacggtgctgcaggtgcggggctctgccgatgtcctcagggctgggctggcccacactgccgcatggctctgtgtcctgagaactgcaatgcccacactggggcaggaacttgtaaccagagcctgggtgtgtgcatctgtgccgagggcttcgggggccccgactgcgccaccaagctggatggcgggcagctggtctgggagaccctcatggacagccgcctctcagccgacactgccagccgcttcctgcaccgcctgggccacaccatggtggatggacccgatgccaccttgtggatgtttgggggcctgggcctgccccaggggctgctgggaaacctgtacaggtactcagtgagtgagcggcggtggacacagatgctggcgggagccgaggacgggggcccaggcccatcgccccgctccttccatgcagccgcatatgtgcccgctggccgtggtgccatgtatctgctggggggacttaccgctggaggcgtcacccgtgatttctgggtcctcaacctcaccaccctgcaatggcggcaggagaaggccccccagaccgtggagctgccagccgttgctggtcacacccttactgcccgccgaggcctgtctctgctcctggtgggcggttactccccggaaaatggcttcaaccagcagctgctggagtaccagctggcaaccggcacctgggtgtcaggagcccagagtgggacaccccccacaggtctctatggtcactctgctgtctaccacgaggccaccgactccctctacgtgtttggggggttccgattccatgtggagctggcggccccatcccccgagctctactccctgcactgtcctgaccgcacctggagtctgctggccccttctcagggggcaaagcgagatcgtatgaggaatgtgcgtggctcatctcggggtctgggccaagttcctggggagcagcctgggtcatgggggttccgggaagtcaggaagaagatggctctgtgggctgctcttgctggtacaggaggtttcctggaggaaatctcacctcacctgaaggagccccgcccccggcttttccacgcctcagccctgttaggggacaccatggtggttcttggggggcgctcggaccctgacgagttcagcagcgacgttctgctctaccaggtcaactgcaatgcctggcttctgcccgacctcacccgctcggcctctgtggggcccccaatggaggagtctgtggcccatgctgtggcagcagtcgggagccgcctgtatatctctgggggtttcgggggagtggccctgggccgcctgctggcactgaccctgccccctgacccctgccgcctgctgtcctcacctgaagcttgtaaccagtctggggcctgcacctggtgccatggggcctgcttgtccggggatcaggcccacaggctgggctgcgggggctccccctgctccccaatgcctcgctccccggaggaatgtcgacgtctccggacctgcagtgagtgcctggcccgccatcctcggaccctgcaacctggagatggagaggcgtccaccccccgctgtaagtggtgtaccaactgccccgaaggtgcttgcattggacgcaatgggtcctgcacctctgagaatgactgtcggatcaaccagcgagaggtcttctgggcagggaactgctccgaggctgcgtgcggggctgctgactgcgagcagtgcacgcgggagggcaagtgcatgtggacgcggcagttcaagaggacaggggagacccgccgcatcctctccgtgcagcccacctatgactggacgtgcttcagccactctctgctgaatgtgtcccccatgccggtggaatcatcacccccactgccctgccccaccccttgtcacctcctacccaactgtacctcctgcctggactctaagggagcagatgggggctggcagcactgtgtttggagcagcagcctgcagcagtgtctgagcccttcctacctgcccctgcgatgtatggccggaggctgtgggcggctgctccggggacctgagagctgctccctgggctgtgctcaggcaactcagtgcgccttgtgcctgcggcgcccccattgcggctggtgtgcctgggggggccaggatgggggtggccgctgcatggagggtggactcagcggcccccgtgatgggctgacatgtgggcgtccgggggcctcctgggccttcctgtcctgcccccctgaggacgagtgtgcaaacgggcaccacgactgcaacgagacgcagaattgccacgaccagccccacggctatgagtgcagctgcaagaccggctataccatggacaacatgacagggctgtgccgccctgtgtgcgcccagggctgcgtgaacggctcatgtgtggagcccgaccactgccgctgccactttggctttgtgggccgcaactgctccacggaatgccgctgcaaccgccacagtgaatgcgctggtgttggggcgcgtgaccactgcttgctctgccgcaaccacaccaagggcagccactgtgagcagtgcctcccgctgtttgtgggttcagctgtcggaggcgggacctgccggccctgccacgccttttgtcgtggaaatagccacatctgcatctccaggaaggagttacaaatgtccaagggagagccaaagaagtactcactggacccagaggagattgaaaactgggtgacagagggtcctagtgaagacgaggccgtgtgcgtgaactgccagaataacagctatggggagaaatgcgagagctgcctgcagggctacttcctcctggacgggaagtgcaccaaatgccagtgtaatggccacgcggacacatgtaacgagcaggatgggacgggctgtccatgtcagaataacacagagacgggcacatgccagggcagctcccccagtgaccgtcgagactgctacaagtaccagtgcgccaagtgccgggaatcatttcacgggagtccgctgggcggccagcagtgctaccgcctcatctcggtggagcaggagtgctgcctggaccccacgtcccagaccaactgcttccatgagcccaaacgccgggcgctaggccccggccgcactgtcctctttggcgtgcagcccaaattcaccaacgtggacatccgcctgacgctggacgtgaccttcggggccgtggacctctatgtctccacctcctatgacaccttcgtggtccgtgtggcccctgacactggcgtccatactgtacacatccagccacccccagccccaccacctccaccaccccctgcagatggtgggccccggggggctggggatccaggaggagcaggggccagcagtgggccgggcgccccagcagagccacgggtacgggaggtatggccgcggggcctgattacctacgtgacggtgacggagccgtcggcagtgctggtggtccgcggcgtgcgggaccggctggtcatcacctacccacacgagcaccatgccctcaagtcgagccgcttctacctgctgctgctgggcgtgggagacccaagtgggcccggcgccaacggctcagccgactcgcagggcctgctcttcttccggcaggaccaggcccacattgacctgtttgtcttcttctccgtcttcttctcctgcttcttcctcttcctctcactctgtgtgctcctctggaaggccaagcaggctctggaccagcggcaggagcagcgccggcacttgcaggagatgaccaagatggccagccgccccttcgccaaggtcaccgtctgcttcccacctgaccctactgccccggcctccgcctggaagccggctgggctcccacctcccgccttccgccgctctgagcccttcctggcacccctgctgctgacaggggccggtgggccctggggacccatgggagggggctgctgcccaccagccatccccgccaccactgctgggctgcgagctgggcccatcactctcgagcccacagaagatggcatggctggcgtggccacactgctgctccagctgcctggcgggccccatgcacccaacggcgcctgcctggggtcagccctcgtcacactgcggcacaggctgcacgagtactgtgggggtggtgggggtgctgggggcagtgggcatgggactggtgcgggccggaagggactgttgagccaggacaacctcaccagcatgtccctctgacatgcccagggttctcatccacagcagctgggtcacctgatagggccgccctggacttggggtccctccacctgggggcccctggacactgtctacttggagaccactggcccccttcccccagggttgcccagatggggcctcctttgttctgcattcagcagctatttatcgagtacctactctgtcaggcactgtcataggcgtggggcaaagcaggaaccaagagacgaggttccctgatctcatgggacttaggttctggtgaagggagacaatcagtgcacatgcacacaccccacacgcatacacacatgaacacatgcacatgtgcacacacaagtaagatggcttcagagagggagaagcactgtgaggcctccagaggatgtggcagtgagggacgatggggtgaagtcagctgggcattcaaagaagctagactgagaacgcctgagaagaaccagctacgggaagagctttgggaagcaaaggcagaggccctggggtgggagcaggcttgttttattggaaggaccagaaaactggtaagtgtgacccagatcaagtgtgaggagatgaggctggggatagtcaggggctggatcacccagggccttgtgggccccacatagggttttgggttttattctcagggcaatgggaagctgttggatggtttgatgaaggggagtgacaggatccgatgtacctatttaagaatttaagagggtcgggtgcggtggctcatgcctataatcctagcactttgagaggccaaggtgggctgatcacaaggtcaagagttcgtgaccagcctggccaatatggtgaaaccccatgtctactaaaaatacaaaaattagctgggcatggtggcacacacttgtagtccgagctactcgggaggctgaggcagaagaatcgcttgaacccaggaggcggaggttgcagtgagctcagattgcgtcactgcactccaacctgggtgacagagcgagactccacctcaaaaaaaaaaaaaaaatttaagaggtcactcagttgtgctgtggagaatggaccggagggacaagaggggcagcagggatggtgggctggagtagggtgctggcaatgagggagtctggctcagatgtgggatgtgtatggaagaatataaatgatggtgtggatgtcagggtgagggaggagacaaaaccacgatgacccctagctttgtggcctgaactgtgggtggctgaggggatcgttaattgaatggggcagactgaggcttgtgaggaagatcagagtctggttcttgacatgagatgcccttcagacatctcttcactcaggtccaactagggatacagaaacactgaatatttcaacagcagaaattgaatggggggattgatagcgctggcgagggaagcagctggaaagagacagatggcaccctgagacagcccagaggtgaataggacccccaggctgcagggataaagctcagtggtggtgttacctcaccggggaccagggtcacacagcaaagctggaacaacagaggcgtgttgtgggggagcctcagaggggacaaaacctctgcctgagatcccaccccaggtgggcatgggggccactgaggttggggatgaaaatgccggtaccgtcagtgcacagccctgttccagacagtgctgcctggaagatttctgggctctcctgaggcgccaccccgcacctgagccacctccttggactcctgtcctctaccccttgaggacctccctcccttctaccctagctgtcttcttgaacttgggactctcctttcccaagacttccatcactagctcctggagggactggactttgcatcttcccttcgcgtggagcctcagtgtgagaggccctagccaatgcgtgcatgtcagaggtggtggggaccacatcagaagaagaggggggtgatgaaattaacaaataaaaagtatggggaaaaccca

SEQ ID NO: 135 - RNA, 4141 nt, unidentified
  source 1..4141: mol_type = other RNA
cttcctgatttctcccgattccttccttctccctggagcggccgacaatgtccaccacggtcaatgtagattcccttgcggaatatgagaagagccagatcaagagagccctggagctggggacggtgatgactgtgttcagcttccgcaagtccacccccgagcggagaaccgtccaggtgatcatggagacgcggcaggtggcctggagcaagaccgctgacaagatcgagggcttcttggatatcatggaaataaaagaaatccgcccagggaagaactccaaagatttcgagcgagcaaaagcagttcgccagaaagaagactgctgcttcaccatcctatatggcactcagttcgtcctcagcacgctcagcttggcagctgactctaaagaggatgcagttaactggctctctggcttgaaaatcttacaccaggaagcgatgaatgcgtccacgcccaccattatcgagagttggctgagaaagcagatatattctgtggatcaaaccagaagaaacagcatcagtctccgagagttgaagaccatcttgcccctgatcaactttaaagtgagcagtgccaagttccttaaagataagtttgtggaaataggagcacacaaagatgagctcagctttgaacagttccatctcttctataaaaaacttatgtttgaacagcaaaaatcgattctcgatgaattcaaaaaggattcgtccgtgttcatcctggggaacactgacaggccggatgcctctgctgtttacctgcatgacttccagaggtttctcatacatgaacagcaggagcattgggctcaggatctgaacaaagtccgtgagcggatgacaaagttcattgatgacaccatgcgtgaaactgctgagcctttcttgtttgtggatgagttcctcacgtacctgttttcacgagaaaacagcatctgggatgagaagtatgacgcggtggacatgcaggacatgaacaaccccctgtctcattactggatctcctcgtcacataacacgtaccttacaggtgaccagctgcggagcgagtcgtccccagaagcttacatccgctgcctgcgcatgggctgtcgctgcattgaactggactgctgggacgggcccgatgggaagccggtcatctaccatggctggacgcggactaccaagatcaagtttgacgacgtcgtgcaggccatcaaagaccacgcctttgttacctcgagcttcccagtgatcctgtccatcgaggagcactgcagcgtggagcaacagcgtcacatggccaaggccttcaaggaagtatttggcgacctgctgttgacgaagcccacggaggccagtgctgaccagctgccctcgcccagccagctgcgggagaagatcatcatcaagcataagaagctgggcccccgaggcgatgtggatgtcaacatggaggacaagaaggacgaacacaagcaacagggggagctgtacatgtgggattccattgaccagaaatggactcggcactactgcgccattgccgatgccaagctgtccttcagtgatgacattgaacagactatggaggaggaagtgccccaggatataccccctacagaactacattttggggagaaatggttccacaagaaggtggagaagaggacgagtgccgagaagttgctgcaggaatactgcatggagacggggggcaaggatggcaccttcctggttcgggagagcgagaccttccccaatgactacaccctgtccttctggcggtcaggccgggtccagcactgccggatccgctccaccatggagggcgggaccctgaaatactacttgactgacaacctcaccttcagcagcatctatgccctcatccagcactaccgcgagacgcacctgcgctgcgccgagttcgagctgcggctcacggaccctgtgcccaaccccaacccccacgagtccaagccgtggtactatgacagcctgagccgcggagaggcagaggacatgctgatgaggattccccgggacggggccttcctgatccggaagcgagaggggagcgactcctatgccatcaccttcagggctaggggcaaggtaaagcattgtcgcatcaaccgggacggccggcactttgtgctggggacctccgcctattttgagagtctggtggagctcgtcagttactacgagaagcattcactctaccgaaagatgagactgcgctaccccgtgacccccgagctcctggagcgctacaatatggaaagagatataaactccctctacgacgtcagcagaatgtatgtggatcccagtgaaatcaatccgtccatgcctcagagaaccgtgaaagctctgtatgactacaaagccaagcgaagcgatgagctgagcttctgccgtggtgccctcatccacaatgtctccaaggagcccgggggctggtggaaaggagactatggaaccaggatccagcagtacttcccatccaactacgtcgaggacatctcaactgcagacttcgaggagctagaaaagcagattattgaagacaatcccttagggtctctttgcagaggaatattggacctcaatacctataacgtcgtgaaagcccctcagggaaaaaaccagaagtcctttgtcttcatcctggagcccaagcagcagggcgatcctccggtggagtttgccacagacagggtggaggagctctttgagtggtttcagagcatccgagagatcacctggaagattgacaccaaggagaacaacatgaagtactgggagaagaaccagtccatcgccatcgagctctctgacctggttgtctactgcaaaccaaccagcaaaaccaaggacaacttagaaaatcctgacttccgagaaatccgctcctttgtggagacgaaggctgacagcatcatcagacagaagcccgtcgacctcctgaagtacaatcaaaagggcctgacccgcgtctacccaaagggacaaagagttgactcttcaaactacgaccccttccgcctctggctgtgcggttctcagatggtggcactcaatttccagacggcagataagtacatgcagatgaatcacgcattgttttctctcaatgggcgcacgggctacgttctgcagcctgagagcatgaggacagagaaatatgacccgatgccacccgagtcccagaggaagatcctgatgacgctgacagtcaaggttctcggtgctcgccatctccccaaacttggacgaagtattgcctgtccctttgtagaagtggagatctgtggagccgagtatgacaacaacaagttcaagacgacggttgtgaatgataatggcctcagccctatctgggctccaacacaggagaaggtgacatttgaaatttatgacccaaacctggcatttctgcgctttgtggtttatgaagaagatatgttcagcgatcccaactttcttgctcatgccacttaccccattaaagcagtcaaatcaggattcaggtccgttcctctgaagaatgggtacagcgaggacatagagctggcttccctcctggttttctgtgagatgcggccagtcctggagagcgaagaggaactttactcctcctgtcgccagctgaggaggcggcaagaagaactgaacaaccagctctttctgtatgacacacaccagaacttgcgcaatgccaaccgggatgccctggttaaagagttcagtgttaatgagaaccagctccagctgtaccaggagaaatgcaacaagaggttaagagagaagagagtcagcaacagcaagttttactcatagaagctggggtatgtgtgtaagggtattgtgtgtgtgcgcatgtgtgtttgcatgtaggagaacgtgccctattcacactctgggaagacgctaatctgtgacatcttttcttcaagcctgccatcaaggacatttcttaagacccaactggcatgagttggggtaatttcctattattttcatcttggacaactttcttaacttatattctttatagaggattccccaaaatgtgctcctcatttttggcctctcatgttccaaacctcattgaataaaagcaatgaaaaccttga

SEQ ID NO: 136 - RNA, 2117 nt, unidentified
  source 1..2117: mol_type = other RNA
gcggcagctcttttccttcttcctccacttcccctaccctccaccgtccgggagccgccgccaccgccgccgaggagtcaggaagttcaagatggccgccgcggagacccagtcgctacgggagcagccagagatggaagatgctaattctgaaaagagtataaatgaagaaaatggagaagtatcagaagaccagtctcaaaataagcacagtcgtcacaaaaaaaagaagcataaacacagaagtaaacataagaaacataaacattcctcagaagaagacaaggataaaaaacataaacataagcataaacataagaaacacaaaagaaaagagattattgatgcttctgataaagagggtatgtctccagcaaaaagaactaaacttgatgatttagctttgctagaagacttggaaaaacagagagccttgattaaggccgaacttgataatgagttaatggaaggaaaggtccagtctggtatggggctcattttgcaaggttatgagtctggctctgaagaagagggggaaattcatgaaaaggcaagaaatggaaataggtctagtactagatcttcaagtacaaaggggaaacttgaacttgtggacaataaaattactacaaagaaacgaagtaaaagcagatccaaagaacggactagacataggtctgataaaaagaaaagtaaggggggtattgaaatcgttaaagagaaaacaactaggagcaagtcaaaggagaggaaaaaatctaaaagcccatccaaaagaagtaagtctcaagatcaagcaaggaaatcaaaatcccctacccttagaaggcgatctcaagagaaaattggtaaggccagatctcctactgatgataaggttaaaattgaagataaaagtaaatcaaaagataggaaaaaatccccaattataaatgaaagtagaagtcgcgatcgaggtaaaaaatccagatccccagttgatttaagaggtaaatccaaagacagaaggtcacggtccaaagagagaaaatcaaaacggtctgaaactgataaagaaaagaagccaattaaatctccctctaaagatgcttcatctgggaaagaaaataggtcacccagcagaagacctggtcgtagtcctaaaagaagaagtttgtctccaaaaccacgtgataaatcaagaagaagcaggtctccacttttgaatgatagaagatctaagcagagcaaatccccctcgcggacactgtctcctgggagaagagccaagagccgatccttagaaagaaaacgacgagaaccagagaggagacgactttcttctccaagaacacgacctcgagatgatatcctcagtagacgtgaaagatcaaaagatgccagccccatcaatagatggtctccaacccgaagaagaagtagatctcccattagaaggaggtctcgttccccactcagacgtagcaggtctccaagaagaagaagcagatctcctcggagaagggacagaggtcggaggagcagatcacgcttgcgaaggcggtctcgatcacgcggtggtcgtagacgaaggagcagaagcaaagtaaaggaagataaatttaaaggaagtctttctgaaggaatgaaagttgagcaggaatcttcgtctgatgataaccttgaagactttgatgtagaggaagaagatgaagaagccctaatagaacagagaagaatccaaaggcaggcaattgttcagaaatataaataccttgctgaagatagcaacatgtctgtgccatctgaaccaagcagcccccagagcagtacgagaacacgatcaccatctccagatgacattctggagcgagtagctgctgatgttaaagagtatgaacgggaaaatgttgatacatttgaggcctcagtgaaagccaagcataatctaatgacagttgaacagaataatggttcatctcagaagaagttgttggcacctgatatgtttacagaatctgatgatatgtttgctgcgtattttgatagtgctcgtcttcgggccgctggcattggaaaagatttcaaagagaatcccaacctcagagataactggaccgatgcagaaggctattatc

SEQ ID NO: 137 - RNA, 590 nt, unidentified
  source 1..590: mol_type = other RNA
actctcagcgttctcatctctcttccttcaccatgaagctgatggacaaattccactcacccaaaatcaagagaacgccatcaaagaagggaaaaccagctgaggtgtccgtaaagattccagagaagcctgtgaacaaagaggcaacagacagatttctaccagagggctaccctctccccttggatctggagcagcaggcagtagaatttatgtccaccagtgctgtggcttccaggtctcaaaggcagaagaacctgagctggctggaggagaaagagaaggaagttgtcagtgccctgcgctactttaagaccattgtggacaaaatggcaattgataagaaggtactggagatgcttccagggtcagccagcaaggtgctggaggccatcttacccctggtgcagaacgatcctcgaattcagcacagctcagccctctcttcctgctatagccgagtgtaccaaagcctcgccaacctcattcgctggtctgaccaagtgatgctggaaggcgtgaactcagaagacaaggagatggtgacgactgtgaagggggtcatcaaggctgtgctggatggagtgaag

SEQ ID NO: 138 - RNA, 433 nt, unidentified
  source 1..433: mol_type = other RNA
actctcagcgttctcatctctcttccttcaccatgaagctgatggacaaattccactcacccaaaatcaagagaacgccatcaaagaagggaaaaccagctgaggtgtccgtaaagattccagagaagcctgtgaacaaagaggcaacagacagatttctaccagagggctaccctctccccttggatctggagcagcaggcagtagaatttatgtccaccagtgctgtggcttccaggtctcaaaggcagaagaacctgagctggctggaggagaaagagaaggaagttgtcagtgccctgcgctactttaagaccattgtggacaaaatggcaattgataagaaggtactggagatgcttccagggtcagccagcaaggtgctggaggccatcttacccctggtgcagaacgatcctcgaattcagcacag

SEQ ID NO: 139 - RNA, 490 nt, unidentified
  source 1..490: mol_type = other RNA
tccttctttcctttttgctggtagggccgggtggttgctgccgaaatgggcaagttcatgaaacctgggaaggtggtgcttgtcctggctggacgctactccggacgcaaagctgtcatcgtgaagaacattgatgatggcacctcagatcgcccctacagccatgctctggtggctggaattgaccgctacccccgcaaagtgacagctgccatgggcaagaagaagatcgccaagagatcaaagataaaatcttttgtgaaagtgtataactacaatcacctaatgcccacaaggtactctgtggatatccccttggacaaaactgtcgtcaataaggatgtcttcagagatcctgctcttaaacgcaaggcccgacgggaggccaaggtcaagtttgaagagagatacaagacaggcaagaacaagtggttcttccagaaactgcggttttagatgctttgttttgatcattaaaaattataaagaa

SEQ ID NO: 140 - RNA, 1378 nt, unidentified
  source 1..1378: mol_type = other RNA
actttataacaaagatgccgtcattgaatttctcttggacaaatctgcagaaaaggctcttgggaaggcagcatctcacattaaaagcattaagaatgtgacagagctgaagctttctgataatcctgcctgggaaggggataaaggaaacactaaaggtgacaagcacgatgacctccagcgggcgcgtttcatctgccccgttgtgggcctggagatgaacggccgacacaggttctgcttccttcggtgctgcggctgtgtgttttctgagcgagccttgaaagagataaaagcggaagtttgccacacgtgtggggctgccttccaggaggatgatgtcatcatgctcaatggcaccaaggaggatgtggacgtgctgaagacaaggatggaggagagaaggctgagagcgaagctggaaaagaaaacaaagaaacccaaggcagcagagtctgtttcaaaaccagatgtcagtgaagaagccccagggccatcaaaagttaagacagggaagcctgaagaagccagccttgattctagagagaagaaaaccaacttggctcccaaaagcacagcaatgaatgagagctcttctggaaaagctgggaagcctccgtgtggagccacaaagaggtccatcgctgacagtgaagaatcggaggcctacaagtccctctttaccactcacagctccgccaagcgctccaaggaggagtctgcccactgggtcacccacacgtcctactgcttctgaagcccgcactgccaccgctcctgccccagaaggttgtttagtttccacgtaggcaggtcgctttgtgcctctgagtgcgctgctgtgtgttctctctatagttctgtgtcataaagctgtcctggccagccttcaagctggtgtggccactcttgatgtgaggcgtgtcggttccaggggggacatgggaggggctgcacagtggcccgaggtcatgcttgcttccacctgcaggtgcatttggtcctttccatggccaggaagccctgtgggctgcactttttatgcttgcagtaacaagagactccagagtcctcaccggtgcagagttggcacatattaattaactaaaattctaatgatcttgctaccagcaataaatcaagtaggccaagtgaaactgggctttaaaaaggatggatttcaaatacactgtgcccactagaagcttcgaagggcctcgtccctctgctacagccctgggaggagccaggatccttgttggtctagctaaatactgttaggggagtgtgccccatctcatcatttcgaagatagcagagtcatagttgggcacccagtgattgggttcaaaaataaagctggtctgcctcttctctc

SEQ ID NO: 141 - RNA, 1701 nt, unidentified
  source 1..1701: mol_type = other RNA
aacaacagaaaataaggaaattctctctcttgaagataaagttgtagactttagagaaaaagactcatcttcgaatttatcttaccaaagtcatgactgctctggtgcttgtctgatgaaaatgccactgaacttgaagggagaaaaccctctgcagctgccaatcaaatgtcacttccaaagacgacatgcaaagacaaactctcattcttcagcactccacgtgagttataaaaccccttgtggaaggagtctacgaaacgtggaggaagtttttcgttacctgcttgagacagagtgtaactttttatttacagataacttttctttcaatacctatgttcagttggctcggaattacccaaagcaaaaagaagttgtttctgatgtggatattagcaatggagtggaatcagtgcccatttctttctgtaatgaaattgacagtagaaagctcccacagtttaagtacagaaagactgtgtggcctcgagcatataatctaaccaacttttccagcatgtttactgattcctgtgactgctctgagggctgcatagacataacaaaatgtgcatgtcttcaactgacagcaaggaatgccaaaacttcccccttgtcaagtgacaaaataaccactggatataaatataaaagactacagagacagattcctactggcatttatgaatgcagccttttgtgcaaatgtaatcgacaattgtgtcaaaaccgagttgtccaacatggtcctcaagtgaggttacaggtgttcaaaactgagcagaagggatggggtgtacgctgtctagatgacattgacagagggacatttgtttgcatttattcaggaagattactaagcagagctaacactgaaaaatcttatggtattgatgaaaacgggagagatgagaatactatgaaaaatatattttcaaaaaagaggaaattagaagttgcatgttcagattgtgaagttgaagttctcccattaggattggaaacacatcctagaactgctaaaactgagaaatgtccaccaaagttcagtaataatcccaaggagcttactgtggaaacgaaatatgataatatttcaagaattcaatatcattcagttattagagatcctgaatccaagacagccatttttcaacacaatgggaaaaaaatggaatttgtttcctcggagtctgtcactccagaagataatgatggatttaaaccaccccgagagcatctgaactctaaaaccaagggagcacaaaaggactcaagttcaaaccatgttgatgagtttgaagataatctgctgattgaatcagatgtgatagatataactaaatatagagaagaaactccaccaaggagcagatgtaaccaggcgaccacattggataatcagaatattaaaaaggcaattgaggttcaaattcagaaaccccaagagggacgatctacagcatgtcaaagacagcaggtattttgtgatgaagagttgctaagtgaaaccaagaatacttcatctgattctctaacaaagttcaataaagggaatgtgtttttattggatgccacaaaagaaggaaatgtcggccgcttccttaatcatagttgttgcccaaatctcttggtacagaatgtttttgtagaaacacacaacaggaattttccattggtggcattcttcaccaacag

SEQ ID NO: 142 - RNA, 651 nt, unidentified
  source 1..651: mol_type = other RNA
tggaagcattgacatttcctccttcttctggaaagtcattcatcatgggagcagatgaagcccttgaaagtgaactgggacttggagaactagcaggccttacggtggccaatgaagcagactcactaacttatgatatagcaaacaataaagatgcattgaggaagacatggaaccctaagtttacattgagaagtcactttgatggcatccgagcccttgctttccatcccattgagcctgttttgataacagcatcagaggatcacacattaaaaatgtggaatttacagaaaacagccccagccaaaaagagcacttctcttgatgtagaacctatctatacattcagagcccataaaggtccagtgctttgtgtggtaatgagcagcaatggtgagcagtgttacagtggtggtactgatggactgatccagggctggaataccactaatcccaacatcgacccctatgattcttatgatccttctgttttacgaggccctctgctaggccacacggatgcagtctggggtttggcttatagtgcagcacatcagcgtttgttgtcctgttcagcagatggcactctgcgtttatggaatacaactgaggttgctccagcactaagtgtatttaatgatactaaag

SEQ ID NO: 143 - RNA, 792 nt, unidentified
  source 1..792: mol_type = other RNA
tggaagcattgacatttcctccttcttctggaaagtcattcatcatgggagcagatgaagcccttgaaagtgaactgggacttggagaactagcaggccttacggtggccaatgaagcagactcactaacttatgatatagcaaacaataaagatgcattgaggaagacatggaaccctaagtttacattgagaagtcactttgatggcatccgagcccttgctttccatcccattgagcctgttttgataacagcatcagaggatcacacattaaaaatgtggaatttacagaaaacagccccagccaaaaagagcacttctcttgatgtagaacctatctatacattcagagcccataaaggtccagtgctttgtgtggtaatgagcagcaatggtgagcagtgttacagtggtggtactgatggactgatccagggctggaataccactaatcccaacatcgacccctatgattcttatgatccttctgttttacgaggccctctgctaggccacacggatgcagtctggggtttggcttatagtgcagcacatcagcgtttgttgtcctgttcagcagatggcactctgcgtttatggaatacaactgaggttgctccagcactaagtgtatttaatgatactaaagaactgggaatccctgcctctgtggatctagtgagcagtgacccgagccatatggtagcatcattcagcaagggatatacaagcatttttaacatggaaacacaacaacgcattctcactttagaatccaatgtagatacaa

SEQ ID NO: 144 - RNA, 646 nt, unidentified
  source 1..646: mol_type = other RNA
cctgtcctttcatcagagcacatggcagcctttggggtctttggtctctgccagatccatgcctttgtggattacctgcgcagcaagttgaatccacaacaatttgaagttcttttccggagcgtcatctctctggtaggctttgtccttctcaccgtgggagctctcctcatgctgacaggaaaaatatctccctggacggggcgtttctactcgctgctggatccctcttatgctaagaacaacatccccatcattgcttctgtgtctgagcatcagcccacaacctggtcctcatactattttgacctgcagctcctcgtcttcatgtttccaggtatgtggcctcgtgttctgaaatggccttgttcataagaatcacaatttgattccaaatttgcaagctaccctcattcgtgttttgtttttctttgcctgtttatgttgtgtaatacagaggacggctaagcaaattttctcatgtatgaagctgagcagaagtcagaagttcaacagttgtagatattggcaagttcagcctcacagttttctgttcatttgtcatttgtgttataagttaaaatttaacggtaatttttactccggttttgttttgttttgtttgagtacttctgtcgtactcctt

SEQ ID NO: 145 - RNA, 387 nt, unidentified
  source 1..387: mol_type = other RNA
agtttgaacaactgactcttgatggacacaaccttccttctctcgtctgtgtgataacaggcaaagggcctccgagggagtattacagccgcctcatccaccagaagcatttccagcacatccaggtctgcaccccttggctggaggccgaggactacccccgcttctagggtcggtggatctgggtgtctgtctgcacacgtcctgcagtggcctggacctgcccatgaaggtggtggacatgttcgggtgctgtttgcctgtgtgtgccgtgaacttcaagtggcaggagcagaacccgaatctttctggggatagcttcacagatccaccgctgaggaggaaacagtgcagagcgagctgcccacagtgaggccctgctcctgg

SEQ ID NO: 146 - RNA, 728 nt, unidentified
  source 1..728: mol_type = other RNA
attcttctctcttttctcactcccggtggcaaccggcttcgcaaccaaatctgtgaagttttggacacagacctcattaggcagcaggctgagcacagtgctgttgacatccaaggcctggccaactatgtcatcagtacgatgggaaagctgtgtgctcccgtgcgagataatgatatcagagagttaaaggctactggcaacatcgtggaggtgctgagacaaatattccatgtcctggacctcatgcaaatggacatggccaattttacaattatgagtctcagaccgcaccttcaacgccagttggtggaatatgagagaaccaagttccaggaaattttggaagaaactccaagtgctcttgatcagactacagaatggataaaagaatctgtaaatgaagaattattttctctttctgagagtgctttaactcctggggccgaaaatacctccaagccaagcctgagccctactttggtgctaaataatagttacttgaaactgttacagtgggattatcagaaaaaagaattaccagagacacttatgacagatggagcacgtcttcaggaactaacagaaaagctgaatcaattgaaaattattgcctgcctgtccctaattaccaacaacatggtgggtgctattacaggaggcctgcctgagcttgcaagcaggttaacaaggatttcagctgttctacttgaaggcatgaacaaaga

SEQ ID NO: 147 - RNA, 1557 nt, unidentified
  source 1..1557: mol_type = other RNA
acaaagacccgtatagttttgggaggacaacaatagcaaacctacagactgacaatgacctcctttggctgcacaccatctttgctgtcatttacctcttcctcactgtgggtttcatgcggcaccacactcagtccattaagtacaaagaggagaacctggtgaggcggaccctgttcatcacaggactccccagagatgccaggaaggagactgtggagagccacttccgggacgcgtatcccacgtgtgaggtggttgatgtgcagctgtgctacaacgtggccaaactgatctacctgtgcaaggagaaaaagaagactgagaagagcctgacctattacacaaacctgcaggtgaagacaggccagcggaccctcatcaaccccaagccctgtggccagttttgctgctgtgaagtgctgggctgtgagtgggaagacgccatctcttactacacacggatgaaggacaggctgctggagaggatcacagaggaagaacgccacgtccaggaccagcccctgggaatggccttcgtcaccttccaggagaagtccatggccacctacatcctgaaagatttcaatgcctgcaagtgtcagagccttcagtgcaaaggtgagccccagccgtcctcccatagcagggagctctatacctccaagtggacagtcacctttgctgctgaccctgaggacatctgctggaagaacctctctatccagggcctccgctggtggctacagtggctgggcatcaacttcaccctcttcctggggctatttttcctgaccacaccctccatcatcctgtccaccatggacaagtttaatgtcaccaaacccatccatgcgctgaataacccgatcatcagccagttcttccccaccctcctgctctggtccttctcggccctgctcccctccattgtctactactctacactgctggagtctcactggaccaagtcgggggaaaaccagatcatgatgaccaaagtctacatattcttgatcttcatggtgctgatcctgccctccctgggtctcaccagtctagattttttcttccggtggctctttgacaaaacttcctcggaggcctccatcaggttggagtgcgtcttcctgcctgaccagggtgccttctttgtgaactatgtcatcgcctcggccttcatcggcaatggcatggagctgctgcggctgccaggtctcatcctctataccttccgcatgatcatggccaagacggctgctgaccgcaggaatgtcaagcagaaccaggccttccagtacgagtttggagccatgtatgcatggatgctgtgtgtcttcactgtcatcgtggcctacagcatcacttgtcccatcatcgcgccatttggcctcatctacatcctgctcaagcacatggtggaccggcacaacctctacttcgtctacctcccagccaagctggagaaggggatccactttgccgctgtgaaccaggccttggcagcccccatcctgtgcctcttctggctctacttcttttccttcctgcgcctgg

SEQ ID NO: 148 - RNA, 500 nt, unidentified
  source 1..500: mol_type = other RNA
tctagattttttcttccggtggctctttgacaaaacttcctcggaggcctccatcaggttggagtgcgtcttcctgcctgaccagggtgccttctttgtgaactatgtcatcgcctcggccttcatcggcaatggcatggagctgctgcggctgccaggtctcatcctctataccttccgcatgatcatggccaagacggctgctgaccgcaggaatgtcaagcagaaccaggccttccagtacgagtttggagccatgtatgcatggatgctgtgtgtcttcactgtcatcgtggcctacagcatcacttgtcccatcatcgcgccatttggcctcatctacatcctgctcaagcacatggtggaccggcacaacctctacttcgtctacctcccagccaagctggagaaggggatccactttgccgctgtgaaccaggccttggcagcccccatcctgtgcctcttctggctctacttcttttccttcctgcgcctgg

SEQ ID NO: 149 - RNA, 1659 nt, unidentified
  source 1..1659: mol_type = other RNA
atttcaccttcaggaccttcctttcacccttccactcgccccagtttccaggagctaagaagggagacagctggcagcagccggacttcagtgagccaggcctcaggattgctcctggagaccccagtccagcctgctttctctcttcctaaaggagaacgcgaggttgtcactcactcagatgaaggaggtgtggcctctcttggtctggcccagcgagtaccattaagagaaaaccgagaaatgtcacataccagggacagccatgactcccacctgatgccctcccctgcccctgtggcccagcccttgcctggccatgtggtgccatgtccatcaccctttggacgggctcagcgtgtaccctccccaggccctccaactctgacctcatattcagtgttgcggcgtctcaccgttcaacctaaaacccggttcacacccatgccatcaacccccagagttcagcaggcccagtggctgcgtggtgtctcccctcagtcctgctctgaagatcctgccctgccctgggagcaggttgccgtccggttgtttgaccaggagagttgtataaggtcactggagggttctgggaaaccaccggtggccactccttctggaccccactctaacagaacccccagcctccaggaggtgaagattcaacgcatcggtatcctgcaacagctgttgagacaggaagtagaggggctggtagggggccagtgtgtccctcttaatggaggctcttctctggatatggttgaacttcagcccctgctgactgagatttctagaactctgaatgccacagagcataactctgggacttcccaccttcctggactgttaaaacactcagggctgccaaagccctgtcttccagaggagtgcggggaaccacagccctgccctccggcagagcctgggcccccagaggccttctgtaggagtgagcctgagataccagagccctccctccaggaacagcttgaagtaccagagccctaccctccagcagaacccaggcccctagagtcctgctgtaggagtgagcctgagataccggagtcctctcgccaggaacagcttgaggtacctgagccctgccctccagcagaacccaggcccctagagtcctactgtaggattgagcctgagataccggagtcctctcgccaggaacagcttgaggtacctgagccctgccctccagcagaacccgggccccttcagcccagcacccaggggcagtctggacccccagggccctgccctagggtagagctgggggcatcagagccctgcaccctggaacatagaagtctagagtccagtctaccaccctgctgcagtcagtgggctccagcaaccaccagcctgatcttctcttcccaacacccgctttgtgccagcccccctatctgctcactccagtctttgagacccccagcaggccaggcaggcctcagcaatctggcccctcgaaccctagccctgagggagcgcctcaaatcgtgtttaaccgccatccactgcttccacgaggctcgtctggacgatgagtgtgccttttacaccagccgagcccctccctcaggccccacccgggtctgcaccaaccctgtggctacattactcgaatggcaggatgccctg

SEQ ID NO: 150 - RNA, 6594 nt, unidentified
  source 1..6594: mol_type = other RNA
cttctccaatttgggaaatacctgctatatgaatgctattctacaatctctattttcactccagtcatttgcaaatgacttgcttaaacaaggtatcccatggaagaaaattccactcaatgcacttatcagacgctttgcacacttgcttgttaaaaaagatatctgtaattcagagaccaaaaaggatttactcaagaaggttaaaaatgccatttcagctacagcagagagattctctggttatatgcagaatgatgctcatgaatttttaagtcagtgtttggaccagctgaaagaagatatggaaaaattaaataaaacttggaagactgaacctgtttctggagaagaaaattcaccagatatttcagctaccagagcatacacttgccctgttattactaatttggagtttgaggttcagcactccatcatttgtaaagcatgtggagagattatccccaaaagagaacagtttaatgacctctctattgaccttcctcgtaggaaaaaaccactccctcctcgttcaattcaagattctcttgatcttttctttagggccgaagaactggagtattcttgtgagaagtgtggtgggaagtgtgctcttgtcaggcacaaatttaacaggcttcctagggtcctcattctccatttgaaacgatatagcttcaatgtggctctctcgcttaacaataagattgggcagcaagtcatcattccaagatacctgaccctgtcatctcattgcactgaaaatacaaaaccaccttttacccttggttggagtgcacatatggcaatttctagaccattgaaagcctctcaaatggtgaattcctgcatcaccagcccttctacaccttcaaagaaattcaccttcaaatccaagagctccttggctttatgccttgattcagacagtgaggatgagctaaaacgttctgtggccctcagccagagactttgtgaaatgttaggcaacgaacagcagcaggaagacctggaaaaagattcaaaattatgcccaatagagcctgacaagtctgaattggaaaactcaggatttgacagaatgagcgaagaagagcttctagcagctgtcttggagataagtaagagagatgcttcaccatctctgagtcatgaagatgatgataagccaactagcagcccagataccggatttgcagaagatgatattcaagaaatgccagaaaatccagacactatggaaactgagaagcccaaaacaatcacagagctggatcctgccagttttactgagataactaaagactgtgatgagaataaagaaaacaaaactccagaaggatctcagggagaagttgattggctccagcagtatgatatggagcgtgaaagggaagagcaagagcttcagcaggcactggctcagagccttcaagagcaagaggcttgggaacagaaagaagatgatgacctcaaaagagctaccgagttaagtcttcaagagtttaacaactcctttgtggatgcattgggttctgatgaggactctggaaatgaggatgtttttgatatggagtacacagaagctgaagctgaggaactgaaaagaaatgctgagacaggaaatctgcctcattcgtaccggctcatcagtgttgtcagtcacattggtagcacttcttcttcaggtcattacattagtgatgtatatgacattaagaagcaagcgtggtttacttacaatgacctggaggtatcaaaaatccaagaggctgccgtgcagagtgatcgagatcggagtggctacatcttcttttatatgcacaaggagatctttgatgagctgctggaaacagaaaagaactctcagtcacttagcacggaagtggggaagactacccgtcaggccttgtgaggaacaaactcctgggttggcagcatgcactgcatatttgttactgctgcccacctcacctttcctctgctgaaggagaatttggaattctacttgatgcgggagcaacaaacagctcagggccaaaccaaaagacaaaaattggagtaacgtagaatgctccatgctattttatggaaactttggtctcacatccgtagctgattatcctctttttctcctatgagtggcacttcttttgtcttaggaatacatgttgtaaatatatatctgtgtatgtgtgtatacacacacacagacacacacacacacacacgggatgaatggagccttaaagagttaggatgagccaccagaatatgcctgctcaaaattaatagcacagcagtttggagaagaaatgaaggtgtcaaagagtccattcacctgagaaatgtgtgaagacatacttatcagttggcttttagcttttatgttccttgagtagtttcactcaagtctgtaaccttttgtgtttccttattagtaaaattcactggaaagccagctcttcatgttacactaatgacagtttgttctctttgcaagagaggggcattactgtcacctgacttgaggagctgttttgttgttgttgttgtctgcaaatttcatgaatttgtgatgtctttgctgtttacatgcagtcccaagaaatggattgttggtgctttggaatatgttacagtcccacatttgatatttcttatatactttgttttctctaaggagatttcttcacacagtatgttcatcatatatcatcatcattattatggtggtaaagatagaatcttttttcttttttgtcattctgccatggagcagcattaccctaatggattgcaaccaaaactttaaacaagtagaaagataatatttctccaattgggactccccagcaggaatacttagggataaggaagaatgctagcatctctgtctctcaaacatagggaggataagaagagtgttcttctggtaaagctaaaattctggaccactgaagctaaaagccctattgcaagtatgaaattaagtacttgagctgtaggacaaaccttgggcatttaaccatttactgtctggctttgcccttaaaatagggttgcaattaaaatgtgattggcttaggtaatcccaaaaactaacaaataacaaaggtgcataatttatttatctactttttaggtgctctgagttgaggcaaagtagagcggcaacattaagtgctatgctagtcacttagctgacgtaaccagcttggttaagcagcttatgaaaccatataaagaattcttttgaggatggaattctgtccacaaaataattttgtgagcccagatatcattaggatcacacagagttaaatatagaaaaatgaaaccatcattatattctttcgtgttttttcttttattataaacaaggggattattctttagttctcagaggtagggacaaaaccacatcaggttttcagaaggaaaaaacatttaaaaacccaccatcacatgagagaatcacttgaacccaggaggcagaggttgcagtgagctgagatcgcatcattgcactgcagtctgagtgacagagtgagactccatctcattaaaaaaaacgaaaacaaaaacaaaaaaaacacaaaccatcatcacagaagatgcaacatcttttctgaaagattgccttaagagagctccagtcctactcttggaacttggatgtattctaatgtagtaaactattctaagttttcattctttgaattataaatggcctcagcagttttgctcaactactgataaatgctttgcctcctaccatctacctatataccttattgtaatgaatgttccaaaatggtagagtggtagaaaacgccagagtagtttagagcagaggaaaatatttgttttaaaactagctttaaagttttgtttcatttctaccaggagcctctttggtttggtttgattttgcttggaaataattggttttcttataaatgagtgaagcggtgataaaattctttggctagttattaattcttttacgtgtctttgcatttgagaggcactgttaaaaattattgggaaagatttaaagtgcaggctgcaattaaaacatggagaaaagtagaaataatgccataagtctagattgcctcatgaagcagcctcatttgaattgctttcatagctttcatgtgtgtatggtttagaagtacacttacctcagaaacctgatttgatcttcatgtcttgggcaggagttgttgaaaataggttattggaatatataagttatctcactgctcatgtacttgcatgtttgggactcaaattttacttgatgtctgtccattcttgtggccctatcagccttctccttcctttactcctttaatctacttctttgactaccagtggagatttcagctggactatgttgatggggtttgttgttgtttttgagctggctgtatatattttaaaattataaatagataatatattattttttgcacattgtgatcagtttgcccagaattggggatggggcagttagcatgttggggccaggaagggacaagttagataaggactgagtgcttccgttgcaggcagtttgcacaatacctaattaaccttcctattgaaaataccaaatgtgtggttacattactttaaatgacattgaattggaacttgatggcaattagttctagatacaaccttttgatctttgttggaattttaagggaaaaatgaactaaacttcatatttgttgattttaatcacctaatagtactgaaggttggaaagttgcatgtggctggatgctgttaatttctttaatagccatgactataatttaagcctatgagaagatagactgtttcgtagggatatattcacatgtgtgtgcacactcatgagttttgtctcagcaggatagaataagcaaatccatgaactggtcttctattaatactttgattctaaaaattatttgtttacttgctatggtctgttcattctgggcctaaccttaaaggctcaataacaaatacaacaaatgtaaatatgtttacattttaagacatatgcagtggttcttacagatcagttaattaactccagaaagcaaatgttagactacacatttatttttctccttgatcaagtataaaattctgaaacagaggctttaaaatttaaaacctcagcaaaataatccatgaatttactgattcttctgtatcgtgtgtagttaccattatgtaactatacacacatacagctacggatataatgaaaattatctgcaaacacctaaattaaagagaaaaaaaagtagtttgtaaactcatttgtgatctactgaaaagaggttatattaaagcaaattaaaatattttccttctctgtcctctgaaatgactgcagtagatactcttagttatcccatttagggtggttggagggcctttttaatttaattgaccccccgaggtggtcttttgttttaaacagggggcaacagctggatctccaagtacatatggattttgtatatataggagatttttagaaaaatcaacaacctaacaccagattcaagtcacataaaagtgttccaataaaatggatggatgtctttttcctccccattttgctttatactgagtaatgcactcttgcaagtgtatacaaaaactaaatagacttctgccctccaacatctttttattgcattacttcaaaatcctaattttgtctctactgatatgtcttattaacatctgaaaaaatatatattttttattggaaaattctaatttgttaggccttgaaagttttgtgacaattattttgctgtgtttaaccacaatcattcaccttatggcatgctttgattactagacttcaggcagtctcattcattgtatcatacttacacacaattagtaagttgtctccatgtgtgctatatgtctgaggtgtatggagtttttatttaaaaagtgtgccagtctgaatataagcatttgattttgtaacattggacttttcttaaaagtacagaggttcaaagtataggtatgtccattggcataagaatagagtgggtgagggtctggaccaggttccaggttggtccagtcagatgccagaacaaagaagaacagtcaactaaactggtctacttcaaaaatagtgggcctgtgtgaagagtgagaccacatgtgggtgtgcacacctcttgtccccaggtttccctccctttgagcttttctttccctccctaacttctctggcctattgtcattgttgtttctttaaacttaaggagagaaaaacaaaaatgagattcctacactttgcctaattgagccactaccaggttttctggcagctgtggccacattatttgtgagatgattttttttctttatgttcagagtgacttttgattctgattctttatgttttgtatggagcggcacttttatctgtgttttagcagaactgttcctctgtatcctttacggtttttctttgtttttgtttcctttttaaattatgcatagagtttttttgtgtgtatgaaattaaagcctttattaaccttc

SEQ ID NO: 151 - RNA, 2619 nt, unidentified
  source 1..2619: mol_type = other RNA
atggtaagaaaaaaaagcttattcgtatgcttgtttttatggctgcttttggtttctggtattgatttaggataaatttcaatcattgttccaaatttcttagattgtgtcttgatatttttctcatatgctatctacttaacagtaacatttttgtttgttttgttgcttactaagcatttaatgtagagctatatgcatgttatatgcatgttataaatgtgttttgcacttgtttgaccaaattatgaaattagacacaaactgaactgtgaatcgtgagcctggaactcaacttagaacaattctagcctttttttctgagatttgtacctgtggcattttagattcgtgacagaccttcaaactaagaattgatgctgacaagggagaaatgtagactacccctctaccttaattttgtgcataaggtttttctagcaccaaaattaagatttgcatttgttatacttgtcaagtttgtgaaaagtaaatgtaaaaaataaaacactagatagttggatctactaaaagaaaaatgattgtcacgttaaaataattatgtgaacagttaaacatgattaacatgttatcattttttttttactaaggccttcccctaaatgtgcagcatgatactctctttgaaaccattctgtgtggtttgataaacagaacataacttcccagttgtctcatctgcctgtagaactagagttaggggacagggcatttgttaaggagaggattcatgttgcaggcagtgaaaaagactggtcagggttggcagcatctcattactgttggcaggatttcagaaatgctccctaaaacaggaccaatcagaatgttttttcttgacagttcatagattgtcagtgacaggtgctgtctgctttccccagaaggaggtccatttctaattgcttattgtcatatgacttaaagggcactgaactagggcaaaggatgtattttattttattttatttttttgagacagagtctcattcatagtaactgtttgatagaaagcactcttaaaggtagacaggatagaaagggggaaagaaggataaattatagcattctactttattttgaaaaatgggtagtactttcaaatattgaaattgtctaagcagaagttttgcagttttgcacacaaaagcattaaagttttcacagcagtgtgactgtacttaatgccacttcactgtacacttaaaacattattaaaatggtaaaatgtatgttacatgtatttaccacaataaaaaaaaacatttttaaaaggaagtagtaaagtgttgaccctattaaaaaggaggagggcagtattttgggatttttaaggaccttgaaattaactgatagtttgaaacatatagcagagaactgataatctttttttaggtcatgaaagtaaaatgtttaagatacaatatttttggtctttttagtaaaggcatttgttttcagtaaagatacttcttttttaaaggagagaatttaggattaccatttggtaagagagtatatggaacaagagatattaataagagaagtagagtaatggaaagatctgaaactggtattgagctgtctcactccgttgcccaggctagggtgaagtggcatgatctcggctcactgcaacctctgcctcctgggctcaggctgggactacagtcacgtgccatcatgcctggctaattttttgtattttttgtagagatggggttttgccatgttgcgcaggctggtcttgagctcctggactcaagggatccacccgccttggcctcgtaacatgctgggattacaggcatgagccactgcgcctggcagaactggttaaaatatttagggactggaaaagcaaggggaaaggaggaaagaagttgctctagagtagggctgtggcaagcagtaaatggtattgtttttgatacattgaacactgctttccaaagtgaatattttctatttgatgagctaattttaaagtaattattttttcttgcttaccaggaattttctacttctggtcatgcttacactgatacagggaaagcatcaggcaacctagaaaccaaatataaggtctgtaactatggacttaccttcacccagaaatggaacacagacaatactctagggacagaaatctcttgggagaataagttggctgaagggttgaaactgactcttgataccatatttgtaccgaacacaggaaagaagagtgggaaattgaaggcctcctataaacgggattgttttagtgttggcagtaatgttgatatagatttttctggaccaaccatctatggctgggctgtgttggccttcgaagggtggcttgctggctatcagatgagttttgacacagccaaatccaaactgtcacagaataatttcgccctgggttacaaggctgcggacttccagctgcacacacatgtgaacgatggcactgaatttggaggttctatctaccagaaggtgaatgagaagattgaaacatccataaaccttgcttggacagctgggagtaacaacacccgttttggcattgctgctaagtacatgctggattgtagaacttctctctct

SEQ ID NO: 152 - RNA, 585 nt, unidentified
  source 1..585: mol_type = other RNA
gaattttctacttctggtcatgcttacactgatacagggaaagcatcaggcaacctagaaaccaaatataaggtctgtaactatggacttaccttcacccagaaatggaacacagacaatactctagggacagaaatctcttgggagaataagttggctgaagggttgaaactgactcttgataccatatttgtaccgaacacaggaaagaagagtgggaaattgaaggcctcctataaacgggattgttttagtgttggcagtaatgttgatatagatttttctggaccaaccatctatggctgggctgtgttggccttcgaagggtggcttgctggctatcagatgagttttgacacagccaaatccaaactgtcacagaataatttcgccctgggttacaaggctgcggacttccagctgcacacacatgtgaacgatggcactgaatttggaggttctatctaccagaaggtgaatgagaagattgaaacatccataaaccttgcttggacagctgggagtaacaacacccgttttggcattgctgctaagtacatgctggattgtagaacttctctctct

SEQ ID NO: 153 - RNA, 1177 nt, unidentified
  source 1..1177: mol_type = other RNA
atattttagggttgccgttcttccttcctcaactgatgtcagctgtctgtttcgcacaaaagttcatccgcccacagaatccattttattcaatgatgtgttcagagtcgccatttcccaaacagccttacaacagaagacactgagggtagacctttgctctgtcagtaaacaccgaagggaagaatgcctggctggaactcagatcagcctggcagatttaccattttccagtgaggttttcactctatggtataacttgcttccttccaagcaaatgccttgcaaaaagaatgaagaaaatgaggactctgtatttcaaccaaaccagccgttagtagattctatagacttggatgcagtgtcagccttacttgcaagaacatcagctgagttgttagctgtggaacaagaattagcacaagaagaagaagaagaatcaggacaagaagagccaaggggcccagatggagactggctaacaatgctaagagaggcctctgatgaaattgtggctgaaaaagaggctgaagttaaattgccagaggacagtagctgtacagaagatttaagttcatgcactagtgtgcctgagatgaatgaagacgggaacaggaaagaaagcaactgtgccaaagacctcagaagtcagccacctactagaataccaacactggttgacaaagagacaaacactgatgaagccgctaatgacaatatggcagttcgccccaaagagcgcagcagcctgagctctagacagcatccgtttgtgaggagcagtgtgatagtgcgctcacagaccttttctccaggagagcggaaccagtacatctgcaggttaaatcggagtgacagtgacagttcaaccctggctaaaaaatcactgtttgtgagaaactccaccgaacgccgcagtttgagggtcaaaaggacggtttgccagtcagtccttagaagaacaacacaggaatgcccagtgcggacatctctagacttagaactggaccttcaggcatctctgacccggcagagccgcctcaatgatgagctgcaggcgctgagggacttgcggcagaagctggaggaactgaaagctcagggagagactgaccttccaccaggcgtgctggaggatgagaggttccagaggcttctgaagcaagctgagaagcag

SEQ ID NO: 154 - RNA, 2260 nt, unidentified
  source 1..2260: mol_type = other RNA
gcgctcagggaagcaacttcctctccactctgaaggagacaccagcaagcgtgctggagttggtgatctgcttcttctccatctggtccattctgggcctctcagggtttcacacgtacctcgtcgcctccaacctgactactaatgaagacatcaaaggctcgtggtccagcaagaggggcggtgaggcctctgtcaacccctacagccataaaagtattatcaccaactgctgtgctgtgctctgtggccccctacctcccagcctaattgaccggaggggatttgtgcagtccgacaccgtgttgccctcacccatcagaagcgatgagccagcctgcagagccaagcctgatgccagcatggtaggaggccacccctgaccacggctcagtacttgccacctgctggcctgtctgaccctccgcactcacctgccgggaccctccctattccatccaagggaagcagaactgccaaagactcaagtcttttcatatttatttcccatcctgcgtggctttccctgaactgttccgtggctgtgccctctgctccccaaacccaggttcccacagccttgggccctaggtaccccagctgatcagtgccaggagagaccagagcctctggaggctacccaggggaccacaccaagtccttgcctgtgccgggcgagccctgtgtgagtgaggctgtgaactgagcgtgaggcctcccaggtgggggaactgcttgggccttgctgagccagggtcctcagggtgaagcaggactgaggagtggccagctctggatagctggctgtggagaggaagcctccatgggctgctttggtctgtgggctccttcattcccttggtgataatttccctttattctgtgggatttttggtggggttttccccccttttttatggagttggccaataggattgagttggggctccagtagagaaggcagggttggtggtgggtgggggcagcctgtatcagacaaaggtaaatcagccagccaggcacccacagcctcagctcctgtgcagttcctgggcagcacagtggaagtgggagcctggtccttcccctgcccatggagagctctttaagggatcccagcctgcccctccacttctctcccaagccaggtcccggcatgggtgggttatgctcatgctggcaatacttgaaacgggtttattaatgctgggtattttgcacaattttatagacctcttttctacatagtcttttttaaatggaaggagaaaatgtcagccacattactgtctgtgtagtgccaggtgaagggttatcagaaggctggttggttttaataagtttattccaagagaccttctggctggaatgagtgagagtgtgtgtgcatgtgtgtgtgtgttcatgtgtgccctgtatgaatgtggctggctcccatatcccctgggctgccccctgccccatcccctttgagtgtcagaagcactctgagccaaggggacagggggcacgtgcactggtcacgagaaaaccctgggctcccactggggctcagcccagcctcctatctttccttcttctatggacttcagacagccagtgtctggggactctgccactctacccccagccctacccaccagcccccaggtgaggcttccagctgggacctgcccagacaggctgagcctgggcgtggtgggtggggtgatggctctggggagcggctgccatcctacaagccacaccccctcctctgagctctgaatatgggacccagtgccaggagctggaagacaaggtgtttctgccaaacggggacctccatccagagaaaaggaagaaggtgcagggtgggccaagaggcaagtgaaggttggcctgagtctgggccggaaactcagaggatgtttctcctctgctgggagctgtagtttcttatcaaaatagatattgttccaccatccccctccttggcccttcaagtgggctgaagcccttggaaagtgacataggaagtccccagatcttgcccttctcactccagaggctagtggtcacagacagctgggaatggcagccacagagggtcccctctgggagaaacagcttcaccccagcctcagggccctgggccatcactgcagtggccctgggaggtgaggaagaagctggctagaggagggggctcccacctaccttttatttaagccagtattctttgttcctgcttgtaataaaacttcagtttataagagtt

SEQ ID NO: 155 - DNA, 16 bp, unidentified
  source 1..16: mol_type = other DNA
ccgataagacgaacaa

SEQ ID NO: 156 - DNA, 15 bp, unidentified
  source 1..15: mol_type = other DNA
gagcaggctggagaa

SEQ ID NO: 157 - DNA, 22 bp, unidentified
  source 1..22: mol_type = other DNA
ataagacgaacaaaaggtttgt

SEQ ID NO: 158 - DNA, 20 bp, unidentified
  source 1..20: mol_type = other DNA
catcgtcggtgaagagggac

SEQ ID NO: 159 - DNA, 20 bp, unidentified
  source 1..20: mol_type = other DNA
tgagtttgtgactccggtgg

SEQ ID NO: 160 - DNA, 60 bp, unidentified
  source 1..60: mol_type = other DNA
acaatctccctgtggacttgaaggagatcttcactctgtaatcaacaccttccttcagga

SEQ ID NO: 161 - DNA, 20 bp, unidentified
  source 1..20: mol_type = other DNA
gtcccaccttctgggacaaa

SEQ ID NO: 162 - DNA, 20 bp, unidentified
  source 1..20: mol_type = other DNA
gacactgggatgagtggagc

SEQ ID NO: 163 - DNA, 60 bp, unidentified
  source 1..60: mol_type = other DNA
attgaagttttagtggggtacgagccagagcagagaaatcttcctctctctagcatctgt

SEQ ID NO: 164 - DNA, 20 bp, unidentified
  source 1..20: mol_type = other DNA
ggccaactctctctgagctg

SEQ ID NO: 165 - DNA, 20 bp, unidentified
  source 1..20: mol_type = other DNA
gaggtatcctccatgcgtcc

SEQ ID NO: 166 - DNA, 60 bp, unidentified
  source 1..60: mol_type = other DNA
ttgctgtctttgtcaaagtgaatgacaacaatttggccaactctctctgagctgcactca

SEQ ID NO: 167 - DNA, 20 bp, unidentified
  source 1..20: mol_type = other DNA
accttctttggtgcaaccct

SEQ ID NO: 168 - DNA, 20 bp, unidentified
  source 1..20: mol_type = other DNA
tgctccactatgtgcttgct

SEQ ID NO: 169 - DNA, 60 bp, unidentified
  source 1..60: mol_type = other DNA
ctttcattggggtccacatatagcctcagttacattagctgcttatgaatgcaattcagt

SEQ ID NO: 170 - DNA, 20 bp, unidentified
  source 1..20: mol_type = other DNA
gctgctactgggagatccac

SEQ ID NO: 171 - DNA, 20 bp, unidentified
  source 1..20: mol_type = other DNA
ggaggatgcggtggtagatg

SEQ ID NO: 172 - DNA, 60 bp, unidentified
  source 1..60: mol_type = other DNA
tggcatcacaggcagttcctctttgtggttgccttcactaccttcctggtcagctgcgtg

SEQ ID NO: 173 - DNA, 19 bp, unidentified
  source 1..19: mol_type = other DNA
cctttccttacgcacgctg

SEQ ID NO: 174 - DNA, 20 bp, unidentified
  source 1..20: mol_type = other DNA
ctgagggagggacttcagca

SEQ ID NO: 175 - DNA, 60 bp, unidentified
  source 1..60: mol_type = other DNA
aattgtattaaaattgatcatccacagaacttcctttccttacgcacgctgcgcgcggac

SEQ ID NO: 176 - DNA, 20 bp, unidentified
  source 1..20: mol_type = other DNA
cagcagctgccatctctcaa

SEQ ID NO: 177 - DNA, 20 bp, unidentified
  source 1..20: mol_type = other DNA
aacgcctgagcagctgattt

SEQ ID NO: 178 - DNA, 60 bp, unidentified
  source 1..60: mol_type = other DNA
ttttgtcaaggacctcagcaatggagaatatccccatcatcacaactgtcatcactctgc

SEQ ID NO: 179 - DNA, 20 bp, unidentified
  source 1..20: mol_type = other DNA
gatgtcaccaagtgcctgga

SEQ ID NO: 180 - DNA, 20 bp, unidentified
  source 1..20: mol_type = other DNA
gtcctctaggtcagccagga

SEQ ID NO: 181 - DNA, 60 bp, unidentified
  source 1..60: mol_type = other DNA
atcatcagcaaaatgatcattaatgaggagctgatgcttctctctcggcgtttccgctgt

SEQ ID NO: 182 - DNA, 20 bp, unidentified
  source 1..20: mol_type = other DNA
gcttcgggaagcagatagca

SEQ ID NO: 183 - DNA, 20 bp, unidentified
  source 1..20: mol_type = other DNA
aagcccctctggccttttag

SEQ ID NO: 184 - DNA, 60 bp, unidentified
  source 1..60: mol_type = other DNA
tttataagaagtggcgacagacaaagttgcagaaaagctgagctctactctctcatgggt

(19) United States
(12) Patent Application Publication
(10) Pub. No.: US 2025/0115962 A1
(43) Pub. Date: Apr. 10, 2025

(54) METHOD FOR PREDICTING ACUTE MYOCARDIAL INFARCTION

(71) Applicant: The First Affiliated Hospital of Ningbo University, Ningbo City (CN)

(72) Inventors: Hanbin CUI, Ningbo City (CN); Pengpeng SU, Ningbo City (CN); Teng HU, Ningbo City (CN); Ning HUANGFU, Ningbo City (CN); Jiajun YING, Ningbo City (CN); Jiaxi SHEN, Ningbo City (CN); Jinsong CHENG, Ningbo City (CN); Junsong LIU, Ningbo City (CN); Shiqi WANG, Ningbo City (CN); Honglin ZHOU, Ningbo City (CN); Hengyi MAO, Ningbo City (CN); Zhenwei LI, Ningbo City (CN)

(21) Appl. No.: 18/910,168

(22) Filed: Oct. 9, 2024

(30) Foreign Application Priority Data
Oct. 10, 2023 (CN) 2023113131296

(51) Int. Cl. C12Q 1/6883

(52) U.S. Cl. CPC C12Q 1/6883; C12Q 2600/158; C12Q 2600/178

(57) ABSTRACT

The present invention provides a method for predicting whether an individual has early myocardial infarction. The method includes: providing a blood sample of the individual; testing a marker in the blood sample to obtain the quantity of the marker; comparing the obtained number with a preset threshold, and judging whether the individual will suffer from myocardial infarction by the comparison, wherein the marker is any one or more nucleotide sequences of miRNA, lncRNA or circRNA.

### CROSS-REFERENCE TO RELATED APPLICATIONS

The present application claims priority of Chinese prior application No. 2023113131293 filed on Oct. 10, 2023; all the content of which is incorporated by reference as a part of the present invention.

### SEQUENCE LISTING

A sequence listing is being submitted with this application. This sequence listing is submitted as file name “METHOD FOR EARLY DIAGNOSIS OF ACUTE MYOCARDIAL INFARCTION” with a file size of 317 KB and a date of creation of Sep. 27, 2024. This document is hereby incorporated by reference in its entirety.

### BACKGROUND OF THE INVENTION

### Field of the Invention

The present invention relates to the field of early screening of myocardial infarction, and more specifically, to a marker for predicting acute myocardial infarction and use thereof, and in particular to a marker for diagnosing and conducting risk stratification of acute myocardial infarction in a hyperacute phase (<6 hours) and use thereof.

### Description of the Related Art

Acute myocardial infarction (AMI), also known as acute myocardic infarction, is myocardial necrosis caused by acute and persistent ischemia and hypoxia of the coronary arteries. According to the criteria of World Health Organization (WHO): AMI can be diagnosed if two of three indicators including typical chest pain, electrocardiogram changes and abnormal cardiac enzymes are met. With the discovery and understanding of new myocardial injury markers, the current diagnosis of AMI is mainly based on the criteria in the fourth edition of the “Universal Definition of Myocardial Infarction”, that is, cardiac troponin (cTn) is elevated and is higher than an upper limit of a normal value (at 99 percentile value of an upper limit of a reference value) at least once), and meanwhile there are clinical evidences of acute myocardial ischemia, including: (1) symptoms of acute myocardial ischemia; (2) new ischemic electrocardiogramanges; (3) newly emitted pathological Q waves; (4) imageological evidence of new loss of viable myocardium or regional wall motion abnormality; and (5) coronary thrombosis confirmed by coronary angiography or intraluminal imaging examination or autopsy. If the “5+1” criteria are met, AMI can be diagnosed.

AMI has two clinical manifestations: angina pectoris and analgesia. In China, asymptomatic AMI patients account for about 25% of total diseased population, and about 30% of cases have no typical angina pectoris manifestations. Electrocardiogram is generally used for diagnosis clinically, and the accuracy of electrocardiogram in diagnosing AMI is about 60% on average. ST segment elevation with diagnostic significance is often manifested atypical in many cases, and pathological Q waves often appear 6-8 hours after onset. However, current electrocardiogram lacks specificity for the diagnosis of non-ST-elevation AMI (NSTEMI), so that the electrocardiogram diagnosis rate of early AMI is very low. However, 1-6 hours after the onset of early AMI is the prime time for thrombolytic therapy and interventional surgery, so that rapid diagnosis of early myocardial infarction within 6 hours of onset is a key step in determining treatment (JACC 2021,78:2218, https://www.ncbi.nlm.nih.gov/pubmed/34756652).

Exosomes, with a diameter of about 30-150 nm and a density of 1.13-1.21 g/ml, are membrane vesicles actively secreted by cells and are formed through a series of regulatory processes such as “endocytosis-fusion-excretion”. The exosomes occur naturally in body fluid, including blood, saliva, urine, ascites, and breast milk. The exosomes carry unique or key functional molecules of source cells, resulting in different biological functions of exosomes from different sources. Therefore, it is possible to determine the changes in certain proteins and nucleic acids in certain cells according to the determined substances contained in the exosomes. Currently, there are many diseases, including certain tumors, for which the exosomes in the plasma of a patient can be detected. The protein or nucleic acid content in a sample is studied through certain experimental and data analysis methods, and is compared with the sample data of normal individuals to achieve early diagnosis, providing an important basis for judging the therapeutic effect and prognosis.

AMI is characterized by high morbidity and high case-fatality rate, etc. The risk of sudden death is highest within a few hours after onset, which is also the golden window period for medical treatment. However, acute myocardial infarction (AMI) may lack typical clinical symptoms in the early stages of the disease. There is an urgent need to find exosome biomarkers that can be used for early diagnosis and disease evaluation of patients.

### BRIEF SUMMARY OF THE INVENTION

In view of the problems existed in the prior art, the present invention provides a marker for predicting a hyperacute phase of acute myocardial infarction and use thereof. By analyzing the transcriptome data of exosomes in the plasma of patients with hyperacute myocardial infarction and normal people, a series of novel miRNA, lncRNA, and circRNA markers that can efficiently distinguish patients with the hyperacute phase of acute myocardial infarction and normal people are found, and a competitive endogenous RNA regulatory relationship network related to the hyperacute phase of myocardial infarction is constructed. It can be used for efficient detection of the hyperacute phase of myocardial infarction, and can achieve sensitive and specific diagnosis of the hyperacute phase within 6 hours of onset, accurately diagnose and evaluate patients with acute myocardial infarction and potential high-risk population, so as to improve the early diagnosis rate of acute myocardial infarction, guide the implementation of precision medicine strategies, and thus improve the level of treatment for acute myocardial infarction.

The patient with “myocardial infarction in the hyperacute phase” described in the present invention are a group of population with similar characteristics, including: the hyperacute phase of acute myocardial infarction (within 6 hours after onset), acute chest pain, an acute coronary syndrome and the like disorders. According to the method provided by the present invention, it is mainly used for timely diagnosis of a patient who is considered to be with acute myocardial infarction (in the hyperacute phase), and meanwhile for identifying cardiogenic and non-cardiogenic causes of a patient with acute chest pain, so as to start a precision medical strategy as early as possible.

In an aspect, the present invention provides a method for predicting whether an individual has early myocardial infarction. The method includes providing an exosome of an individual, testing the content or quantity of a marker in the exosome, and judging whether the individual has a risk of myocardial infarction according to the quantity, wherein the marker is any one or more selected from miRNA, lncRNA or circRNA, and the miRNA is any one or more selected from the table below:

| Name | Sequence |
|---|---|
| hsa-miR-1307-3p_R + 1 | Seq ID NO. 1 |
| hsa-miR-143-3p_R + 1 | Seq ID NO. 2 |
| hsa-miR-27b-3p | Seq ID NO. 3 |
| hsa-miR-152-3p | Seq ID NO. 4 |
| hsa-miR-24-3p_R − 2 | Seq ID NO. 5 |
| hsa-miR-378a-3p | Seq ID NO. 6 |
| hsa-miR-499a-5p | Seq ID NO. 7 |
| hsa-let-7c-5p | Seq ID NO. 8 |
| hsa-miR-208b-3p | Seq ID NO. 9 |
| hsa-miR-584-5p_R − 1 | Seq ID NO. 10 |
| hsa-miR-1-3p | Seq ID NO. 11 |
| hsa-miR-125b-5p | Seq ID NO. 12 |
| hsa-miR-30a-3p_R − 1 | Seq ID NO. 13 |
| hsa-miR-145-3p_L − 2R + 1 | Seq ID NO. 14 |
| hsa-miR-320a-3p | Seq ID NO. 15 |
| hsa-miR-133a-3p_L − 1R + 1 | Seq ID NO. 16 |
| hsa-miR-126-5p | Seq ID NO. 17 |
| hsa-miR-423-5p | Seq ID NO. 18 |
| hsa-miR-378c_R − 5 | Seq ID NO. 19 |
| hsa-miR-744-5p_R − 1 | Seq ID NO. 20 |
| hsa-miR-574-5p_R − 2 | Seq ID NO. 21 |
| hsa-miR-877-5p_R + 2 | Seq ID NO. 22 |
| hsa-miR-1908-5p_R − 1 | Seq ID NO. 23 |
| hsa-miR-490-3p_R + 1 | Seq ID NO. 24 |
| hsa-miR-574-5p_R − 2 | Seq ID NO. 25 |
| hsa-miR-877-5p_R + 2 | Seq ID NO. 26 |
| hsa-miR-941 | Seq ID NO. 27 |
| hsa-miR-9983-3p | Seq ID NO. 28 |
| hsa-miR-375-3p | Seq ID NO. 29 |
| hsa-miR-342-3p | Seq ID NO. 30 |
| hsa-miR-150-5p | Seq ID NO. 31 |
| hsa-miR-194-5p_R − 1 | Seq ID NO. 32 |
| hsa-miR-660-5p | Seq ID NO. 33 |
| hsa-miR-181a-5p_R − 2 | Seq ID NO. 34 |
| hsa-miR-30c-5p_R + 1 | Seq ID NO. 35 |
| hsa-miR-215-5p_R − 1 | Seq ID NO. 36 |
| hsa-miR-200b-3p | Seq ID NO. 37 |

Further, the lncRNA is any one or more selected from the table below:

| Name | Sequence |
|---|---|
| lnc-DAAM1-2 | Seq ID NO. 38 |
| FTX | Seq ID NO. 39 |
| AC006130.3 | Seq ID NO. 40 |
| CALML3-AS1 | Seq ID NO. 41 |
| CTC-297N7.7 | Seq ID NO. 42 |
| DAB1-AS1 | Seq ID NO. 43 |
| DLEU1 | Seq ID NO. 44 |
| FOXG1-AS1 | Seq ID NO. 45 |
| KCNQ5-IT1 | Seq ID NO. 46 |
| LINC00493 | Seq ID NO. 47 |
| LINC00593 | Seq ID NO. 48 |
| LINC01136 | Seq ID NO. 49 |
| LINC01255 | Seq ID NO. 50 |
| LINC01559 | Seq ID NO. 51 |
| linc-TBX3-5 | Seq ID NO. 52 |
| lnc-AXIN1-1 | Seq ID NO. 53 |
| lnc-B9D1-1 | Seq ID NO. 54 |
| lnc-C12orf42-3 | Seq ID NO. 55 |
| lnc-CDC7-1 | Seq ID NO. 56 |
| lnc-CEPT1-1 | Seq ID NO. 57 |
| lnc-CLDN20-3 | Seq ID NO. 58 |
| lnc-COIL − 2 | Seq ID NO. 59 |
| lnc-DLG4-2 | Seq ID NO. 60 |
| lnc-EIF2AK4-3 | Seq ID NO. 61 |
| lnc-EPHX2-3 | Seq ID NO. 62 |
| lnc-FAM82A2-1 | Seq ID NO. 63 |
| lnc-FOXA2-3 | Seq ID NO. 64 |
| lnc-FOXJ1-2 | Seq ID NO. 65 |
| lnc-KIAA1467-1 | Seq ID NO. 66 |
| lnc-LILRB1-2 | Seq ID NO. 67 |
| lnc-MARCH3-5 | Seq ID NO. 68 |
| lnc-MIS12-4 | Seq ID NO. 69 |
| lnc-OIT3-2 | Seq ID NO. 70 |
| lnc-RAB36-4 | Seq ID NO. 71 |
| lnc-RGL4-2 | Seq ID NO. 72 |
| lnc-SLC17A7-1 | Seq ID NO. 73 |
| lnc-SMC1B-1 | Seq ID NO. 74 |
| lnc-STS-1 | Seq ID NO. 75 |
| lnc-TAAR6-1 | Seq ID NO. 76 |
| lnc-TBCCD1-5 | Seq ID NO. 77 |
| lnc-TCEAL4-1 | Seq ID NO. 78 |
| lnc-TMEM71-3 | Seq ID NO. 79 |
| lnc-TUBA1C-1 | Seq ID NO. 80 |
| lnc-ZDHHC9-1 | Seq ID NO. 81 |
| LOC100505715 | Seq ID NO. 82 |
| LOC100507073 | Seq ID NO. 83 |
| LOC102031319 | Seq ID NO. 84 |
| LOC102724651 | Seq ID NO. 85 |
| LOC153684 | Seq ID NO. 86 |
| LOC338694 | Seq ID NO. 87 |
| RP11-290F20.3 | Seq ID NO. 88 |
| RP11-366H4.1 | Seq ID NO. 89 |
| RP11-383M4.6 | Seq ID NO. 90 |
| RP11-415F23.4 | Seq ID NO. 91 |
| RP5-833A20.1 | Seq ID NO. 92 |
| RPL23AP32 | Seq ID NO. 93 |
| TMEM147-AS1 | Seq ID NO. 94 |
| XLOC_l2_001089 | Seq ID NO. 95 |
| XLOC_l2_006640 | Seq ID NO. 96 |
| XLOC_l2_007731 | Seq ID NO. 97 |
| XLOC_l2_009804 | Seq ID NO. 98 |

Further, the circRNA is any one or more selected from the table below:

| Name | Sequence |
|---|---|
| hsa_circ_0044880 | Seq ID NO. 99 |
| hsa_circ_0032837 | Seq ID NO. 100 |
| hsa_circ_0063420 | Seq ID NO. 101 |
| hsa_circ_0046151 | Seq ID NO. 102 |
| hsa_circ_0037779 | Seq ID NO. 103 |
| hsa_circ_0037777 | Seq ID NO. 104 |
| hsa_circ_0058356 | Seq ID NO. 105 |
| hsa_circ_0074657 | Seq ID NO. 106 |
| hsa_circ_0092053 | Seq ID NO. 107 |
| hsa_circ_0049892 | Seq ID NO. 108 |
| hsa_circ_0057694 | Seq ID NO. 109 |
| hsa_circ_0071517 | Seq ID NO. 110 |
| hsa_circ_0072207 | Seq ID NO. 111 |
| hsa_circ_0045961 | Seq ID NO. 112 |
| hsa_circ_0089955 | Seq ID NO. 113 |
| hsa_circ_0024057 | Seq ID NO. 114 |
| hsa_circ_0002512 | Seq ID NO. 115 |
| hsa_circ_0038850 | Seq ID NO. 116 |
| hsa_circ_0012424 | Seq ID NO. 117 |
| hsa_circ_0013160 | Seq ID NO. 118 |
| hsa_circ_0020080 | Seq ID NO. 119 |
| hsa_circ_0020079 | Seq ID NO. 120 |
| hsa_circ_0041015 | Seq ID NO. 121 |
| hsa_circ_0041019 | Seq ID NO. 122 |
| hsa_circ_0055058 | Seq ID NO. 123 |
| hsa_circ_0027427 | Seq ID NO. 124 |
| hsa_circ_0027430 | Seq ID NO. 125 |
| hsa_circ_0072550 | Seq ID NO. 126 |
| hsa_circ_0008774 | Seq ID NO. 127 |
| hsa_circ_0064438 | Seq ID NO. 128 |
| hsa_circ_0007281 | Seq ID NO. 129 |
| hsa_circ_0069099 | Seq ID NO. 130 |
| hsa_circ_0066990 | Seq ID NO. 131 |
| hsa_circ_0049010 | Seq ID NO. 132 |
| hsa_circ_0051307 | Seq ID NO. 133 |
| hsa_circ_0051309 | Seq ID NO. 134 |
| hsa_circ_0040608 | Seq ID NO. 135 |
| hsa_circ_0075505 | Seq ID NO. 136 |
| hsa_circ_0089252 | Seq ID NO. 137 |
| hsa_circ_0089254 | Seq ID NO. 138 |
| hsa_circ_0043926 | Seq ID NO. 139 |
| hsa_circ_0060956 | Seq ID NO. 140 |
| hsa_circ_0030255 | Seq ID NO. 141 |
| hsa_circ_0054031 | Seq ID NO. 142 |
| hsa_circ_0054028 | Seq ID NO. 143 |
| hsa_circ_0024781 | Seq ID NO. 144 |
| hsa_circ_0020846 | Seq ID NO. 145 |
| hsa_circ_0028008 | Seq ID NO. 146 |
| hsa_circ_0016676 | Seq ID NO. 147 |
| hsa_circ_0016674 | Seq ID NO. 148 |
| hsa_circ_0026141 | Seq ID NO. 149 |
| hsa_circ_0058248 | Seq ID NO. 150 |
| hsa_circ_0084117 | Seq ID NO. 151 |
| hsa_circ_0084119 | Seq ID NO. 152 |
| hsa_circ_0071500 | Seq ID NO. 153 |
| hsa_circ_0011024 | Seq ID NO. 154 |

In the present invention, by conducting high-depth whole transcriptome sequencing on the exosomes in the plasma of patients with early myocardial infarction and normal population, it has been found that in the miRNA, 28 genes are significantly upregulated, and 9 genes are significantly downregulated; in the lncRNA, 1 gene is significantly upregulated, and 60 genes are significantly downregulated; in the circRNA, 3 genes are significantly upregulated, and 53 genes are significantly downregulated, thereby screening out a series of miRNA, lncRNA, and circRNA markers that distinguish the patients with early myocardial infarction from the normal population. Then further by verifying through a large number of plasma samples from patients with myocardial infarction and normal population, 154 target sequences that are found to be abnormally upregulated or downregulated in patients with early myocardial infarction, are finally discovered and determined.

Further, the early myocardial infarction includes a hyperacute phase, and the hyperacute phase refers to a time window range of the patient after the early myocardial infarction occurs.

The time window range described in the present invention refers to the most effective golden time for thrombolytic therapy and interventional surgery after acute myocardial infarction, and is also a key link for effective treatment of acute myocardial infarction.

Further, the early myocardial infarction includes a hyperacute phase, and the hyperacute phase means that the patient will progress to acute myocardial infarction within 6 hours or less.

Further, the reagent is used for detecting the degree or amplitude of change in the content or quantity of a marker in a blood sample.

In some embodiments, the change in the content or quantity of the marker refers to upregulation or downregulation of the amount of gene expression.

The presence or absence or content of the marker here is a relative concept. For example, compared with the non-diseased group, in the diseased group, the expression amount of these specific genes is compared based on that of the diseased group or the non-diseased group as a benchmark. It may be that the expression amount of certain genes in the diseased group is higher than that in the non-diseased group. This high level has a statistical difference, such as a significant or extremely significant increase. Therefore, when judgment is made about these gene markers, if a gene marker is a single gene marker, and if the expression amount of the marker changes when the gene of a certain risk occurs, the change here may be a relative increase or a relative decrease. The difference in this relative increase or relative decrease has a significant difference, and of course, it may also be an extremely significant difference. Therefore, no matter what means is used for detection, a predetermined value can be used as a standard (cut-off value). For example, if the expression amount is increased by several times, being higher than this value is considered as that the content has changed. Having such a result can be used as a prediction or diagnostic value.

Further, the marker is a combination of any two or more nucleotide sequences selected from the sequence listing Seq ID NO. 1-Seq ID NO. 154.

The combination of the markers, can be a combination of any nucleotide sequences selected from Seq ID NO. 1-Seq ID NO. 154. Regardless of whether the combination of sequences all belongs to one of miRNA, lncRNA or circRNA, or some of the combination of sequences belong to miRNA, some belong to lncRNA or some belong to circRNA, employing this marker combination to predict early myocardial infarction can further improve the AUC value and improve the sensitivity and specificity of early myocardial infarction screening.

Further, the reagent is used for detecting the exosome in blood.

In some embodiments, the method of the present invention is suitable for analyzing low-concentration exosomes found in a sample of a mixed state, e.g. blood, feces or tissues, and preferably exosomes in blood samples.

In another aspect, the present invention provides a kit for detecting whether an individual is in a hyperacute phase of acute myocardial infarction, the kit including a detection reagent for the detection marker as described above.

In some embodiments, the detection reagent includes an amplification primer probe set for detecting the expression amount of a gene. The primer probe set can be designed according to a partial sequence selected from a gene sequence to be tested, and the expression amount of the gene to be tested can be detected after amplification by qPCR, ddPCR, RAA, etc.

In a further aspect, the present invention provides a marker combination for predicting whether an individual is in a hyperacute phase of acute myocardial infarction, wherein the marker combination is a combination of any two or more nucleotide sequences selected from the sequence list Seq ID NO. 1-Seq ID NO. 154.

In yet a further aspect, the present invention provides a system for predicting whether an individual is in a hyperacute phase of acute myocardial infarction. The system including a data analysis module. The data analysis module is used for analyzing a detection value of a marker, wherein the marker is any one or more nucleotide sequences selected from the sequence list Seq ID NO. 1-Seq ID NO. 154.

Further, the system further includes a data storage module, a data input interface and a data output interface. The data storage module is used for storing a detection value of a biomarker. The data input interface is used for inputting the detection value of the biomarker. The data output interface is used for outputting a prediction result.

In still yet a further aspect, the present invention provides a method for screening a marker for predicting whether an individual is in a hyperacute phase of acute myocardial infarction, the method being based on whole transcriptome sequencing and including the following steps:
(1) conducting whole transcriptome sequencing of exosomes in the plasma of a patient in a hyperacute phase of acute myocardial infarction and normal people to obtain expression level data of mRNAs, miRNAs, lncRNAs, and circRNAs;
(2) predicting binding sites of the mRNAs, lncRNAs, circRNAs and miRNAs according to sequences obtained by the whole transcriptome sequencing of the exosomes;
(3) screening for mRNAs that are differentially expressed in the plasma of the patient in the hyperacute phase of acute myocardial infarction and normal people, according to the predicted mRNAs that have regulatory relationships with the miRNAs;
(4) conducting pathway enrichment analysis on the differentially expressed mRNAs regulated by the miRNAs to screen for significant pathways, screening for pathways related to myocardial infarction according to the enriched significant pathways, obtaining differentially expressed mRNAs involved in the pathways, and screening for differential miRNAs that have a regulatory relationship with the mRNAs; and
(5) screening for differential lncRNAs and circRNAs that have regulatory relationships with the miRNAs according to the miRNAs obtained as described above, so as to construct a competitive endogenous RNA regulatory relationship network related to the hyperacute phase of acute myocardial infarction.

Further, the method for predicting a marker of the hyperacute phase of acute myocardial infarction by constructing a regulatory network based on whole transcriptome sequencing includes the following steps:
(1) acquiring plasma from a group of patients in the hyperacute phase of early acute myocardial infarction and a normal control group, extracting exosomes from the plasma by an ultracentrifugation method, and conducting whole transcriptome sequencing of the exosomes to obtain expression level data of mRNAs, miRNAs, lncRNAs, and circRNAs;
(2) predicting binding sites of the mRNAs, lncRNAs, circRNAs and miRNAs according to the sequences obtained by the whole transcriptome sequencing of the exosomes;
(3) screening for mRNAs that are differentially expressed in exosomes derived from the plasma of a group of patients in the hyperacute phase of acute myocardial infarction and a normal control group, according to the predicted mRNAs that have regulatory relationships with the miRNAs;
(4) conducting pathway enrichment analysis on the differentially expressed mRNAs regulated by the miRNAs to screen for a significant pathway, screening for a pathway related to the hyperacute phase of acute myocardial infarction according to the enriched significant pathway, obtaining differentially expressed mRNAs involved in the pathway, and screening for differential miRNAs that have regulatory relationships with the mRNAs; and
(5) screening for differential lncRNAs and circRNAs that are predicted to have regulatory relationships with the miRNAs according to the miRNAs obtained as described above, so as to construct a competitive endogenous RNA regulatory relationship network related to the hyperacute phase of acute myocardial infarction.

In some embodiments, the expression level data of the mRNAs, miRNAs, lncRNAs, and circRNAs in step (1) are subjected to normalization treatment.

Further, in the step (2), the binding sites of the mRNAs, lncRNAs, and circRNAs with the miRNAs obtained by whole transcriptome sequencing are respectively predicted by employing prediction software TargetScan and/or miRanda with the parameters being set respectively. Thus the predicted miRNAs binding sites are obtained respectively, and the intersection of the analysis results of the two software is taken to obtain a final result of the regulatory relationship of the differentially expressed mRNAs, lncRNAs and circRNAs with the miRNAs.

In some embodiments, the screening parameters for predicting the mRNAs regulated by the miRNAs are removing a target gene with a context score percentile less than 80 in a TargetScan algorithm and removing a target gene with a maximum free energy (Max Energy) greater than-20 in a miRanda algorithm.

In some embodiments, the screening parameters for screening for mRNAs, miRNAs, lncRNAs, and circRNAs that are differentially expressed in the exosomes derived from the plasma of the group of patients in the hyperacute phase of acute myocardial infarction and the normal control group are a difference fold Log 2FC and a P-value.

In some embodiments, the screening parameters corresponding to the differential mRNAs in the step (3) are that the absolute value of Log 2FC is greater than 1.5 and the P-value is less than 0.05.

In some embodiments, in the step (4), a pathway with an enriched gene count greater than or equal to 1.5 and a P-values less than 0.05 is considered significant, a pathway related to the hyperacute phase of acute myocardial infarction is screened out, differentially expressed mRNAs involved in the pathway are obtained, and differential miRNAs that have regulatory relationships with the mRNAs are screened out, wherein the corresponding screening parameters are miRNAs that have a P-value less than 0.05 and are abundantly expressed.

In some embodiments, in the step (5), the screening parameters for predicting the lncRNAs and circRNAs that have regulatory relationships with the differential miRNAs are to remove a relationship pair with a context score percentile less than 80 from the TargetScan algorithm and to remove a relationship pair with maximum free energy (Max Energy) greater than-20 from the miRanda algorithm.

In some embodiments, the screening parameters for screening the differential lncRNAs that have relationship pairs with the differential miRNAs are that the absolute value of Log 2FC is greater than 1.5 and the P-value is less than 0.05; and the screening parameters for the circRNAs are that the absolute value of Log 2FC is greater than 1.5 and the P-value is less than 0.05.

Further, the competitive endogenous RNA regulatory relationship network related to the hyperacute phase of acute myocardial infarction constructed by the aforementioned method includes:
(1) 28 significantly up-regulated miRNAs: hsa-miR-1307-3p_R+1, hsa-miR-143-3p_R+1, hsa-miR-27b-3p, hsa-miR-152-3p, hsa-miR-24-3p_R-2, hsa-miR-378a-3p, hsa-miR-499a-5p, hsa-let-7c-5p, hsa-miR-208b-3p, hsa-miR-584-5p_R-1, hsa-miR-1-3p, hsa-miR-125b-5p, hsa-miR-30a-3p_R-1, hsa-miR-145-3p_L-2R+1, hsa-miR-320a-3p, hsa-miR-133a-3p_L-1R+1, hsa-miR-126-5p, hsa-miR-423-5p, hsa-miR-378c_R-5, hsa-miR-744-5p_R-1, hsa-miR-574-5p_R-2, hsa-miR-877-5p_R+2, hsa-miR-1908-5p_R-1, hsa-miR-490-3p_R+1, hsa-miR-574-5p_R-2, hsa-miR-877-5p_R+2, hsa-miR-941, hsa-miR-9983-3p; and 9 significantly down-regulated miRNAs: hsa-miR-375-3p, hsa-miR-342-3p, hsa-miR-150-5p, hsa-miR-194-5p_R-1, hsa-miR-660-5p, hsa-miR-181a-5p_R-2, hsa-miR-30c-5p_R+1, hsa-miR-215-5p_R-1, hsa-miR-200b-3p;
(2) 1 significantly up-regulated lncRNAs: lnc-DAAM1-2; and 60 significantly down-regulated lncRNAs: FTX, AC006130.3, CALML3-AS1, CTC-297N7.7, DAB1-AS1, DLEU1, FOXG1-AS1, KCNQ5-IT1, LINC00493, LINC00593, LINC01136, LINC01255, LINC01559, linc-TBX3-5, lnc-AXIN1-1, lnc-B9D1-1, lnc-C12orf42-3, lnc-CDC7-1, lnc-CEPT1-1, lnc-CLDN20-3, lnc-COIL-2, lnc-DLG4-2, lnc-EIF2AK4-3, lnc-EPHX2-3, lnc-FAM82A2-1, lnc-FOXA2-3, lnc-FOXJ1-2, lnc-KIAA1467-1, lnc-LILRB1-2, lnc-MARCH3-5, lnc-MIS12-4, lnc-OIT3-2, lnc-RAB36-4, lnc-RGL4-2, lnc-SLC17A7-1, lnc-SMC1B-1, lnc-STS-1, lnc-TAAR6-1, lnc-TBCCD1-5, lnc-TCEAL4-1, lnc-TMEM71-3, lnc-TUBA1C-1, lnc-ZDHHC9-1, LOC100505715, LOC100507073, LOC102031319, LOC102724651, LOC153684, LOC338694, RP11-290F20.3, RP11-366H4.1, RP11-383M4.6, RP11-415F23.4, RP5-833A20.1, RPL23AP32, TMEM147-AS1, XLOC_12_001089, XLOC_12_006640, XLOC_12_007731, XLOC_12_009804; and
(3) 3 significantly up-regulated circRNAs: hsa_circ_0044880, hsa_circ_0032837, hsa_circ_0063420; and 53 down-regulated circRNAs: hsa_circ_0046151, hsa_circ_0037779, hsa_circ_0037777, hsa_circ_0058356, hsa_circ_0074657, hsa_circ_0092053, hsa_circ_0049892, hsa_circ_0057694, hsa_circ_0071517, hsa_circ_0072207, hsa_circ_0045961, hsa_circ_0089955, hsa_circ_0024057, hsa_circ_0002512, hsa_circ_0038850, hsa_circ_0012424, hsa_circ_0013160, hsa_circ_0020080, hsa_circ_0020079, hsa_circ_0041015, hsa_circ_0041019, hsa_circ_0055058, hsa_circ_0027427, hsa_circ_0027430, hsa_circ_0072550, hsa_circ_0008774, hsa_circ_0064438, hsa_circ_0007281, hsa_circ_0069099, hsa_circ_0066990, hsa_circ_0049010, hsa_circ_0051307, hsa_circ_0051309, hsa_circ_0040608, hsa_circ_0075505, hsa_circ_0089252, hsa_circ_0089254, hsa_circ_0043926, hsa_circ_0060956, hsa_circ_0030255, hsa_circ_0054031, hsa_circ_0054028, hsa_circ_0024781, hsa_circ_0020846, hsa_circ_0028008, hsa_circ_0016676, hsa_circ_0016674, hsa_circ_0026141, hsa_circ_0058248, hsa_circ_0084117, hsa_circ_0084119, hsa_circ_0071500, hsa_circ_0011024.

In still yet a further aspect, the present invention provides use of miRNAs, lncRNAs, and circRNAs in a competitive endogenous RNA regulatory relationship network as diagnostic markers for a hyperacute phase of acute myocardial infarction.

The method for screening a patient in a hyperacute phase of acute myocardial infarction provided by the present invention has the following beneficial effects:
1. 154 markers for novel screening of the hyperacute phase of acute myocardial infarction are provided, the expression status of these markers in the plasma of patients in the hyperacute phase of acute myocardial infarction and normal population is significantly different, and abnormal upregulation or downregulation will occur;
2. any one, two or more markers can be selected from the 154 markers of acute myocardial infarction and combined to detect the hyperacute phase of acute myocardial infarction with high sensitivity and specificity;
3. a competitive endogenous RNA regulatory relationship network related to the hyperacute phase of acute myocardial infarction is constructed;
4. it can realize sensitive and specific diagnosis of the hyperacute phase of acute myocardial infarction within 6 hours of onset;
5. the method is convenient and rapid, and the detection results are highly consistent with clinical gold standard test results; and
6. this method accurately locates the high-risk population of patients with acute myocardial infarction, and accurately evaluates the cardiovascular risk of this population, so as to facilitate early discovery and early intervention, promote early discovery and early treatment of acute myocardial infarction in the hyperacute phase, and meet the urgent clinical needs.

### DETAILED DESCRIPTION

### (1) Diagnosis or Detection

Here diagnosis or detection is predicted to refer to the detection or assay of a biomarker in a sample, or the content of a biomarker of interest, such as absolute content or relative content, and then whether the individual from which the sample is provided may have or suffer from a certain disease or have the possibility of a certain disease is illustrated through the presence or absence or quantity of the biomarker of interest. The meanings of diagnosis and detection here are interchangeable. The result of this detection or diagnosis cannot be directly regarded as a direct result of suffering from a disease, but an intermediate result. If a direct result is obtained, it is necessary to confirm suffering from a disease through other auxiliary means such as pathology or anatomy. For example, the present invention provides a variety of new biomarkers that are associated with the occurrence of early myocardial infarction, and changes in the content of these markers are directly related to whether the patient suffers from early myocardial infarction.

(2) Association of a Marker or Biomarker with Early Myocardial Infarction

The marker and biomarker have the same meaning in the present invention. The association here means that the presence or change in the content of a certain biomarker in a sample is directly related to a specific disease or the progress of the disease. For example, a relative increase or decrease in the content indicates the possibility of suffering from the disease is higher than that of healthy people, or indicates the progress of the disease develops more seriously or develops from one stage to another. For example, a single marker or a combination of marker substances of multiple new markers of the present invention can be used for predicting whether early myocardial infarction will occur.

If a number of different markers in the sample appear at the same time or the content changes relatively, it means that the possibility of suffering from this disease is higher than that of healthy people. That is, among the types of markers, some markers have strong association with suffering from a disease, some markers have weak association with suffering from a disease, or some even have no association with a specific disease. One or more of the markers with strong association can be used as markers for diagnosing a disease, and those markers with weak association can be combined with strong markers to diagnose a certain disease to increase the accuracy of detection results. The disease here can be the process or progress of the disease, for example, developed from a better stage of a disease to a more malignant or serious stage, or even finally death.

For the numerous biomarkers found in the serum of the present invention, these markers all can be used for determining whether the patient is a patient with early myocardial infarction; and they can also be used for diagnosing or predicting the probability or possibility of early myocardial infarction. The markers here can be used as individual markers for direct detection or diagnosis. Selecting such a marker indicates that the relative change in the content of the marker is strongly associated with a patient with early myocardial infarction. Of course, it can be understood that simultaneous detection of one or more markers for early diagnosis of acute myocardial infarction can be selected. The normal understanding is that in some embodiments, selecting a biomarker with strong association for detection or diagnosis can achieve a certain standard of accuracy, for example an accuracy of 60%, 65%, 70%, 80%, 85%, 90% or 95%. Then it can be explained that these markers can obtain an intermediate value for diagnosing a certain disease, but it does not mean that it can directly confirm suffering from a certain disease.

Of course, you can also choose a differential marker with a larger AUC value as a diagnostic marker. The so-called strong and weak are generally calculated and confirmed through some algorithms, for example the contribution rate or weight analysis of markers and the probability of early myocardial infarction. Such a calculation method can be significance analysis (a p value or FDR value) and fold change, and multivariate statistical analysis mainly including principal component analysis (PCA) and partial least squares-discriminant analysis (PLS-DA).

(3) Patients with Early Myocardial Infarction

The patients with early myocardial infarction refer to patients who may progress to acute myocardial necrosis due to persistent myocardial ischemia, but the onset time is short (less than 6 hours) and the clinical phenotype is atypical, and the currently commonly-used troponin detection cannot clearly diagnose the patients. These patients may progress to large-area myocardial infarction, or the infarct area may be relatively limited with timely treatment, i.e., small-scale AMI.

Therefore, the prediction method provided by the present invention can quickly identify patients with early myocardial infarction among patients with myocardial injury or unstable angina pectoris and small-scale AMI, so as to provide early intervention and treatment.

(4) Epidemiological features of acute myocardial infarction: patients with symptoms that may radiate to the left upper limb, such as acute chest pain, chest tightness or throat tightness, including patients with new chest pain or acute exacerbation of existing chest pain. Such patients are accompanied or not accompanied by clinical symptoms such as profuse sweating, fever, tachycardias, fatigue, dizziness, syncope, hypotension and shock.

Electrocardiogram: ST segment (elevation or depression) and T wave (flattening or inversion) changes, where dynamic changes in the ST segment (elevation or depression ≥0.1 mV) are characteristic manifestations of coronary artery lesions. There are also some patients whose electrocardiogram has no changes or has changes lacking specificity.

Laboratory tests: myocardial injury markers such as troponin, creatine kinase, myoglobin, etc. have not yet been increased with diagnostic significance; and they may be accompanied by an increase in white blood cells, an increase in C-reactive protein, an increase in the erythrocyte sedimentation rate, etc.

### BRIEF DESCRIPTION OF THE DRAWINGS

FIG. 1 is a flow chart of marker target sequence screening in Example 1; and

FIGS. 2 to 4 are ROC analysis diagrams of single marker target sequences in Example 2, wherein FIG. 2 is a ROC analysis diagram of a SEQ ID NO. 9 sequence,

FIG. 3 is a ROC analysis diagram of a SEQ ID NO. 83 sequence, and

FIG. 4 is a ROC analysis diagram of a SEQ ID NO. 118 sequence.

### DETAILED DESCRIPTION OF THE INVENTION

The present invention will be further described in detail with reference to accompanying drawings and examples, and it should be pointed out that the following examples are intended to facilitate the understanding of the present invention, without any limitation to it. The reagents used in the present examples are all known products and obtained by purchasing commercially available products.

### Example 1 Screening of Markers for Predicting a Hyperacute Phase of Acute Myocardial Infarction

In this example, plasma samples of clinical patients with early myocardial infarction (100 cases) and normal population (100 cases) were obtained, exosomes in plasma were extracted by an ultracentrifugation method, and complete transcriptome sequencing was conducted on the exosomes to obtain the expression level data of mRNAs, miRNAs, lncRNAs and circRNAs. The expression level data of the mRNAs, miRNAs, lncRNAs and circRNAs were subjected to normalization treatment, wherein the screening parameters are a differential fold Log 2FC and a P-value.

According to the sequences obtained by whole transcriptome sequencing of the exosomes, the binding sites of the mRNAs, lncRNAs, and circRNAs with the miRNAs were predicted: prediction software TargetScan and/or miRanda were employed with parameters being set respectively to obtain the predicted miRNAs binding sites respectively, and the intersection of the analysis results of the two software was taken to obtain a final result of a regulatory relationship between the differentially expressed mRNAs, lncRNAs, circRNAs and the miRNAs; wherein, the screening parameters for predicting the mRNAs regulated by the miRNAs were to remove a target gene with a context score percentile less than 80 from a TargetScan algorithm and to remove a target gene with maximum free energy (Max Energy) greater than-20 from a miRanda algorithm.

According to the predicted mRNAs with regulatory relationships with the miRNAs, the mRNAs that were differentially expressed in the plasma derived from the exosomes of a group of patients with early acute myocardial infarction and a normal control group were screened out; the screening parameters for the miRNAs, lncRNAs, and circRNAs were the difference fold Log 2FC and the P-value; and the screening parameters corresponding to the differential mRNAs were the absolute value of Log 2FC greater than 1.5 and the P-value less than 0.05.

Pathway enrichment analysis was conducted on the differentially expressed mRNAs regulated by the miRNAs to screen for a significant pathway. A pathway related to myocardial infarction was screened out according to the enriched significant pathway. Differentially expressed mRNAs involved in the pathway were obtained, and differential miRNAs with regulatory relationships therewith were screened out. A pathway with an enriched gene count greater than or equal to 2 and a P-value less than 0.05 is considered significant. A pathway related to myocardial infarction was screened out. Differentially expressed mRNAs involved in the pathway were obtained, and differential miRNAs with regulatory relationships therewith were screened out, wherein the corresponding screening parameters were an absolute value of Log 2FC greater than 2 and a P-value less than 0.05.

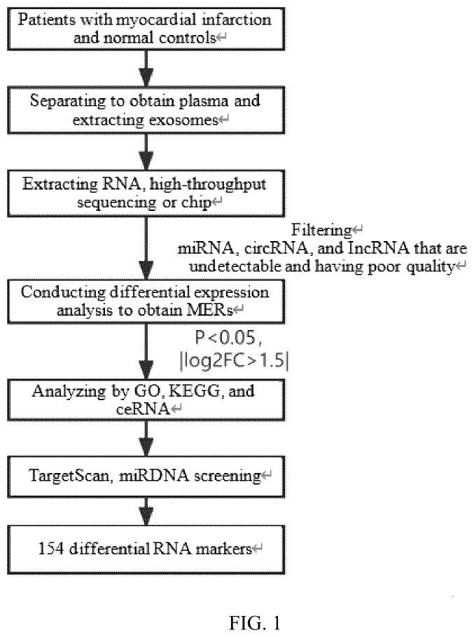

According to the miRNAs obtained as described above, differential lncRNAs and circRNAs that were predicted to have regulatory relationships with the miRNAs were screened out to construct a competitive endogenous RNA regulatory relationship network associated with early myocardial infarction. The screening parameters for predicting the lncRNAs and circRNAs that had regulatory relationships with the differential miRNAs were to remove a relationship pair with a context score percentile less than 80 from the TargetScan algorithm and to remove a relationship pairs with maximum free energy (Max Energy) greater than-20 from the miRanda algorithm. The screening parameters for screening for differential lncRNAs that had relationship pairs with differential miRNAs were an absolute value of Log 2FC greater than 1.5 and a P-value less than 0.05; and the screening parameters for the circRNAs were an absolute value of Log 2FC greater than 1.5 and a P-value less than 0.05 (see FIG. 1 for the screening flow chart).

Finally, 154 target sequence markers with abnormal up-regulated or down-regulated expression in patients with early myocardial infarction were found and determined, including 37 miRNA markers, 61 lncRNA markers and 56 circRNA markers, which were respectively:
(1) 28 significantly up-regulated miRNAs (Seq ID NO. 1-Seq ID NO. 28): hsa-miR-1307-3p_R+1, hsa-miR-143-3p_R+1, hsa-miR-27b-3p, hsa-miR-152-3p, hsa-miR-24-3p_R-2, hsa-miR-378a-3p, hsa-miR-499a-5p, hsa-let-7c-5p, hsa-miR-208b-3p, hsa-miR-584-5p_R-1, hsa-miR-1-3p, hsa-miR-125b-5p, hsa-miR-30a-3p_R-1, hsa-miR-145-3p_L-2R+1, hsa-miR-320a-3p, hsa-miR-133a-3p_L-1R+1, hsa-miR-126-5p, hsa-miR-423-5p, hsa-miR-378c_R-5, hsa-miR-744-5p_R-1, hsa-miR-574-5p_R-2, hsa-miR-877-5p_R+2, hsa-miR-1908-5p_R-1, hsa-miR-490-3p_R+1, hsa-miR-574-5p_R-2, hsa-miR-877-5p_R+2, hsa-miR-941, hsa-miR-9983-3p; and 9 significantly down-regulated miRNAs (Seq ID NO. 29-Seq ID NO. 37): hsa-miR-375-3p, hsa-miR-342-3p, hsa-miR-150-5p, hsa-miR-194-5p_R-1, hsa-miR-660-5p, hsa-miR-181a-5p_R-2, hsa-miR-30c-5p_R+1, hsa-miR-215-5p_R-1, hsa-miR-200b-3p;
(2) 1 significantly up-regulated lncRNA (Seq ID NO. 38): lnc-DAAM1-2; and 60 significantly down-regulated lncRNAs (Seq ID NO. 39-Seq ID NO. 98): FTX, AC006130.3, CALML3-AS1, CTC-297N7.7, DAB1-AS1, DLEU1, FOXG1-AS1, KCNQ5-IT1, LINC00493, LINC00593, LINC01136, LINC01255, LINC01559, linc-TBX3-5, lnc-AXIN1-1, lnc-B9D1-1, lnc-C12orf42-3, lnc-CDC7-1, lnc-CEPT1-1, lnc-CLDN20-3, lnc-COIL-2, lnc-DLG4-2, lnc-EIF2AK4-3, lnc-EPHX2-3, lnc-FAM82A2-1, lnc-FOXA2-3, lnc-FOXJ1-2, lnc-KIAA1467-1, lnc-LILRB1-2, lnc-MARCH3-5, lnc-MIS12-4, lnc-OIT3-2, lnc-RAB36-4, lnc-RGL4-2, lnc-SLC17A7-1, lnc-SMC1B-1, lnc-STS-1, lnc-TAAR6-1, lnc-TBCCD1-5, lnc-TCEAL4-1, lnc-TMEM71-3, lnc-TUBA1C-1, lnc-ZDHHC9-1, LOC100505715, LOC100507073, LOC102031319, LOC102724651, LOC153684, LOC338694, RP11-290F20.3, RP11-366H4.1, RP11-383M4.6, RP11-415F23.4, RP5-833A20.1, RPL23AP32, TMEM147-AS1, XLOC_12_001089, XLOC_12_006640, XLOC_12_007731, XLOC_12_009804;
(3) 3 significantly up-regulated circRNAs (Seq ID NO. 99-Seq ID NO. 101): hsa_circ_0044880, hsa_circ_0032837, hsa_circ_0063420; and 53 significantly down-regulated circRNAs (Seq ID NO. 102-Seq ID NO. 154): hsa_circ_0046151, hsa_circ_0037779, hsa_circ_0037777, hsa_circ_0058356, hsa_circ_0074657, hsa_circ_0092053, hsa_circ_0049892, hsa_circ_0057694, hsa_circ_0071517, hsa_circ_0072207, hsa_circ_0045961, hsa_circ_0089955, hsa_circ_0024057, hsa_circ_0002512, hsa_circ_0038850, hsa_circ_0012424, hsa_circ_0013160, hsa_circ_0020080, hsa_circ_0020079, hsa_circ_0041015, hsa_circ_0041019, hsa_circ_0055058, hsa_circ_0027427, hsa_circ_0027430, hsa_circ_0072550, hsa_circ_0008774, hsa_circ_0064438, hsa_circ_0007281, hsa_circ_0069099, hsa_circ_0066990, hsa_circ_0049010, hsa_circ_0051307, hsa_circ_0051309, hsa_circ_0040608, hsa_circ_0075505, hsa_circ_0089252, hsa_circ_0089254, hsa_circ_0043926, hsa_circ_0060956, hsa_circ_0030255, hsa_circ_0054031, hsa_circ_0054028, hsa_circ_0024781, hsa_circ_0020846, hsa_circ_0028008, hsa_circ_0016676, hsa_circ_0016674, hsa_circ_0026141, hsa_circ_0058248, hsa_circ_0084117, hsa_circ_0084119, hsa_circ_0071500, hsa_circ_0011024.

The 154 genes screened out in this example had obvious differences in expression levels in the exosomes in the plasma of patients with early acute myocardial infarction and normal population, with abnormal upregulation or downregulation. They could effectively distinguish the patients with early acute myocardial infarction from normal population.

### Example 2 Analysis and Verification of Single Marker Performance

### 1. Analysis of Single Marker Performance

The 154 target sequences (SEQ ID NOs. 1-154) obtained in Example 1 that could distinguish 100 cases of patients with early acute myocardial infarction and 100 cases of normal population were analyzed for individual diagnostic performance respectively, and the patients with early acute myocardial infarction and the normal population were efficiently distinguished according to the abnormal upregulation or downregulation of them. The specific analysis method of the up-regulated genes was shown in Table 1. When the corresponding genes in the sample to be tested had an up-regulated expression fold as shown in Table 1, it could be judged that the patient was at a high risk of being in the hyperacute phase of acute myocardial infarction. The specific analysis method of the down-regulated genes was shown in Table 2. When the corresponding genes in the sample to be tested had an down-regulated expression fold as shown in Table 2, it could be judged that the patient was at a high risk of being in the hyperacute phase of acute myocardial infarction.

TABLE 1
Analysis methods of up-regulated genes

| Marker (SEQ ID NO.) | upregulation fold |
|---|---|
| 1 | 0.67 |
| 2 | 1.54 |
| 3 | 1.03 |
| 4 | 0.51 |
| 5 | 0.52 |
| 6 | 1.20 |
| 7 | 5.25 |
| 8 | 0.59 |
| 9 | 6.09 |
| 10 | 0.60 |
| 11 | 3.86 |
| 12 | 0.87 |
| 13 | 0.91 |
| 14 | 0.81 |
| 15 | 0.35 |
| 16 | 3.66 |
| 17 | 0.35 |
| 18 | 0.37 |
| 19 | 0.64 |
| 20 | 0.65 |
| 21 | 0.53 |
| 22 | 0.58 |
| 23 | 0.89 |
| 24 | 4.44 |
| 25 | 0.53 |
| 26 | 0.58 |
| 27 | 0.69 |
| 28 | 5.82 |
| 38 | 1.51 |
| 99 | 1.54 |
| 100 | 1.56 |
| 101 | 1.58 |

TABLE 2
Analysis methods of down-regulated genes

| Marker (SEQ ID NO.) | Downregulation fold |
|---|---|
| 29 | −0.93 |
| 30 | −0.54 |
| 31 | −0.74 |
| 32 | −0.63 |
| 33 | −0.43 |
| 34 | −0.23 |
| 35 | −0.29 |
| 36 | −1.34 |
| 37 | −0.76 |
| 39 | −1.50 |
| 40 | −1.53 |
| 41 | −1.57 |
| 42 | −1.64 |
| 43 | −1.74 |
| 44 | −1.83 |
| 45 | −1.86 |
| 46 | −1.64 |
| 47 | −1.58 |
| 48 | −1.60 |
| 49 | −1.82 |
| 50 | −1.54 |
| 51 | −1.75 |
| 52 | −2.07 |
| 53 | −1.55 |
| 54 | −1.56 |
| 55 | −1.57 |
| 56 | −1.53 |
| 57 | −1.93 |
| 58 | −1.60 |
| 59 | −1.93 |
| 60 | −1.60 |
| 61 | −1.64 |
| 62 | −1.64 |
| 63 | −1.71 |
| 64 | −1.74 |
| 65 | −2.04 |
| 66 | −1.89 |
| 67 | −1.75 |
| 68 | −1.62 |
| 69 | −1.70 |
| 70 | −2.09 |
| 71 | −1.63 |
| 72 | −1.52 |
| 73 | −1.54 |
| 74 | −1.72 |
| 75 | −1.61 |
| 76 | −1.76 |
| 77 | −1.60 |
| 78 | −1.66 |
| 79 | −1.61 |
| 80 | −1.65 |
| 81 | −1.73 |
| 82 | −1.57 |
| 83 | −1.54 |
| 84 | −1.60 |
| 85 | −1.81 |
| 86 | −1.51 |
| 87 | −1.77 |
| 88 | −1.87 |
| 89 | −1.61 |
| 90 | −1.57 |
| 91 | −1.84 |
| 92 | −1.65 |
| 93 | −1.68 |
| 94 | −1.94 |
| 95 | −1.53 |
| 96 | −1.82 |
| 97 | −1.51 |
| 98 | −1.77 |
| 102 | −1.50 |
| 103 | −2.03 |
| 104 | −1.95 |
| 105 | −1.54 |
| 106 | −1.54 |
| 107 | −1.52 |
| 108 | −1.61 |
| 109 | −1.77 |
| 110 | −1.52 |
| 111 | −1.55 |
| 112 | −1.61 |
| 113 | −1.62 |
| 114 | −1.51 |
| 115 | −1.51 |
| 116 | −1.53 |
| 117 | −1.57 |
| 118 | −1.60 |
| 119 | −1.72 |
| 120 | −1.52 |
| 121 | −1.85 |
| 122 | −1.57 |
| 123 | −1.62 |
| 124 | −2.06 |
| 125 | −1.73 |
| 126 | −1.54 |
| 127 | −1.72 |
| 128 | −1.51 |
| 129 | −1.79 |
| 130 | −1.67 |
| 131 | −1.64 |
| 132 | −1.66 |
| 133 | −1.78 |
| 134 | −1.63 |
| 135 | −1.84 |
| 136 | −1.73 |
| 137 | −1.59 |
| 138 | −1.53 |
| 139 | −1.63 |
| 140 | −1.86 |
| 141 | −1.59 |
| 142 | −1.65 |
| 143 | −1.58 |
| 144 | −1.80 |
| 145 | −1.94 |
| 146 | −1.65 |
| 147 | −1.63 |
| 148 | −1.62 |
| 149 | −1.63 |
| 150 | −1.55 |
| 151 | −2.11 |
| 152 | −1.55 |
| 153 | −1.57 |
| 154 | −1.54 |

According to the methods described in Tables 1 and 2, the target sequences (SEQ ID NOs. 1-153) were analyzed for individual diagnostic performance respectively and their AUC values were calculated. The results were shown in Table 3.

TABLE 3
Comparison of single markers for the diagnosis
of early myocardial infarction

| Marker | |
|---|---|
| (SEQ ID NO.) | AUC |
| 1 | 0.8850 |
| 2 | 0.8050 |
| 3 | 0.7850 |
| 4 | 0.7450 |
| 5 | 0.6900 |
| 6 | 0.7450 |
| 7 | 0.8850 |
| 8 | 0.6500 |
| 9 | 0.9000 |
| 10 | 0.7150 |
| 11 | 0.7550 |
| 12 | 0.6400 |
| 13 | 0.6450 |
| 14 | 0.6200 |
| 15 | 0.7550 |
| 16 | 0.7100 |
| 17 | 0.7250 |
| 18 | 0.6900 |
| 19 | 0.6300 |
| 20 | 0.6900 |
| 21 | 0.8700 |
| 22 | 0.8700 |
| 23 | 0.8250 |
| 24 | 0.7575 |
| 25 | 0.8700 |
| 26 | 0.8700 |
| 27 | 0.7500 |
| 28 | 0.7800 |
| 29 | 0.8400 |
| 30 | 0.7850 |
| 31 | 0.7950 |
| 32 | 0.8150 |
| 33 | 0.8100 |
| 34 | 0.7300 |
| 35 | 0.7250 |
| 36 | 0.8800 |
| 37 | 0.7950 |
| 38 | 0.7200 |
| 39 | 0.8425 |
| 40 | 0.8525 |
| 41 | 0.8350 |
| 42 | 0.8425 |
| 43 | 0.8600 |
| 44 | 0.8750 |
| 45 | 0.8850 |
| 46 | 0.8600 |
| 47 | 0.8150 |
| 48 | 0.8300 |
| 49 | 0.7300 |
| 50 | 0.8600 |
| 51 | 0.7900 |
| 52 | 0.8700 |
| 53 | 0.9125 |
| 54 | 0.8300 |
| 55 | 0.8000 |
| 56 | 0.8500 |
| 57 | 0.8800 |
| 58 | 0.8550 |
| 59 | 0.8150 |
| 60 | 0.8400 |
| 61 | 0.8450 |
| 62 | 0.8975 |
| 63 | 0.8325 |
| 64 | 0.8550 |
| 65 | 0.8600 |
| 66 | 0.8300 |
| 67 | 0.8600 |
| 68 | 0.8300 |
| 69 | 0.8375 |
| 70 | 0.8950 |
| 71 | 0.8500 |
| 72 | 0.8700 |
| 73 | 0.8650 |
| 74 | 0.8700 |
| 75 | 0.8750 |
| 76 | 0.9000 |
| 77 | 0.8550 |
| 78 | 0.8300 |
| 79 | 0.8300 |
| 80 | 0.8400 |
| 81 | 0.8600 |
| 82 | 0.8850 |
| 83 | 0.9650 |
| 84 | 0.8500 |
| 85 | 0.8625 |
| 86 | 0.8250 |
| 87 | 0.8650 |
| 88 | 0.8300 |
| 89 | 0.8300 |
| 90 | 0.8250 |
| 91 | 0.8650 |
| 92 | 0.7900 |
| 93 | 0.8050 |
| 94 | 0.8800 |
| 95 | 0.7950 |
| 96 | 0.8650 |
| 97 | 0.9200 |
| 98 | 0.8550 |
| 99 | 0.8150 |
| 100 | 0.8100 |
| 101 | 0.7625 |
| 102 | 0.7850 |
| 103 | 0.8725 |
| 104 | 0.8700 |
| 105 | 0.9050 |
| 106 | 0.8150 |
| 107 | 0.7750 |
| 108 | 0.8600 |
| 109 | 0.9000 |
| 110 | 0.9050 |
| 111 | 0.8650 |
| 112 | 0.8500 |
| 113 | 0.8150 |
| 114 | 0.8600 |
| 115 | 0.8400 |
| 116 | 0.9000 |
| 117 | 0.8450 |
| 118 | 0.9500 |
| 119 | 0.8900 |
| 120 | 0.8850 |
| 121 | 0.8825 |
| 122 | 0.8550 |
| 123 | 0.8800 |
| 124 | 0.8750 |
| 125 | 0.8300 |
| 126 | 0.8475 |
| 127 | 0.8400 |
| 128 | 0.8350 |
| 129 | 0.7950 |
| 130 | 0.7650 |
| 131 | 0.8350 |
| 132 | 0.8500 |
| 133 | 0.8700 |
| 134 | 0.8400 |
| 135 | 0.8525 |
| 136 | 0.8600 |
| 137 | 0.8450 |
| 138 | 0.8425 |
| 139 | 0.8850 |
| 140 | 0.8350 |
| 141 | 0.8550 |
| 142 | 0.8550 |
| 143 | 0.8250 |
| 144 | 0.8550 |
| 145 | 0.8600 |
| 146 | 0.8600 |
| 147 | 0.8100 |
| 148 | 0.7900 |
| 149 | 0.8700 |
| 150 | 0.8250 |
| 151 | 0.8700 |
| 152 | 0.7925 |
| 153 | 0.8500 |
| 154 | 0.8600 |

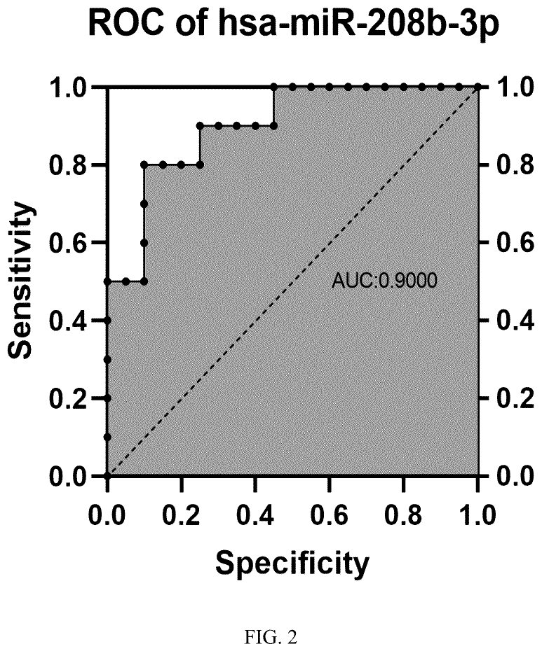

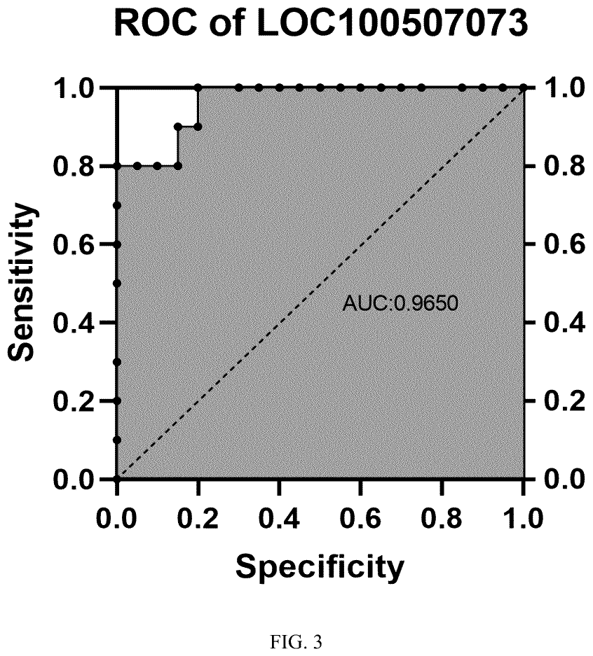

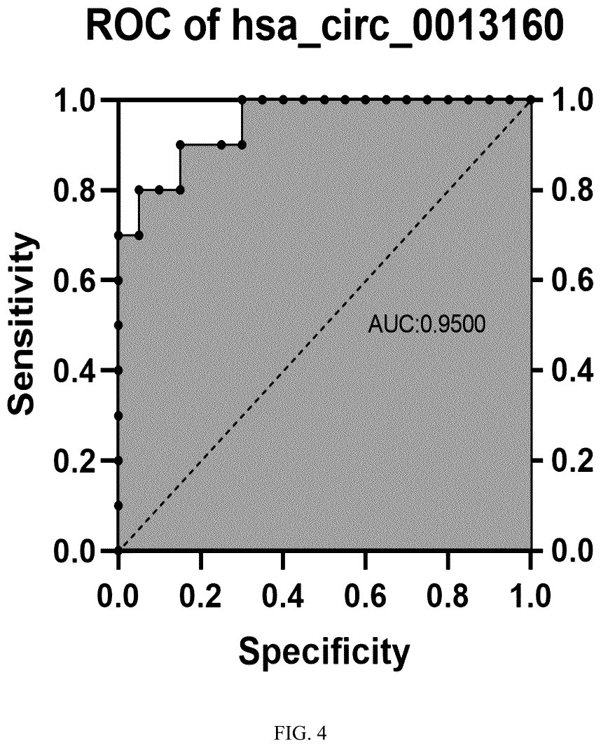

As could be seen from Table 3, the AUC values of 154 target sequences provided in Example 1 for diagnosing acute myocardial infarction in the hyperacute phase were all high, and they all had good diagnostic performance (see FIGS. 2-4, where FIG. 2 was the ROC analysis diagram of SEQ ID NO. 9, FIG. 3 was the ROC analysis diagram of SEQ ID NO. 83, and FIG. 4 was the ROC analysis diagram of SEQ ID NO. 118), which were newly discovered markers that could be used for efficiently screening whether a subject in the hyperacute phase of acute myocardial infarction.

### 2. Clinical Verification of Single Marker Performance

10 target sequences with an AUC higher than 0.9 were selected and used for the diagnosis of 100 cases (as known including 26 cases of positive patients with myocardial infarction and 74 cases of negative normal population) of blood samples. Evaluation and analysis were conducted according to the methods shown in Tables 1 and 2. The primer and probe sequences as employed were shown in Table 4. Diagnostic analysis was conducted on the detection results of fluorescent quantitative PCR. The results of the diagnostic analysis were shown in Table 5.

TABLE 4
Primer probe sequences

| Marker (SEQ ID NO.) | Forward primer | Reverse primer | Probe |
|---|---|---|---|
| 9 | SEQ ID NO. 155 | SEQ ID NO. 156 | SEQ ID NO. 157 |
| 53 | SEQ ID NO. 158 | SEQ ID NO. 159 | SEQ ID NO. 160 |
| 76 | SEQ ID NO. 161 | SEQ ID NO. 162 | SEQ ID NO. 163 |
| 83 | SEQ ID NO. 164 | SEQ ID NO. 165 | SEQ ID NO. 166 |
| 99 | SEQ ID NO. 167 | SEQ ID NO. 168 | SEQ ID NO. 169 |
| 105 | SEQ ID NO. 170 | SEQ ID NO. 171 | SEQ ID NO. 172 |
| 109 | SEQ ID NO. 173 | SEQ ID NO. 174 | SEQ ID NO. 175 |
| 110 | SEQ ID NO. 176 | SEQ ID NO. 177 | SEQ ID NO. 178 |
| 116 | SEQ ID NO. 179 | SEQ ID NO. 180 | SEQ ID NO. 181 |
| 118 | SEQ ID NO. 182 | SEQ ID NO. 183 | SEQ ID NO. 184 |

TABLE 5
Clinical verification of single marker performance

| Marker | Positive | Negative | Accuracy |
|---|---|---|---|
| (SEQ ID NO.) | sample | sample | (%) |
| 9 | 21 | 79 | 80.8 |
| 53 | 23 | 77 | 88.5 |
| 76 | 22 | 78 | 84.6 |
| 83 | 25 | 75 | 96.2 |
| 99 | 24 | 76 | 92.3 |
| 105 | 23 | 77 | 88.5 |
| 109 | 25 | 75 | 96.2 |
| 110 | 22 | 78 | 84.6 |
| 116 | 21 | 79 | 80.8 |
| 118 | 24 | 76 | 92.3 |

The aforementioned single biomarker could be used for diagnosing and distinguishing early myocardial infarction from normal population.

This example also tested and verified the remaining 144 target sequences on 26 cases of positive patients with myocardial infarction and 74 cases of negative normal population, and the accuracy was approximately between 50%-85%. It could be seen that the 154 target sequences provided by the present invention all could be used for diagnosing and distinguishing early myocardial infarction from normal population.

Although the present invention is disclosed above, the present invention is not limited thereto. Various changes and modifications can be made by those of skills in the art without departing from the spirit and scope of the present invention, and thus the claimed scope of the present invention should be based on the scope defined by the claims.

1. A method for predicting whether an individual has early myocardial infarction, comprising:
providing a blood sample of the individual,
testing a marker of the blood sample to obtain the quantity of the marker; and
comparing the obtained quantity with a preset threshold value, and determining whether the individual will have myocardial infarction by the comparison, wherein the marker is any one or more nucleotide sequences selected from miRNA, lncRNA or circRNA, and a sequence of the miRNA is
any one or more selected from the table below:

| Name | Sequence | N | Sequence |
|---|---|---|---|
| hsa-miR-1307-3p_R + 1 | Seq ID NO. 1 | hsa-miR-744-5p_R − 1 | Seq ID NO. 20 |
| hsa-miR-143-3p_R + 1 | Seq ID NO. 2 | hsa-miR-574-5p_R − 2 | Seq ID NO. 21 |
| hsa-miR-27b-3p | Seq ID NO. 3 | hsa-miR-877-5p_R + 2 | Seq ID NO. 22 |
| hsa-miR-152-3p | Seq ID NO. 4 | hsa-miR-1908-5p_R − 1 | Seq ID NO. 23 |
| hsa-miR-24-3p_R − 2 | Seq ID NO. 5 | hsa-miR-490-3p_R + 1 | Seq ID NO. 24 |
| hsa-miR-378a-3p | Seq ID NO. 6 | hsa-miR-574-5p_R − 2 | Seq ID NO. 25 |
| hsa-miR-499a-5p | Seq ID NO. 7 | hsa-miR-877-5p_R + 2 | Seq ID NO. 26 |
| hsa-let-7c-5p | Seq ID NO. 8 | hsa-miR-941 | Seq ID NO. 27 |
| hsa-miR-208b-3p | Seq ID NO. 9 | hsa-miR-9983-3p | Seq ID NO. 28 |
| hsa-miR-584-5p_R − 1 | Seq ID NO. 10 | hsa-miR-375-3p | Seq ID NO. 29 |
| hsa-miR-1-3p | Seq ID NO. 11 | hsa-miR-342-3p | Seq ID NO. 30 |
| hsa-miR-125b-5p | Seq ID NO. 12 | hsa-miR-150-5p | Seq ID NO. 31 |
| hsa-miR-30a-3p_R − 1 | Seq ID NO. 13 | hsa-miR-194-5p_R − 1 | Seq ID NO. 32 |
| hsa-miR-145-3p_L − 2R + 1 | Seq ID NO. 14 | hsa-miR-660-5p | Seq ID NO. 33 |
| hsa-miR-320a-3p | Seq ID NO. 15 | hsa-miR-181a-5p_R − 2 | Seq ID NO. 34 |
| hsa-miR-133a-3p_L − 1R + 1 | Seq ID NO. 16 | hsa-miR-30c-5p_R + 1 | Seq ID NO. 35 |
| hsa-miR-126-5p | Seq ID NO. 17 | hsa-miR-215-5p_R − 1 | Seq ID NO. 36 |
| hsa-miR-423-5p | Seq ID NO. 18 | hsa-miR-200b-3p | Seq ID NO. 37 |
| hsa-miR-378c_R − 5 | Seq ID NO. 19 | | |

2. The method according to claim 1, wherein the lncRNA is any one or more selected from the table below:

| Name | Sequence |
|---|---|
| lnc-DAAM1-2 | Seq ID NO. 38 |
| FTX | Seq ID NO. 39 |
| AC006130.3 | Seq ID NO. 40 |
| CALML3-AS1 | Seq ID NO. 41 |
| CTC-297N7.7 | Seq ID NO. 42 |
| DAB1-AS1 | Seq ID NO. 43 |
| DLEU1 | Seq ID NO. 44 |
| FOXG1-AS1 | Seq ID NO. 45 |
| KCNQ5-IT1 | Seq ID NO. 46 |
| LINC00493 | Seq ID NO. 47 |
| LINC00593 | Seq ID NO. 48 |
| LINC01136 | Seq ID NO. 49 |
| LINC01255 | Seq ID NO. 50 |
| LINC01559 | Seq ID NO. 51 |
| linc-TBX3-5 | Seq ID NO. 52 |
| lnc-AXIN1-1 | Seq ID NO. 53 |
| lnc-B9D1-1 | Seq ID NO. 54 |
| lnc-C12orf42-3 | Seq ID NO. 55 |
| lnc-CDC7-1 | Seq ID NO. 56 |
| lnc-CEPT1-1 | Seq ID NO. 57 |
| lnc-CLDN20-3 | Seq ID NO. 58 |
| lnc-COIL − 2 | Seq ID NO. 59 |
| lnc-DLG4-2 | Seq ID NO. 60 |
| lnc-EIF2AK4-3 | Seq ID NO. 61 |
| lnc-EPHX2-3 | Seq ID NO. 62 |
| lnc-FAM82A2-1 | Seq ID NO. 63 |
| lnc-FOXA2-3 | Seq ID NO. 64 |
| lnc-FOXJ1-2 | Seq ID NO. 65 |
| lnc-KIAA1467-1 | Seq ID NO. 66 |
| lnc-LILRB1-2 | Seq ID NO. 67 |
| lnc-MARCH3-5 | Seq ID NO. 68 |
| lnc-MIS12-4 | Seq ID NO. 69 |
| lnc-OIT3-2 | Seq ID NO. 70 |
| lnc-RAB36-4 | Seq ID NO. 71 |
| lnc-RGL4-2 | Seq ID NO. 72 |
| lnc-SLC17A7-1 | Seq ID NO. 73 |
| lnc-SMC1B-1 | Seq ID NO. 74 |
| lnc-STS-1 | Seq ID NO. 75 |
| lnc-TAAR6-1 | Seq ID NO. 76 |
| lnc-TBCCD1-5 | Seq ID NO. 77 |
| lnc-TCEAL4-1 | Seq ID NO. 78 |
| lnc-TMEM71-3 | Seq ID NO. 79 |
| lnc-TUBA1C-1 | Seq ID NO. 80 |
| lnc-ZDHHC9-1 | Seq ID NO. 81 |
| LOC100505715 | Seq ID NO. 82 |
| LOC100507073 | Seq ID NO. 83 |
| LOC102031319 | Seq ID NO. 84 |
| LOC102724651 | Seq ID NO. 85 |
| LOC153684 | Seq ID NO. 86 |
| LOC338694 | Seq ID NO. 87 |
| RP11-290F20.3 | Seq ID NO. 88 |
| RP11-366H4.1 | Seq ID NO. 89 |
| RP11-383M4.6 | Seq ID NO. 90 |
| RP11-415F23.4 | Seq ID NO. 91 |
| RP5-833A20.1 | Seq ID NO. 92 |
| RPL23AP32 | Seq ID NO. 93 |
| TMEM147-AS1 | Seq ID NO. 94 |
| XLOC_l2_001089 | Seq ID NO. 95 |
| XLOC_l2_006640 | Seq ID NO. 96 |
| XLOC_l2_007731 | Seq ID NO. 97 |
| XLOC_l2_009804 | Seq ID NO. 98 |

3. The method according to claim 2, wherein the circRNA is any one or more selected from the table below:

| Name | Sequence |
|---|---|
| hsa_circ_0044880 | Seq ID NO. 99 |
| hsa_circ_0032837 | Seq ID NO. 100 |
| hsa_circ_0063420 | Seq ID NO. 101 |
| hsa_circ_0046151 | Seq ID NO. 102 |
| hsa_circ_0037779 | Seq ID NO. 103 |
| hsa_circ_0037777 | Seq ID NO. 104 |
| hsa_circ_0058356 | Seq ID NO. 105 |
| hsa_circ_0074657 | Seq ID NO. 106 |
| hsa_circ_0092053 | Seq ID NO. 107 |
| hsa_circ_0049892 | Seq ID NO. 108 |
| hsa_circ_0057694 | Seq ID NO. 109 |
| hsa_circ_0071517 | Seq ID NO. 110 |
| hsa_circ_0072207 | Seq ID NO. 111 |
| hsa_circ_0045961 | Seq ID NO. 112 |
| hsa_circ_0089955 | Seq ID NO. 113 |
| hsa_circ_0024057 | Seq ID NO. 114 |
| hsa_circ_0002512 | Seq ID NO. 115 |
| hsa_circ_0038850 | Seq ID NO. 116 |
| hsa_circ_0012424 | Seq ID NO. 117 |
| hsa_circ_0013160 | Seq ID NO. 118 |
| hsa_circ_0020080 | Seq ID NO. 119 |
| hsa_circ_0020079 | Seq ID NO. 120 |
| hsa_circ_0041015 | Seq ID NO. 121 |
| hsa_circ_0041019 | Seq ID NO. 122 |
| hsa_circ_0055058 | Seq ID NO. 123 |
| hsa_circ_0027427 | Seq ID NO. 124 |
| hsa_circ_0027430 | Seq ID NO. 125 |
| hsa_circ_0072550 | Seq ID NO. 126 |
| hsa_circ_0008774 | Seq ID NO. 127 |
| hsa_circ_0064438 | Seq ID NO. 128 |
| hsa_circ_0007281 | Seq ID NO. 129 |
| hsa_circ_0069099 | Seq ID NO. 130 |
| hsa_circ_0066990 | Seq ID NO. 131 |
| hsa_circ_0049010 | Seq ID NO. 132 |
| hsa_circ_0051307 | Seq ID NO. 133 |
| hsa_circ_0051309 | Seq ID NO. 134 |
| hsa_circ_0040608 | Seq ID NO. 135 |
| hsa_circ_0075505 | Seq ID NO. 136 |
| hsa_circ_0089252 | Seq ID NO. 137 |
| hsa_circ_0089254 | Seq ID NO. 138 |
| hsa_circ_0043926 | Seq ID NO. 139 |
| hsa_circ_0060956 | Seq ID NO. 140 |
| hsa_circ_0030255 | Seq ID NO. 141 |
| hsa_circ_0054031 | Seq ID NO. 142 |
| hsa_circ_0054028 | Seq ID NO. 143 |
| hsa_circ_0024781 | Seq ID NO. 144 |
| hsa_circ_0020846 | Seq ID NO. 145 |
| hsa_circ_0028008 | Seq ID NO. 146 |
| hsa_circ_0016676 | Seq ID NO. 147 |
| hsa_circ_0016674 | Seq ID NO. 148 |
| hsa_circ_0026141 | Seq ID NO. 149 |
| hsa_circ_0058248 | Seq ID NO. 150 |
| hsa_circ_0084117 | Seq ID NO. 151 |
| hsa_circ_0084119 | Seq ID NO. 152 |
| hsa_circ_0071500 | Seq ID NO. 153 |
| hsa_circ_0011024 | Seq ID NO. 154 |

4. The method according to claim 1, wherein the myocardial infarction comprises myocardial infarction in a hyperacute phase, and the hyperacute phase refers to a time window range after the patient suffers from acute myocardial infarction.

5. The method according to claim 4, wherein the early myocardial infarction comprises a hyperacute phase, and the hyperacute phase means that the patient will progress to acute myocardial infarction within 6 hours or less.

6. The method according to claim 1, wherein the threshold value is the content of the marker in healthy people, and the fold of the marker being up-regulated or down-regulated is used for predicting whether a patient will suffer from myocardial infarction.

7. The method according to claim 6, wherein when the nucleotide sequence shown in the table below occurs in the blood sample of an individual with the upregulation fold shown in the table below, it can be judged that the individual has a high risk of hyperacute phase of acute myocardial infarction,

| Marker (SEQ | upregulation |
|---|---|
| ID NO.) | fold |
| 1 | 0.67 |
| 2 | 1.54 |
| 3 | 1.03 |
| 4 | 0.51 |
| 5 | 0.52 |
| 6 | 1.20 |
| 7 | 5.25 |
| 8 | 0.59 |
| 9 | 6.09 |
| 10 | 0.60 |
| 11 | 3.86 |
| 12 | 0.87 |
| 13 | 0.91 |
| 14 | 0.81 |
| 15 | 0.35 |
| 16 | 3.66 |
| 17 | 0.35 |
| 18 | 0.37 |
| 19 | 0.64 |
| 20 | 0.65 |
| 21 | 0.53 |
| 22 | 0.58 |
| 23 | 0.89 |
| 24 | 4.44 |
| 25 | 0.53 |
| 26 | 0.58 |
| 27 | 0.69 |
| 28 | 5.82 |
| 38 | 1.51 |
| 99 | 1.54 |
| 100 | 1.56 |
| 101 | 1.58 |

8. The method according to claim 6, wherein when the nucleotide sequence shown in the table below occurs in the blood sample of an individual with the downregulation fold shown in the table below, it can be judged that the individual has a high risk of hyperacute phase of acute myocardial infarction,

| Marker (SEQ ID NO.) | Downregulation fold |
|---|---|
| 29 | −0.93 |
| 30 | −0.54 |
| 31 | −0.74 |
| 32 | −0.63 |
| 33 | −0.43 |
| 34 | −0.23 |
| 35 | −0.29 |
| 36 | −1.34 |
| 37 | −0.76 |
| 39 | −1.50 |
| 40 | −1.53 |
| 41 | −1.57 |
| 42 | −1.64 |
| 43 | −1.74 |
| 44 | −1.83 |
| 45 | −1.86 |
| 46 | −1.64 |
| 47 | −1.58 |
| 48 | −1.60 |
| 49 | −1.82 |
| 50 | −1.54 |
| 51 | −1.75 |
| 52 | −2.07 |
| 53 | −1.55 |
| 54 | −1.56 |
| 55 | −1.57 |
| 56 | −1.53 |
| 57 | −1.93 |
| 58 | −1.60 |
| 59 | −1.93 |
| 60 | −1.60 |
| 61 | −1.64 |
| 62 | −1.64 |
| 63 | −1.71 |
| 64 | −1.74 |
| 65 | −2.04 |
| 66 | −1.89 |
| 67 | −1.75 |
| 68 | −1.62 |
| 69 | −1.70 |
| 70 | −2.09 |
| 71 | −1.63 |
| 72 | −1.52 |
| 73 | −1.54 |
| 74 | −1.72 |
| 75 | −1.61 |
| 76 | −1.76 |
| 77 | −1.60 |
| 78 | −1.66 |
| 79 | −1.61 |
| 80 | −1.65 |
| 81 | −1.73 |
| 82 | −1.57 |
| 83 | −1.54 |
| 84 | −1.60 |
| 85 | −1.81 |
| 86 | −1.51 |
| 87 | −1.77 |
| 88 | −1.87 |
| 89 | −1.61 |
| 90 | −1.57 |
| 91 | −1.84 |
| 92 | −1.65 |
| 93 | −1.68 |
| 94 | −1.94 |
| 95 | −1.53 |
| 96 | −1.82 |
| 97 | −1.51 |
| 98 | −1.77 |
| 102 | −1.50 |
| 103 | −2.03 |
| 104 | −1.95 |
| 105 | −1.54 |
| 106 | −1.54 |
| 107 | −1.52 |
| 108 | −1.61 |
| 109 | −1.77 |
| 110 | −1.52 |
| 111 | −1.55 |
| 112 | −1.61 |
| 113 | −1.62 |
| 114 | −1.51 |
| 115 | −1.51 |
| 116 | −1.53 |
| 117 | −1.57 |
| 118 | −1.60 |
| 119 | −1.72 |
| 120 | −1.52 |
| 121 | −1.85 |
| 122 | −1.57 |
| 123 | −1.62 |
| 124 | −2.06 |
| 125 | −1.73 |
| 126 | −1.54 |
| 127 | −1.72 |
| 128 | −1.51 |
| 129 | −1.79 |
| 130 | −1.67 |
| 131 | −1.64 |
| 132 | −1.66 |
| 133 | −1.78 |
| 134 | −1.63 |
| 135 | −1.84 |
| 136 | −1.73 |
| 137 | −1.59 |
| 138 | −1.53 |
| 139 | −1.63 |
| 140 | −1.86 |
| 141 | −1.59 |
| 142 | −1.65 |
| 143 | −1.58 |
| 144 | −1.80 |
| 145 | −1.94 |
| 146 | −1.65 |
| 147 | −1.63 |
| 148 | −1.62 |
| 149 | −1.63 |
| 150 | −1.55 |
| 151 | −2.11 |
| 152 | −1.55 |
| 153 | −1.57 |
| 154 | −1.54 |

9. The method according to claim 1, wherein a nucleic acid sequence of the RNA is one of sequences as shown in SEQ ID NO. 9, SEQ ID NO. 83, and SEQ ID NO. 118.

10. The method according to claim 9, wherein a sequence as shown in SEQ ID NO. 155 is used as a forward primer, a sequence as shown in SEQ ID NO. 156 is used as a reverse primer, and a sequence as shown in SEQ ID NO. 157 is used as a probe to test the content of the sequence as shown in SEQ ID NO. 9 in the blood sample.

11. The method according to claim 9, wherein a sequence as shown in SEQ ID NO. 164 is used as a forward primer, a sequence as shown in SEQ ID NO. 165 is used as a reverse primer, and a sequence as shown in SEQ ID NO. 166 is used as a probe to test the content of the sequence as shown in SEQ ID NO. 83 in the blood sample.

12. The method according to claim 9, wherein a sequence as shown in SEQ ID NO. 182 is used as a forward primer, a sequence as shown in SEQ ID NO. 183 is used as a reverse primer, and a sequence as shown in SEQ ID NO. 184 is used as a probe to test the content of the sequence as shown in SEQ ID NO. 118 in the blood sample.

13. The method according to claim 1, wherein the blood sample comprises an exosome, and the quantity of a marker of the exosome is tested.
